(19) United States
(12) Patent Application Publication
(10) Pub. No.: US 2012/0237536 A1
(43) Pub. Date: Sep. 20, 2012

(54) COMBINATION VACCINES AGAINST RESPIRATORY TRACT DISEASES

(75) Inventors: Rino Rappuoli, CasteInuovo Berardenga (IT); Ralf Leo Clemens, Rio De Janeiro (BR)

(73) Assignee: NOVARTIS, Basel (CH)

(21) Appl. No.: 13/395,434

(22) PCT Filed: Sep. 10, 2010
(86) PCT No.: PCT/IB2010/002401

§ 371 Date: May 31, 2012

Related U.S. Application Data
(60) Provisional application No. 61/241,264, filed on Sep. 10, 2009.
(60) Provisional application No. 61/241,485, filed on Sep. 11, 2009.

(51) Int. Cl. A61K 39/295; A61P 31/04; A61P 31/14; A61P 31/16

(52) U.S. Cl. 424/190.1; 424/201.1

(57) ABSTRACT

Influenza, pneumococcus and/or RSV vaccines are administered as a combination vaccine while retaining immunogenic efficacy. This combination simplifies immunisation against these two lower respiratory tract infections. The pneumococcal vaccine ideally includes at least one pneumococcal polypeptide.

This application claims the benefit of U.S. provisional applications 61/241,264 filed Sep. 10, 2009 and 61/241,485 filed Sep. 11, 2009, the complete contents of both of which are hereby incorporated herein by reference for all purposes.

### TECHNICAL FIELD

This invention is in the field of immunisation against lower and/or upper respiratory tract diseases.

### BACKGROUND ART

It is known to co-administer different respiratory vaccines to a subject at the same time e.g. to administer a pneumococcal vaccine at the same time as an influenza vaccine (e.g. refs 1 to 4). Combination vaccines, in which two or more vaccines are administered as a mixture, are also known e.g. reference 5 combined pneumococcal saccharides (conjugated or unconjugated) with a respiratory syncytial virus (RSV) antigen, and also speculated that a number of other antigens such as an influenza virus antigen might be added. Reference 6 discloses combinations of the fusion (F), attachment (G) and matrix (M) proteins of RSV with an influenza vaccine. Reference 7 discloses a combination vaccine against influenza A virus and RSV based on administering plasmids.

It is an object of the invention to simplify immunisation against lower and/or upper respiratory tract diseases.

### DISCLOSURE OF THE INVENTION

Whereas references 1 to 4 (and various other documents) have co-administered separate influenza and pneumococcus vaccines, the inventors have found that such vaccines can be administered as a combination vaccine while retaining immunogenic efficacy. Whereas reference 5 included a RSV antigen, the inventors provide a combination of influenza and pneumococcus vaccines without necessarily including a RSV component. Moreover, in contrast to reference 5, the inventors prefer to include pneumococcal protein antigens rather than relying solely on pneumococcal saccharide antigens. Also, the inclusion of a pneumococcal immunogen (including protein and/or saccharide components) can improve the vaccines of references 6 and 7. These findings mean that immunisation against these different lower respiratory tract infections can be simplified and improved.

Combining influenza and pneumococcus vaccines is not trivial. Whereas current pneumococcal vaccines (e.g. the PREVNAR™ and SYNFLORIX™ products) have fixed compositions and are administered at any time of the year, the composition of influenza vaccines varies from season-to-season and the vaccine is administered at the start of winter. Thus the two vaccines are a priori incompatible, but the inventors show that the combination is feasible.

Thus the invention provides an immunogenic composition comprising an influenza virus immunogen and a pneumococcal immunogen. These compositions are suitable for immunisation against both influenza virus and pneumococcus. The pneumococcal immunogen will typically comprise at least one pneumococcal polypeptide. The composition may include a RSV immunogen, but in some embodiments the composition does not include a RSV immunogen.

The invention also provides an immunogenic composition comprising (i) a pneumococcal immunogen comprising at least one pneumococcal polypeptide and (ii) an influenza virus immunogen and/or a RSV immunogen. In some embodiments the composition does not include a RSV immunogen, and in some embodiments the composition does not include an influenza virus immunogen, but in some embodiments it includes both a RSV immunogen and an influenza virus immunogen.

The invention also provides a process for preparing an immunogenic composition, comprising a step of admixing an influenza virus immunogen and a pneumococcal immunogen. The pneumococcal immunogen will typically comprise at least one pneumococcal polypeptide. The immunogenic composition can be a composition which does not include a RSV immunogen.

The invention also provides a process for preparing an immunogenic composition, comprising a step of admixing a pneumococcal immunogen comprising at least one pneumococcal polypeptide with one or both of a RSV immunogen and an influenza virus immunogen. Where the composition includes all three of a pneumococcal immunogen, a RSV immunogen and an influenza virus immunogen, these components may be mixed in any order.

These processes of the invention may provide a composition with a unit dose volume of 0.5 ml.

Compositions of the invention can also be made suitable for additionally immunising against group B streptococcus (Streptococcus agalactiae; GBS). Thus, in some embodiments, the composition also includes a GBS immunogen e.g. a combination of influenza, pneumococcus and GBS immunogens (with or without a RSV immunogen). A process of the invention may include a step of admixing a GBS immunogen with (i) the influenza virus immunogen, (ii) the pneumococcal immunogen, (iii) the RSV immunogen, and/or (iv) a mixture of any 1, 2 or 3 of the influenza virus immunogen, the pneumococcal immunogen and the RSV immunogen.

### The Influenza Virus Immunogen

The influenza virus immunogen can take various forms. Influenza vaccines are generally based either on live virus or on inactivated virus, and the invention preferably uses an inactivated virus as the influenza immunogen. An inactivated virus immunogen may be based on whole virions, ‘split’ virions, or on purified surface antigens (including hemagglutinin and, usually, also including neuraminidase). Another type of influenza virus immunogen which may be used with the invention is a virosome. The invention may also use recombinant hemagglutinin and/or neuraminidase glycoprotein(s) as the influenza virus immunogen. A further useful type of influenza virus immunogen is the M2 matrix protein. Live attenuated vaccines can be used with the invention, but would typically be used only in combination with a live attenuated RSV vaccine.

For preparing inactivated virus immunogen, chemical means for inactivating a virus include treatment with an effective amount of one or more of the following agents: detergents, formaldehyde, β-propiolactone, methylene blue, psoralen, carboxyfullerene (C60), binary ethylamine, acetyl ethyleneimine, or combinations thereof. Non-chemical methods of viral inactivation are known in the art, such as for example UV light or gamma irradiation.

Virions can be harvested from virus-containing fluids by various methods. For example, a purification process may involve zonal centrifugation using a linear sucrose gradient solution that includes detergent to disrupt the virions. Antigens may then be purified, after optional dilution, by diafiltration.

Split virions are obtained by treating purified virions with detergents to produce subvirion preparations, including the ‘Tween-ether’ splitting process. Methods of splitting influenza viruses are well known in the art e.g. see refs. 8-13, etc. Splitting of the virus is typically carried out by disrupting or fragmenting whole virus, whether infectious or non-infectious with a disrupting concentration of a splitting agent. The disruption results in a full or partial solubilisation of the virus proteins, altering the integrity of the virus. Preferred splitting agents are non-ionic and ionic (e.g. cationic) surfactants e.g. ethyl ether, deoxycholate, tri-N-butyl phosphate, Tergitol NP9, alkylglycosides, alkylthioglycosides, acyl sugars, sulphobetaines, betains, polyoxyethylenealkylethers, N,N-dialkyl-Glucamides, Hecameg, alkylphenoxy-polyethoxyethanols, quaternary ammonium compounds, sarcosyl, CTABs (cetyl trimethyl ammonium bromides e.g. Cetavlon), tri-N-butyl phosphate, myristyltrimethylammonium salts, lipofectin, lipofectamine, and DOTMA, the octyl- or nonylphenoxy polyoxyethanols (e.g. the Triton surfactants, such as Triton X-100 or Triton N101), polyoxyethylene sorbitan esters (the Tween surfactants e.g. polysorbate 80), polyoxyethylene ethers, polyoxyethlene esters, etc. One useful splitting procedure uses the consecutive effects of sodium deoxycholate and formaldehyde, and splitting can take place during initial virion purification (e.g. in a sucrose density gradient solution). Thus a splitting process can involve clarification of the virion-containing material (to remove non-virion material), concentration of the harvested virions (e.g. using an adsorption method, such as CaHPO₄ adsorption), separation of whole virions from non-virion material, splitting of virions using a splitting agent in a density gradient centrifugation step (e.g. using a sucrose gradient that contains a splitting agent such as sodium deoxycholate), and then filtration (e.g. ultrafiltration) to remove undesired materials. Split virions can usefully be resuspended in sodium phosphate-buffered isotonic sodium chloride solution. The BEGRIVAC™, FLUARIX™, FLUZONE™ and FLUSHIELD™ products are split vaccines.

Purified surface antigens comprise the influenza surface antigens haemagglutinin and, typically, also neuraminidase. They are obtained by purification of these glycoproteins from influenza virions. Processes for preparing these proteins in purified form are well known in the art. The FLUVIRIN™, AGRIPPAL™ and INFLUVAC™ products are subunit vaccines.

Another useful influenza antigen is the virosome [14] (i.e. nucleic acid free viral-like liposomal particles) as in the INFLEXAL V™ and INVAVAC™ products. Virosomes can be prepared by solubilization of influenza virus with a detergent followed by removal of the nucleocapsid and reconstitution of the membrane containing the viral glycoproteins. An alternative method for preparing virosomes involves adding viral membrane glycoproteins to excess amounts of phospholipids, to give liposomes with viral proteins in their membrane.

As an alternative to making influenza vaccines from material derived from influenza virions, it is also known to express proteins in heterologous recombinant hosts. For example, HA can be expressed in an insect cell line using a baculovirus vector [15,16], as can neuraminidase [17]. Purified recombinant hemagglutinin and/or neuraminidase glycoprotein(s) can be used as immunogens with the invention. These recombinant antigens may be full-length or may comprise epitopes from full-length proteins e.g. including a HA ectodomain.

A further useful type of influenza virus immunogen is the M2 matrix protein. It is known to use the M2 ectodomain (M2e; 20-25 amino acids in length) for immunising against influenza. M2e can be fused to a protein such as the hepatitis B core antigen (HBc) to provide immunogenic particles which present M2 antigen on their surface. Fusion to proteins such as GCN4 can also provide oligomeric M2e. Such recombinant M2e fusion proteins can be used with the invention.

Where the influenza virus immunogen comprises hemagglutinin, more than one hemagglutinin may be included. The hemagglutinin of circulating influenza viruses changes over time and so vaccine immunogens are kept up to date every season. Thus an influenza virus immunogen may be multivalent e.g. including at least one influenza A virus hemagglutinin and at least one influenza B virus hemagglutinin, including at least two different influenza A virus hemagglutinins, including at least two different influenza B virus hemagglutinins, etc. For example, a composition may include hemagglutinin from two influenza A strains (H1N1 and H3N2) and one influenza B strain. Where two influenza A virus hemagglutinins are included from different subtypes (e.g. H1 and H3), if neuraminidase is included then ideally two different neuraminidase subtypes are also included (e.g. N1 and N2). In some embodiments, though, different hemagglutinin subtypes but identical neuraminidase subtypes are included (e.g. a combination of H1N1 and H5N1).

In other embodiments, a hemagglutinin-containing influenza virus immunogen may be monovalent i.e. including hemagglutinin from only one influenza virus strain. Such monovalent immunogens will typically be from an influenza A virus e.g. from any one of subtypes H1, H2, H3, H4, H5, H6, H7, H8, H9, H10, H11, H12, H13, H14, H15 or H16. Monovalent immunogens are particularly useful with pandemic strains, including strains to which the vaccine recipient and the general human population are immunologically naïve, such as H2, H5, H7 or H9 subtype influenza A virus strains.

More generally, the influenza virus immunogen may include hemagglutinin from: (i) one or more (e.g. 1, 2, 3, 4, 5 or more) strains of influenza A virus of hemagglutinin subtype H1, H2, H3, H4, H5, H6, H7, H8, H9, H10, H11, H12, H13, H14, H15 and/or H16; and/or (ii) one or more (e.g. 1, 2, 3, 4, 5 or more) strains of influenza B virus. Where more than one influenza B virus hemagglutinin is included, it is useful to include hemagglutinin from each of a B/Victoria/2/87-like strain and a B/Yamagata/16/88-like strain. These two types of strain are usually distinguished antigenically, but differences in amino acid sequences have also been described for distinguishing the two lineages e.g. B/Yamagata/16/88-like strains often (but not always) have HA proteins with deletions at amino acid residue 164, numbered relative to the ‘Lee40’ HA sequence [18].

Specific embodiments of suitable influenza virus immunogens for use with the invention include, but are not limited to, immunogens including hemagglutinin from: (i) a trivalent combination of a H1N1 influenza A virus, a H3N2 influenza A virus, and an influenza B virus; (ii) a monovalent H5N1 influenza A virus; (iii) a tetravalent combination of a H1N1 influenza A virus, a H3N2 influenza A virus, a B/Victoria/2/87-like influenza B virus and a B/Yamagata/16/88-like influenza B virus; (iv) a tetravalent combination of a H1N1 influenza A virus, a H3N2 influenza A virus, a H5N1 influenza A virus, and an influenza B virus.

Hemagglutinin (HA) is the main influenza virus immunogen in current inactivated influenza vaccines, all of which contain HA, and vaccine doses are standardised by reference to the HA levels, typically measured by SRID. Existing vaccines typically contain about 15 μg of HA per strain, although lower doses can be used e.g. for children, or in pandemic situations, or when using an adjuvant. Fractional doses such as ½ (i.e. 7.5 μg HA per strain), ¼ and ⅛ have been used [19,20], as have higher doses (e.g. 3× or 9× doses [21,22]). Thus vaccines may include between 0.1 and 150 μg of HA per influenza strain, preferably between 0.1 and 50 μg e.g. 0.1-20 μg, 0.1-15 μg, 0.1-10 μg, 0.1-7.5 μg, 0.5-5 μg, etc. Particular doses include e.g. about 45, about 30, about 15, about 10, about 7.5, about 5, about 3.8, about 1.9, about 1.5, etc. per strain. It is preferred to use substantially the same mass of HA for each strain included in the vaccine e.g. such that the HA mass for each strain is within 10% of the mean HA mass per strain, and preferably within 5% of the mean.

The hemagglutinin in an influenza virus immunogen may be a natural HA as found in a wild-type virus, or a modified HA. For instance, it is known to modify HA to remove determinants (e.g. hyper-basic regions around the HA1/HA2 cleavage site) that cause a virus to be highly pathogenic in avian species.

A hemagglutinin in the influenza virus immunogen ideally has a binding preference for oligosaccharides with a Sia(α2,6)Gal terminal disaccharide compared to oligosaccharides with a Sia(α2,3)Gal terminal disaccharide. Human influenza viruses bind to receptor oligosaccharides having a Sia(α2,6)Gal terminal disaccharide (sialic acid linked α-2,6 to galactose), but eggs and Vero cells have receptor oligosaccharides with a Sia(α2,3)Gal terminal disaccharide. Growth of human influenza viruses in cells such as MDCK provides selection pressure on hemagglutinin to maintain the native Sia(α2,6)Gal binding, unlike egg passaging. To determine if a virus has a binding preference for oligosaccharides with a Sia(α2,6)Gal terminal disaccharide compared to oligosaccharides with a Sia(α2,3)Gal terminal disaccharide, various assays can be used. For instance, reference 23 describes a solid-phase enzyme-linked assay for influenza virus receptor-binding activity which gives sensitive and quantitative measurements of affinity constants. Reference 24 used a solid-phase assay in which binding of viruses to two different sialylglycoproteins was assessed (ovomucoid, with Sia(α2,3)Gal determinants; and pig α₂-macroglobulin, which Sia(α2,6)Gal determinants), and also describes an assay in which the binding of virus was assessed against two receptor analogs: free sialic acid (Neu5Ac) and 3′-sialyllactose (Neu5Acα2-3Galβ1-4Glc). Reference 25 reports an assay using a glycan array which was able to clearly differentiate receptor preferences for α2,3 or α2,6 linkages. Reference 26 reports an assay based on agglutination of human erythrocytes enzymatically modified to contain either Sia(α2,6)Gal or Sia(α2,3)Gal. Depending on the type of assay, it may be performed directly with the virus itself, or can be performed indirectly with hemagglutinin purified from the virus.

In some embodiments a hemagglutinin (and other influenza virus glycoprotein(s) present in the influenza virus immunogen) has a different glycosylation pattern from egg-derived viruses. Thus the glycoproteins will include glycoforms that are not seen in chicken eggs.

Where an influenza virus immunogen is prepared from influenza virions, these will have been produced in a suitable substrate. Substrates currently in use for growing influenza viruses include eggs and cell culture. The current standard method for influenza virus growth uses specific pathogen-free (SPF) embryonated hen eggs, with virions being purified from the egg contents (allantoic fluid). As an alternative, however, viruses have been grown in animal cell culture and, for reasons of speed and patient allergies, this growth method is preferred.

For such cell culture methods, virus will usually be grown in a cell line of mammalian origin. Suitable mammalian cells of origin include, but are not limited to, hamster, cattle, primate (including humans and monkeys) and dog cells, although the use of primate cells is not preferred. Various cell types may be used, such as kidney cells, fibroblasts, retinal cells, lung cells, etc. Examples of suitable hamster cells are the cell lines having the names BHK21 or HKCC. Suitable monkey cells are e.g. African green monkey cells, such as kidney cells as in the Vero cell line [27-29]. Suitable dog cells are e.g. kidney cells, as in the CLDK and MDCK cell lines.

Thus suitable cell lines include, but are not limited to: MDCK; CHO; CLDK; HKCC; 293T; BHK; Vero; MRC-5; PER.C6 [30]; FRhL2; WI-38; etc. Suitable cell lines are widely available e.g. from the American Type Cell Culture (ATCC) collection [31], from the Coriell Cell Repositories [32], or from the European Collection of Cell Cultures (ECACC). For example, the ATCC supplies various different Vero cells under catalog numbers CCL-81, CCL-81.2, CRL-1586 and CRL-1587, and it supplies MDCK cells under catalog number CCL-34. PER.C6 is available from the ECACC under deposit number 96022940.

The most preferred cell lines are those with mammalian-type glycosylation. As a less-preferred alternative to mammalian cell lines, virus can be grown on avian cell lines [e.g. refs. 33-35], including cell lines derived from ducks (e.g. duck retina) or hens. Examples of avian cell lines include avian embryonic stem cells [33,36] and duck retina cells [34]. Suitable avian embryonic stem cells, include the EBx cell line derived from chicken embryonic stem cells, EB45, EB14, and EB14-074 [37]. Chicken embryo fibroblasts (CEF) may also be used. Rather than using avian cells, however, the use of mammalian cells means that vaccines can be free from avian DNA and egg proteins (such as ovalbumin and ovomucoid), thereby reducing allergenicity.

The most preferred cell lines for growing influenza viruses are MDCK cell lines [38-41], derived from Madin Darby canine kidney. The original MDCK cell line is available from the ATCC as CCL-34, but derivatives of this cell line may also be used. For instance, reference 38 discloses a MDCK cell line that was adapted for growth in suspension culture (‘MDCK 33016’, deposited as DSM ACC 2219). Similarly, reference 42 discloses a MDCK-derived cell line that grows in suspension in serum-free culture (‘B-702’, deposited as FERM BP-7449). Reference 43 discloses non-tumorigenic MDCK cells, including ‘MDCK-S’ (ATCC PTA-6500), ‘MDCK-SF101’ (ATCC PTA-6501), ‘MDCK-SF102’ (ATCC PTA-6502) and ‘MDCK-SF103’ (PTA-6503). Reference 44 discloses MDCK cell lines with high susceptibility to infection, including ‘MDCK.5F1’ cells (ATCC CRL-12042). Any of these MDCK cell lines can be used.

Virus may be grown on cells in adherent culture or in suspension. Microcarrier cultures can also be used. In some embodiments, the cells may thus be adapted for growth in suspension.

Cell lines are preferably grown in serum-free culture media and/or protein free media. A medium is referred to as a serum-free medium in the context of the present invention in which there are no additives from serum of human or animal origin. The cells growing in such cultures naturally contain proteins themselves, but a protein-free medium is understood to mean one in which multiplication of the cells occurs with exclusion of proteins, growth factors, other protein additives and non-serum proteins, but can optionally include proteins such as trypsin or other proteases that may be necessary for viral growth.

Cell lines supporting influenza virus replication are preferably grown below 37° C. [45] (e.g. 30-36° C., or at about 30° C., 31° C., 32° C., 33° C., 34° C., 35° C., 36° C.) during viral replication.

Methods for propagating influenza virus in cultured cells generally includes the steps of inoculating a culture of cells with an inoculum of the strain to be grown, cultivating the infected cells for a desired time period for virus propagation, such as for example as determined by virus titer or antigen expression (e.g. between 24 and 168 hours after inoculation) and collecting the propagated virus. The cultured cells are inoculated with a virus (measured by PFU or TCID₅₀) to cell ratio of 1:500 to 1:1, preferably 1:100 to 1:5, more preferably 1:50 to 1:10. The virus is added to a suspension of the cells or is applied to a monolayer of the cells, and the virus is absorbed on the cells for at least 60 minutes but usually less than 300 minutes, preferably between 90 and 240 minutes at 25° C. to 40° C., preferably 28° C. to 37° C. The infected cell culture (e.g. monolayers) may be removed either by freeze-thawing or by enzymatic action to increase the viral content of the harvested culture supernatants. The harvested fluids are then either inactivated or stored frozen. Cultured cells may be infected at a multiplicity of infection (“m.o.i.”) of about 0.0001 to 10, preferably 0.002 to 5, more preferably to 0.001 to 2. Still more preferably, the cells are infected at a m.o.i of about 0.01. Infected cells may be harvested 30 to 60 hours post infection. Preferably, the cells are harvested 34 to 48 hours post infection. Still more preferably, the cells are harvested 38 to 40 hours post infection. Proteases (typically trypsin) are generally added during cell culture to allow viral release, and the proteases can be added at any suitable stage during the culture e.g. before inoculation, at the same time as inoculation, or after inoculation [45].

In preferred embodiments, particularly with MDCK cells, a cell line is not passaged from the master working cell bank beyond 40 population-doubling levels.

The viral inoculum and the viral culture are preferably free from (i.e. will have been tested for and given a negative result for contamination by) herpes simplex virus, respiratory syncytial virus, parainfluenza virus 3, SARS coronavirus, adenovirus, rhinovirus, reoviruses, polyomaviruses, birnaviruses, circoviruses, and/or parvoviruses [46]. Absence of herpes simplex viruses is particularly preferred.

Where virus has been grown on a cell line it is standard practice to minimize the amount of residual cell line DNA in the final vaccine, in order to minimize any oncogenic activity of the DNA. Thus a composition prepared from culture-grown influenza viruses preferably contains less than 10 ng (preferably less than 1 ng, and more preferably less than 100 pg) of residual host cell DNA per dose, although trace amounts of host cell DNA may be present. Vaccines containing <10 ng (e.g. <1 ng, <100 pg) host cell DNA per 15 μg of haemagglutinin are preferred, as are vaccines containing <10 ng (e.g. <1 ng, <100 pg) host cell DNA per 0.25 ml volume. Vaccines containing <10 ng (e.g. <1 ng, <100 pg) host cell DNA per 50 μg of haemagglutinin are more preferred, as are vaccines containing <10 ng (e.g. <1 ng, <100 pg) host cell DNA per 0.5 ml volume.

It is preferred that the average length of any residual host cell DNA is less than 500 bp e.g. less than 400 bp, less than 300 bp, less than 200 bp, less than 100 bp, etc.

Contaminating DNA can be removed during vaccine preparation using standard purification procedures e.g. chromatography, etc. Removal of residual host cell DNA can be enhanced by nuclease treatment e.g. by using a DNase. A convenient method for reducing host cell DNA contamination is disclosed in references 47 & 48, involving a two-step treatment, first using a DNase (e.g. Benzonase), which may be used during viral growth, and then a cationic detergent (e.g. CTAB), which may be used during virion disruption. Removal by β-propiolactone treatment can also be used.

Measurement of residual host cell DNA is now a routine regulatory requirement for biologicals and is within the normal capabilities of the skilled person. The assay used to measure DNA will typically be a validated assay [49,50]. The performance characteristics of a validated assay can be described in mathematical and quantifiable terms, and its possible sources of error will have been identified. The assay will generally have been tested for characteristics such as accuracy, precision, specificity. Once an assay has been calibrated (e.g. against known standard quantities of host cell DNA) and tested then quantitative DNA measurements can be routinely performed. Three main techniques for DNA quantification can be used: hybridization methods, such as Southern blots or slot blots [51]; immunoassay methods, such as the Threshold™ System [52]; and quantitative PCR [53]. These methods are all familiar to the skilled person, although the precise characteristics of each method may depend on the host cell in question e.g. the choice of probes for hybridization, the choice of primers and/or probes for amplification, etc. The Threshold™ system from Molecular Devices is a quantitative assay for picogram levels of total DNA, and has been used for monitoring levels of contaminating DNA in biopharmaceuticals [52]. A typical assay involves non-sequence-specific formation of a reaction complex between a biotinylated ssDNA binding protein, a urease-conjugated anti-ssDNA antibody, and DNA. All assay components are included in the complete Total DNA Assay Kit available from the manufacturer. Various commercial manufacturers offer quantitative PCR assays for detecting residual host cell DNA e.g. AppTec™ Laboratory Services, BioReliance™, Althea Technologies, etc. A comparison of a chemiluminescent hybridisation assay and the total DNA Threshold™ system for measuring host cell DNA contamination of a human viral vaccine can be found in reference 54.

An influenza virus from which immunogens are prepared may be a wild-type strain or, more typically, a reassortant strain. Such reassortant strains may have been obtained by reverse genetics techniques. Reverse genetics techniques [e.g. 55-59] allow influenza viruses with desired genome segments to be prepared in vitro using expression constructs such as plasmids. Typically, they involve expressing (a) DNA molecules that encode desired viral RNA molecules e.g. from poll promoters, and (b) DNA molecules that encode viral proteins e.g. from poIII promoters, such that expression of both types of DNA in a cell leads to assembly of a complete intact infectious virion. The DNA preferably provides all of the viral RNA and proteins, but it is also possible to use a helper virus to provide some of the RNA and proteins. Plasmid-based methods using separate plasmids for producing each viral RNA are preferred [60-62], and these methods will also involve the use of plasmids to express all or some (e.g. just the PB1, PB2, PA and NP proteins) of the viral proteins, with up to 12 plasmids being used in some methods. To reduce the number of plasmids needed, one approach [63] combines a plurality of RNA polymerase I transcription cassettes (for viral RNA synthesis) on the same plasmid (e.g. sequences encoding 1, 2, 3, 4, 5, 6, 7 or all 8 influenza A vRNA segments), and a plurality of protein-coding regions with RNA polymerase II promoters on another plasmid (e.g. sequences encoding 1, 2, 3, 4, 5, 6, 7 or all 8 influenza A mRNA transcripts). Preferred aspects of the reference 63 method involve: (a) PB1, PB2 and PA mRNA-encoding regions on a single plasmid; and (b) all 8 vRNA-encoding segments on a single plasmid. It is possible to use dual poll and poIII promoters to simultaneously code for the viral RNAs and for expressible mRNAs from a single template [64,65].

Thus the virus may include one or more RNA segments from a A/PR/8/34 virus (typically 6 segments from A/PR/8/34, with the HA and N segments being from a vaccine strain, i.e. a 6:2 reassortant), particularly when viruses are grown in eggs. It may also include one or more RNA segments from a A/WSN/33 virus, or from any other virus strain useful for generating reassortant viruses for vaccine preparation. Typically, the invention protects against a strain that is capable of human-to-human transmission, and so the strain's genome will usually include at least one RNA segment that originated in a mammalian (e.g. in a human) influenza virus. It may include NS segment that originated in an avian influenza virus.

### The Pneumococcal Immunogen

The pneumococcal immunogen can take various forms. For instance, it may comprise a capsular saccharide and/or a polypeptide from a pneumococcus. In preferred embodiments the pneumococcal immunogen comprises at least one pneumococcal polypeptide. Current pneumococcal vaccines are based on capsular saccharides, either conjugated to a carrier protein or in unconjugated form. The pneumococcal immunogen can comprise one or more such capsular saccharides, but in some embodiments the pneumococcal immunogen comprises no pneumococcal capsular saccharide. Where it is present, the saccharide may be a polysaccharide having the size that arises during purification of the saccharide from bacteria, or it may be an oligosaccharide achieved by fragmentation of such a polysaccharide. In the 7-valent PREVNAR™ product, for instance, 6 of the saccharides are presented as intact polysaccharides while one (the 18C serotype) is presented as an oligosaccharide.

A pneumococcal immunogen may comprise a capsular saccharide from one or more of the following pneumococcal serotypes: 1, 2, 3, 4, 5, 6A, 6B, 7F, 8, 9N, 9V, 10A, 11A, 12F, 14, 15B, 17F, 18C, 19A, 19F, 20, 22F, 23F and/or 33F. An immunogen may include saccharide from multiple serotypes e.g. 2, 3, 4, 5, 6, 7, 8, 9, 10, 11, 12, 13, 14, 15, 16, 17, 18, 19, 20, 21, 22, 23 or more different serotypes. 7-valent, 9-valent, 10-valent, 11-valent and 13-valent conjugate combinations are already known in the art, as is a 23-valent unconjugated combination, and any of these may be used with the invention. For example, a 7-valent combination (such as the PREVNAR™ product) may include saccharide from serotypes 4, 6B, 9V, 14, 18C, 19F and 23F. A 10-valent combination (such as the SYNFLORIX™ product) may include saccharide from serotypes 1, 4, 5, 6B, 7F, 9V, 14, 18C, 19F and 23F. An 11-valent combination may further include saccharide from serotype 3. A 12-valent combination may add to the 10-valent mixture: serotypes 6A and 19A; 6A and 22F; 19A and 22F; 6A and 15B; 19A and 15B; r 22F and 15B; A 13-valent combination may add to the 11-valent mixture: serotypes 19A and 22F; 8 and 12F; 8 and 15B; 8 and 19A; 8 and 22F; 12F and 15B; 12F and 19A; 12F and 22F; 15B and 19A; 15B and 22F. etc. One useful 13-valent combination includes capsular saccharide from serotypes 1, 3, 4, 5, 6A, 6B, 7F, 9V, 14, 18C, 19, 19F and 23F. Where more than one serotype is used, it is useful to include 1, 2 or 3 of serotypes 1, 5 and 14.

If a capsular saccharide is used as a pneumococcal immunogen, it is preferably conjugated to a carrier protein. The carrier may be a pneumococcal antigen such as RrgB, spr0057, spr0096 and spr2021, etc., or pneumolysin [66] or its non-toxic derivatives [67], or pneumococcal surface protein PspA [68]. In other embodiments, though, the carrier is not a pneumococcal antigen, and may be e.g. a bacterial toxin or toxoid. Typical carrier proteins are diphtheria or tetanus toxoids or mutants thereof. The CRM₁₉₇ diphtheria toxin mutant [69] is useful, and is the carrier in the PREVNAR™ product. Other suitable carrier proteins include N. meningitidis outer membrane protein complex [70], synthetic peptides [71,72], heat shock proteins [73,74], pertussis proteins [75,76], cytokines [77], lymphokines [77], hormones [77], growth factors [77], artificial proteins comprising multiple human CD4⁺ T cell epitopes from various pathogen-derived antigens [78] such as N19 [79], protein D from H. influenzae [80-82], iron-uptake proteins [83], toxin A or B from C. difficile [84], recombinant P. aeruginosa exoprotein A (rEPA) [85], etc.

Where a composition includes more than one conjugate, each conjugate may use the same carrier protein or a different carrier protein. Reference 86 describes potential advantages when using different carrier proteins in multivalent pneumococcal conjugate vaccines

In some embodiments, a single conjugate may carry saccharides from multiple serotypes [87]. Usually, however, each conjugate will include saccharide from a single serotype.

Conjugates may have excess carrier (w/w) or excess saccharide (w/w). In some embodiments, a conjugate may include equal weights of each.

The carrier molecule may be covalently conjugated to the carrier directly or via a linker. Direct linkages to the protein may be achieved by, for instance, reductive amination between the saccharide and the carrier, as described in, for example, references 88 and 89. The saccharide may first need to be activated e.g. by oxidation. Linkages via a linker group may be made using any known procedure, for example, the procedures described in references 90 and 91. A preferred type of linkage is an adipic acid linker, which may be formed by coupling a free —NH₂ group (e.g. introduced to a glucan by amination) with adipic acid (using, for example, diimide activation), and then coupling a protein to the resulting saccharide-adipic acid intermediate [92,93]. Another preferred type of linkage is a carbonyl linker, which may be formed by reaction of a free hydroxyl group of a saccharide CDI [94, 95] followed by reaction with a protein to form a carbamate linkage. Other linkers include β-propionamido [96], nitrophenyl-ethylamine [97], haloacyl halides [98], glycosidic linkages [99], 6-aminocaproic acid [100], ADH [101], C₄ to C₁₂ moieties [102], etc. Carbodiimide condensation can also be used [103].

A pneumococcal immunogen may comprise one or more of the following pneumococcal polypeptides: (1) a spr0057 antigen; (2) a spr0565 antigen; (3) a spr1098 antigen; (4) a spr1416 antigen; (5) a spr1418 antigen; (6) a spr0867 antigen; (7) a spr1431 antigen; (8) a spr1739 antigen; (9) a spr2021 antigen; (10) a spr0096 antigen; (11) a spr1707 antigen; (12) a spr1875 antigen; (13) a spr0884 antigen; and/or (14) a RrgB antigen. Similarly, a pneumococcal immunogen may comprise one or more of the following pneumococcal polypeptides: (1) ClpP; (2) LytA; (3) PhtA; (4) PhtB; (5) PhtD; (6) PhtE; (7) ZmpB; (8) CbpD; (9) CbpG; (10) PvaA; (11) CPL1; (12) PspC; (13) PspA; (14) PsaA; (15) PrtA; (16) Sp133; (17) PiaA; (18) PiuA; (19) CbiO; and/or (20) 30S ribosomal protein S8. These antigens may be present as separate polypeptides, or they may be present as fusion polypeptides e.g. a spr0057-spr0096 fusion or a spr0096-spr2021 fusion, a spr0565-PhtD fusion, a RrgB-spr0057 fusion, etc.

The original ‘spr0057’ sequence was annotated in reference 104 as ‘Beta-N-acetyl-hexosaminidase precursor’ (see GI:15902101). For reference purposes, the amino acid sequence of full length spr0057 as found in the R6 strain is given as SEQ ID NO: 23 herein. Preferred spr0057 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 23; and/or (b) comprising a fragment of at least ‘n’ consecutive amino acids of SEQ ID NO: 23, wherein ‘n’ is 7 or more (e.g. 8, 10, 12, 14, 16, 18, 20, 25, 30, 35, 40, 50, 60, 70, 80, 90, 100, 150, 200, 250 or more). These spr0057 proteins include variants of SEQ ID NO: 23. Preferred fragments of (b) comprise an epitope from SEQ ID NO: 23. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 23 while retaining at least one epitope of SEQ ID NO: 23. Other fragments omit one or more protein domains. One suitable fragment is SEQ ID NO: 38, which omits the natural leader peptide and sortase recognition sequences. Another suitable fragment is SEQ ID NO: 24, which has N-terminal and C-terminal truncations. SEQ ID NO: 27 is a variant of SEQ ID NO: 24 based on a different wild-type strain and is a useful spr0057 sequence for use with the invention.

The original ‘spr0565’ sequence was annotated in reference 104 as ‘beta-galactosidase precursor’ (see GI:15902609). For reference purposes, the amino acid sequence of full length spr0565 as found in the R6 strain is given as SEQ ID NO: 25 herein. Preferred spr0565 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 25; and/or (b) comprising a fragment of at least ‘n’ consecutive amino acids of SEQ ID NO: 25, wherein ‘n’ is 7 or more (e.g. 8, 10, 12, 14, 16, 18, 20, 25, 30, 35, 40, 50, 60, 70, 80, 90, 100, 150, 200, 250 or more). These spr0565 proteins include variants of SEQ ID NO: 25 (e.g. SEQ ID NO: 45; see below). Preferred fragments of (b) comprise an epitope from SEQ ID NO: 25. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 25 while retaining at least one epitope of SEQ ID NO: 25. Other fragments omit one or more protein domains. One suitable fragment is SEQ ID NO: 42, which omits the natural leader peptide and sortase recognition sequences. Other suitable fragments are SEQ ID NOs: 43 and 44. These shortened versions of spr0565 are particularly useful because the natural polypeptide is very long (>2000 aa). A variant form of spr0565 is SEQ ID NO: 45 herein. The use of this variant form for immunisation is reported in reference 105 (SEQ ID NO: 178 therein). Useful spr0565 polypeptides may thus comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 45; and/or (b) comprising a fragment of at least ‘n’ consecutive amino acids of SEQ ID NO: 45, wherein ‘n’ is 7 or more (e.g. 8, 10, 12, 14, 16, 18, 20, 25, 30, 35, 40, 50, 60, 70, 80, 90, 100, 150, 200, 250 or more). These polypeptides include variants of SEQ ID NO: 45. Preferred fragments of (b) comprise an epitope from SEQ ID NO: 45. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 45 while retaining at least one epitope of SEQ ID NO: 45. Other fragments omit one or more protein domains. Immunogenic fragments of SEQ ID NO: 45 are identified in table 1 of reference 105.

The original ‘spr1098’ sequence was annotated in reference 104 as ‘Sortase’ (see GI:15903141). For reference purposes, the amino acid sequence of full length spr1098 as found in the R6 strain is given as SEQ ID NO: 26 herein. Preferred spr1098 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 26; and/or (b) comprising a fragment of at least ‘n’ consecutive amino acids of SEQ ID NO: 26, wherein ‘n’ is 7 or more (e.g. 8, 10, 12, 14, 16, 18, 20, 25, 30, 35, 40, 50, 60, 70, 80, 90, 100, 150, 200, 250 or more). These spr1098 proteins include variants of SEQ ID NO: 26. Preferred fragments of (b) comprise an epitope from SEQ ID NO: 26. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 26 while retaining at least one epitope of SEQ ID NO: 26. Other fragments omit one or more protein domains. One suitable fragment is SEQ ID NO: 46, which omits the natural leader peptide sequence.

The original ‘spr1416’ sequence was annotated in reference 104 as ‘hypothetical protein’ (see GI:15903459). For reference purposes, the amino acid sequence of full length spr1416 as found in the R6 strain is given as SEQ ID NO: 28 herein. Preferred spr1416 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 28; and/or (b) comprising a fragment of at least ‘n’ consecutive amino acids of SEQ ID NO: 28, wherein ‘n’ is 7 or more (e.g. 8, 10, 12, 14, 16, 18, 20, 25, 30, 35, 40, 50, 60, 70, 80, 90, 100, 150, 200, 250 or more). These spr1416 proteins include variants of SEQ ID NO: 28. Preferred fragments of (b) comprise an epitope from SEQ ID NO: 28. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 28 while retaining at least one epitope of SEQ ID NO: 28. Other fragments omit one or more protein domains.

The original ‘spr1418’ sequence was annotated in reference 104 as ‘hypothetical protein’ (see GI:15903461). For reference purposes, the amino acid sequence of full length spr1418 as found in the R6 strain is given as SEQ ID NO: 29 herein. Preferred spr1418 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 29; and/or (b) comprising a fragment of at least ‘n’ consecutive amino acids of SEQ ID NO: 29, wherein ‘n’ is 7 or more (e.g. 8, 10, 12, 14, 16, 18, 20, 25, 30, 35, 40, 50, 60, 70, 80, 90, 100, 150, 200, 250 or more). These spr1418 proteins include variants of SEQ ID NO: 29. Preferred fragments of (b) comprise an epitope from SEQ ID NO: 29. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 29 while retaining at least one epitope of SEQ ID NO: 29. Other fragments omit one or more protein domains.

The original ‘spr0867’ sequence was annotated in reference 104 as ‘Endo-beta-N-acetylglucosaminidase’ (see GI:15902911). For reference purposes, the amino acid sequence of full length spr0867 as found in the R6 strain is given as SEQ ID NO: 30 herein. Preferred spr0867 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 30; and/or (b) comprising a fragment of at least ‘n’ consecutive amino acids of SEQ ID NO: 30, wherein ‘n’ is 7 or more (e.g. 8, 10, 12, 14, 16, 18, 20, 25, 30, 35, 40, 50, 60, 70, 80, 90, 100, 150, 200, 250 or more). These spr0867 proteins include variants of SEQ ID NO: 30. Preferred fragments of (b) comprise an epitope from SEQ ID NO: 30. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 30 while retaining at least one epitope of SEQ ID NO: 30. Other fragments omit one or more protein domains. One suitable fragment is SEQ ID NO: 48, which omits the natural leader peptide sequence.

The original ‘spr1431’ sequence was annotated in reference 104 as ‘1,4-beta-N-acetylmuramidase’ (see GI:15903474). It is also known as ‘LytC’, and its use for immunisation is reported in reference 126. For reference purposes, the amino acid sequence of full length spr1431 as found in the R6 strain is given as SEQ ID NO: 31 herein. Preferred spr1431 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 31; and/or (b) comprising a fragment of at least ‘n’ consecutive amino acids of SEQ ID NO: 31, wherein ‘n’ is 7 or more (e.g. 8, 10, 12, 14, 16, 18, 20, 25, 30, 35, 40, 50, 60, 70, 80, 90, 100, 150, 200, 250 or more). These spr1431 proteins include variants of SEQ ID NO: 31. Preferred fragments of (b) comprise an epitope from SEQ ID NO: 31. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 31 while retaining at least one epitope of SEQ ID NO: 31. Other fragments omit one or more protein domains. One suitable fragment is SEQ ID NO: 49, which omits the natural leader peptide sequence.

The ‘spr1739’ polypeptide is pneumolysin (e.g. see GI:15903781). For reference purposes, the amino acid sequence of full length spr1739 as found in the R6 strain is given as SEQ ID NO: 32 herein. Preferred spr1739 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 32; and/or (b) comprising a fragment of at least ‘n’ consecutive amino acids of SEQ ID NO: 32, wherein ‘n’ is 7 or more (e.g. 8, 10, 12, 14, 16, 18, 20, 25, 30, 35, 40, 50, 60, 70, 80, 90, 100, 150, 200, 250 or more). These spr1739 proteins include variants of SEQ ID NO: 32. Preferred fragments of (b) comprise an epitope from SEQ ID NO: 32. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 32 while retaining at least one epitope of SEQ ID NO: 32. Other fragments omit one or more protein domains. Mutant forms of pneumolysin for vaccination use are known in the art [67, 106-111], and these mutant forms may be used with the invention. Detoxification can be achieved by C-terminal truncation (e.g. see ref. 112) e.g. deleting 34 amino acids, 45 amino acids, 7 amino acids [113], etc. Further mutations, numbered according to SEQ ID NO: 32, include Pro325→Leu (e.g. SEQ ID NO: 50) and/or Trp433→Phe (e.g. SEQ ID NO: 51). These mutations may be combined with C-terminal truncations e.g. to combine a Pro325→Leu mutation with a 7-mer truncation (e.g. SEQ ID NO: 52).

The original ‘spr2021’ sequence was annotated in reference 104 as ‘General stress protein GSP-781’ (see GI:15904062). For reference purposes, the amino acid sequence of full length spr2021 as found in the R6 strain is given as SEQ ID NO: 33 herein. Preferred spr2021 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 33; and/or (b) comprising a fragment of at least ‘n’ consecutive amino acids of SEQ ID NO: 33, wherein ‘n’ is 7 or more (e.g. 8, 10, 12, 14, 16, 18, 20, 25, 30, 35, 40, 50, 60, 70, 80, 90, 100, 150, 200, 250 or more). These spr2021 proteins include variants of SEQ ID NO: 33. Preferred fragments of (b) comprise an epitope from SEQ ID NO: 33. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 33 while retaining at least one epitope of SEQ ID NO: 33. Other fragments omit one or more protein domains. One suitable fragment is SEQ ID NO: 53, which omits the natural leader peptide sequence. Reference 105 annotates spr2021 as a secreted 45 kDa protein with homology to GbpB and discloses its use as an immunogen (SEQ ID NO: 243 therein; SP2216). Immunogenic fragments of spr2021 are identified in table 1 of reference 105 (page 73). Another useful fragment of spr2021 is disclosed as SEQ ID NO: 1 of reference 114 (amino acids 28-278 of SEQ ID NO: 33 herein).

The original ‘spr0096’ sequence was annotated in reference 104 as ‘hypothetical protein’ (see GI:15902140). For reference purposes, the amino acid sequence of full length spr0096 as found in the R6 strain is given as SEQ ID NO: 34 herein. Preferred spr0096 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 34; and/or (b) comprising a fragment of at least ‘n’ consecutive amino acids of SEQ ID NO: 34, wherein ‘n’ is 7 or more (e.g. 8, 10, 12, 14, 16, 18, 20, 25, 30, 35, 40, 50, 60, 70, 80, 90, 100, 150, 200, 250 or more). These spr0096 proteins include variants of SEQ ID NO: 34 (e.g. SEQ ID NO: 40). Preferred fragments of (b) comprise an epitope from SEQ ID NO: 34. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 34 while retaining at least one epitope of SEQ ID NO: 34. Other fragments omit one or more protein domains. A variant form of spr0096, with an insert near its C-terminus relative to SEQ ID NO: 34, is SEQ ID NO: 54 herein. The use of this variant for immunisation is reported in reference 105 (SEQ ID NO: 150 therein), where it is annotated as a LysM domain protein. Thus a spr0096 for use with the invention may comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 54; and/or (b) comprising a fragment of at least ‘n’ consecutive amino acids of SEQ ID NO: 54, wherein ‘n’ is 7 or more (e.g. 8, 10, 12, 14, 16, 18, 20, 25, 30, 35, 40, 50, 60, 70, 80, 90, 100, 150, 200, 250 or more). These polypeptides include variants of SEQ ID NO: 54. Preferred fragments of (b) comprise an epitope from SEQ ID NO: 54. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 54 while retaining at least one epitope of SEQ ID NO: 54. Other fragments omit one or more protein domains. Immunogenic fragments of SEQ ID NO: 54 are identified in table 1 of reference 105. A spr0096 polypeptide may be used in the form of a dimer e.g. a homodimer.

The original ‘spr1707’ sequence was annotated in reference 104 as ‘ABC transporter substrate-binding protein-oligopeptide transport’ (see GI:15903749). For reference purposes, the amino acid sequence of full length spr1707 as found in the R6 strain is given as SEQ ID NO: 36 herein.

Preferred spr1707 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 36; and/or (b) comprising a fragment of at least ‘n’ consecutive amino acids of SEQ ID NO: 36, wherein ‘n’ is 7 or more (e.g. 8, 10, 12, 14, 16, 18, 20, 25, 30, 35, 40, 50, 60, 70, 80, 90, 100, 150, 200, 250 or more). These spr1707 proteins include variants of SEQ ID NO: 36 (e.g. SEQ ID NO: 100; see below). Preferred fragments of (b) comprise an epitope from SEQ ID NO: 36. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 36 while retaining at least one epitope of SEQ ID NO: 36. Other fragments omit one or more protein domains. A variant form of spr1707, differing from SEQ ID NO: 36 by 4 amino acids, is SEQ ID NO: 55 herein. The use of SEQ ID NO: 55 for immunisation is reported in reference 105 (SEQ ID NO: 220 therein). Thus a spr1707 polypeptide for use with the invention may comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 55; and/or (b) comprising a fragment of at least ‘n’ consecutive amino acids of SEQ ID NO: 55, wherein ‘n’ is 7 or more (e.g. 8, 10, 12, 14, 16, 18, 20, 25, 30, 35, 40, 50, 60, 70, 80, 90, 100, 150, 200, 250 or more). These polypeptides include variants of SEQ ID NO: 55. Preferred fragments of (b) comprise an epitope from SEQ ID NO: 55. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 55 while retaining at least one epitope of SEQ ID NO: 55. Other fragments omit one or more protein domains. Immunogenic fragments of SEQ ID NO: 55 are identified in table 1 of reference 105.

The original ‘spr1875’ sequence was annotated in reference 104 as ‘hypothetical protein’ (see GI:15903916). For reference purposes, the amino acid sequence of full length spr1875 as found in the R6 strain is given as SEQ ID NO: 35 herein. Preferred spr1875 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 35; and/or (b) comprising a fragment of at least ‘n’ consecutive amino acids of SEQ ID NO: 35, wherein ‘n’ is 7 or more (e.g. 8, 10, 12, 14, 16, 18, 20, 25, 30, 35, 40, 50, 60, 70, 80, 90, 100, 150, 200, 250 or more). These spr1875 proteins include variants of SEQ ID NO: 35. Preferred fragments of (b) comprise an epitope from SEQ ID NO: 35. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 35 while retaining at least one epitope of SEQ ID NO: 35. Other fragments omit one or more protein domains.

The ‘spr0884’ protein is a peptidylprolyl isomerase, also known as protease maturation protein. For reference purposes, the amino acid sequence of full length spr0884 is SEQ ID NO: 37 herein. Preferred spr0884 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 37; and/or (b) comprising a fragment of at least ‘n’ consecutive amino acids of SEQ ID NO: 37, wherein ‘n’ is 7 or more (e.g. 8, 10, 12, 14, 16, 18, 20, 25, 30, 35, 40, 50, 60, 70, 80, 90, 100, 150, 200, 250 or more). These spr0884 proteins include variants of SEQ ID NO: 37. Preferred fragments of (b) comprise an epitope from SEQ ID NO: 37. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 37 while retaining at least one epitope of SEQ ID NO: 37. Other fragments omit one or more protein domains. One suitable fragment is SEQ ID NO: 56, which omits the natural leader peptide sequence. The use of spr0884 for immunisation is reported in reference 115.

ClpP is the ATP-dependent Clp protease proteolytic subunit. For reference purposes, the amino acid sequence of full length ClpP is SEQ ID NO: 58 herein. In the R6 genome ClpP is spr0656 [104]. Preferred ClpP polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 58; and/or (b) comprising a fragment of at least ‘n’ consecutive amino acids of SEQ ID NO: 58, wherein ‘n’ is 7 or more (e.g. 8, 10, 12, 14, 16, 18, 20, 25, 30, 35, 40, 50, 60, 70, 80, 90, 100, 150, 200, 250 or more). These ClpP proteins include variants of SEQ ID NO: 58. Preferred fragments of (b) comprise an epitope from SEQ ID NO: 58. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 58 while retaining at least one epitope of SEQ ID NO: 58. Other fragments omit one or more protein domains. The use of ClpP for immunisation is reported in references 116 and 117. It may advantageously be used in combination with PspA and PsaA and/or PspC [116].

LytA is the N-acetylmuramoyl-L-alanine amidase (autolysin). For reference purposes, the amino acid sequence of full length LytA is SEQ ID NO: 59 herein. In the R6 genome LytA is spr1754 [104]. Preferred LytA polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 59; and/or (b) comprising a fragment of at least ‘n’ consecutive amino acids of SEQ ID NO: 59, wherein ‘n’ is 7 or more (e.g. 8, 10, 12, 14, 16, 18, 20, 25, 30, 35, 40, 50, 60, 70, 80, 90, 100, 150, 200, 250 or more). These LytA proteins include variants of SEQ ID NO: 59 (e.g. GI:18568354). Preferred fragments of (b) comprise an epitope from SEQ ID NO: 59. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 59 while retaining at least one epitope of SEQ ID NO: 59. Other fragments omit one or more protein domains. The use of LytA for immunisation is reported in reference 118, particularly in a form comprising the LytA choline binding domain fused to a heterologous promiscuous T helper epitope.

PhtA is the Pneumococcal histidine triad protein A. For reference purposes, the amino acid sequence of full length PhtA precursor is SEQ ID NO: 60 herein. In the R6 genome PhtA is spr1061 [104]. Preferred PhtA polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 60; and/or (b) comprising a fragment of at least ‘n’ consecutive amino acids of SEQ ID NO: 60, wherein ‘n’ is 7 or more (e.g. 8, 10, 12, 14, 16, 18, 20, 25, 30, 35, 40, 50, 60, 70, 80, 90, 100, 150, 200, 250 or more). These PhtA proteins include variants of SEQ ID NO: 60. Preferred fragments of (b) comprise an epitope from SEQ ID NO: 60. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 60 while retaining at least one epitope of SEQ ID NO: 60. Other fragments omit one or more protein domains. The use of PhtA for immunisation is reported in references 119 and 120.

PhtB is the pneumococcal histidine triad protein B. For reference purposes, the amino acid sequence of full length PhtB precursor is SEQ ID NO: 61 herein. Xaa at residue 578 can be Lysine. Preferred PhtB polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 61; and/or (b) comprising a fragment of at least ‘n’ consecutive amino acids of SEQ ID NO: 61, wherein ‘n’ is 7 or more (e.g. 8, 10, 12, 14, 16, 18, 20, 25, 30, 35, 40, 50, 60, 70, 80, 90, 100, 150, 200, 250 or more). These PhtB proteins include variants of SEQ ID NO: 61. Preferred fragments of (b) comprise an epitope from SEQ ID NO: 61. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 61 while retaining at least one epitope of SEQ ID NO: 61. Other fragments omit one or more protein domains. The use of PhtB for immunisation is reported in references 119, 120 and 121.

PhtD is the Pneumococcal histidine triad protein D. For reference purposes, the amino acid sequence of full length PhtD precursor is SEQ ID NO: 62 herein. In the R6 genome PhtD is spr0907 [104]. Preferred PhtD polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 62; and/or (b) comprising a fragment of at least ‘n’ consecutive amino acids of SEQ ID NO: 62, wherein ‘n’ is 7 or more (e.g. 8, 10, 12, 14, 16, 18, 20, 25, 30, 35, 40, 50, 60, 70, 80, 90, 100, 150, 200, 250 or more). These PhtD proteins include variants of SEQ ID NO: 62. Preferred fragments of (b) comprise an epitope from SEQ ID NO: 62.

Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 62 while retaining at least one epitope of SEQ ID NO: 62. Other fragments omit one or more protein domains. The use of PhtD for immunisation is reported in references 119, 120 and 122.

PhtE is the Pneumococcal histidine triad protein E. For reference purposes, the amino acid sequence of full length PhtE precursor is SEQ ID NO: 63 herein. In the R6 genome PhtE is spr0908 [104]. Preferred PhtE polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 63; and/or (b) comprising a fragment of at least ‘n’ consecutive amino acids of SEQ ID NO: 63, wherein ‘n’ is 7 or more (e.g. 8, 10, 12, 14, 16, 18, 20, 25, 30, 35, 40, 50, 60, 70, 80, 90, 100, 150, 200, 250 or more). These PhtE proteins include variants of SEQ ID NO: 63. Preferred fragments of (b) comprise an epitope from SEQ ID NO: 63. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 63 while retaining at least one epitope of SEQ ID NO: 63. Other fragments omit one or more protein domains. The use of PhtE for immunisation is reported in references 119 and 120.

ZmpB is the zinc metalloprotease. For reference purposes, the amino acid sequence of full length ZmpB is SEQ ID NO: 64 herein. In the R6 genome ZmpB is spr0581 [104]. Preferred ZmpB polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 64; and/or (b) comprising a fragment of at least ‘n’ consecutive amino acids of SEQ ID NO: 64, wherein ‘n’ is 7 or more (e.g. 8, 10, 12, 14, 16, 18, 20, 25, 30, 35, 40, 50, 60, 70, 80, 90, 100, 150, 200, 250 or more). These ZmpB proteins include variants of SEQ ID NO: 64. Preferred fragments of (b) comprise an epitope from SEQ ID NO: 64. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 64 while retaining at least one epitope of SEQ ID NO: 64. Other fragments omit one or more protein domains.

CbpD is the Choline binding protein D. For reference purposes, the amino acid sequence of full length CbpD is SEQ ID NO: 65 herein. In the R6 genome CbpD is spr2006 [104]. Preferred CbpD polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 65; and/or (b) comprising a fragment of at least ‘n’ consecutive amino acids of SEQ ID NO: 65, wherein ‘n’ is 7 or more (e.g. 8, 10, 12, 14, 16, 18, 20, 25, 30, 35, 40, 50, 60, 70, 80, 90, 100, 150, 200, 250 or more). These CbpD proteins include variants of SEQ ID NO: 65 (e.g. SEQ ID NO: 57; see below). Preferred fragments of (b) comprise an epitope from SEQ ID NO: 65. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 65 while retaining at least one epitope of SEQ ID NO: 65. Other fragments omit one or more protein domains. The use of CbpD for immunisation is reported in reference 126. A variant of SEQ ID NO: 65 is SEQ ID NO: 57 herein. The use of SEQ ID NO: 57 for immunisation is reported in reference 105 (SEQ ID NO: 241 therein). Thus a CbpD polypeptide for use with the invention may comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 57; and/or (b) comprising a fragment of at least ‘n’ consecutive amino acids of SEQ ID NO: 57, wherein ‘n’ is 7 or more (e.g. 8, 10, 12, 14, 16, 18, 20, 25, 30, 35, 40, 50, 60, 70, 80, 90, 100, 150, 200, 250 or more). These CbpD proteins include variants of SEQ ID NO: 57. Preferred fragments of (b) comprise an epitope from SEQ ID NO: 57. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 57 while retaining at least one epitope of SEQ ID NO: 57. Other fragments omit one or more protein domains. Immunogenic fragments of SEQ ID NO: 57 are identified in table 1 of ref.105.

CbpG is the Choline binding protein G. For reference purposes, the amino acid sequence of full length CbpG is SEQ ID NO: 47 herein. In the R6 genome CbpG is spr0350 [104]. Preferred CbpG polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 47; and/or (b) comprising a fragment of at least ‘n’ consecutive amino acids of SEQ ID NO: 47, wherein ‘n’ is 7 or more (e.g. 8, 10, 12, 14, 16, 18, 20, 25, 30, 35, 40, 50, 60, 70, 80, 90, 100, 150, 200, 250 or more). These CbpG proteins include variants of SEQ ID NO: 47. Preferred fragments of (b) comprise an epitope from SEQ ID NO: 47. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 47 while retaining at least one epitope of SEQ ID NO: 47. Other fragments omit one or more protein domains. The use of CbpG for immunisation is reported in reference 126.

PvaA (Streptococcus pneumoniae pneumococcal vaccine antigen A) is also known as sp101. For reference purposes, the amino acid sequence of full length PvaA is SEQ ID NO: 41 herein. In the R6 genome PvaA is spr0930 [104]. Preferred PvaA polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 41; and/or (b) comprising a fragment of at least ‘n’ consecutive amino acids of SEQ ID NO: 41, wherein ‘n’ is 7 or more (e.g. 8, 10, 12, 14, 16, 18, 20, 25, 30, 35, 40, 50, 60, 70, 80, 90, 100, 150, 200, 250 or more). These PvaA proteins include variants of SEQ ID NO: 41. Preferred fragments of (b) comprise an epitope from SEQ ID NO: 41. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 41 while retaining at least one epitope of SEQ ID NO: 41. Other fragments omit one or more protein domains. The use of PvaA for immunisation is reported in references 123 and 124.

CPL1 is the pneumococcal phage CPI lysozyme. For reference purposes, the amino acid sequence of full length CPL1 is SEQ ID NO: 39 herein. Preferred CPL1 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 39; and/or (b) comprising a fragment of at least ‘n’ consecutive amino acids of SEQ ID NO: 39, wherein ‘n’ is 7 or more (e.g. 8, 10, 12, 14, 16, 18, 20, 25, 30, 35, 40, 50, 60, 70, 80, 90, 100, 150, 200, 250 or more). These CPL1 proteins include variants of SEQ ID NO: 39. Preferred fragments of (b) comprise an epitope from SEQ ID NO: 39. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 39 while retaining at least one epitope of SEQ ID NO: 39. Other fragments omit one or more protein domains. The use of CPL1 for immunisation is reported in reference 118, particularly in a form comprising the CPL1 choline binding domain fused to a heterologous promiscuous T helper epitope.

PspC is the pneumococcal surface protein C [125] and is also known as choline-binding protein A (CbpA). Its use for immunisation is reported in references 123 and 126. In the R6 strain it is spr1995 and, for reference, the amino acid sequence of full length spr1995 is SEQ ID NO: 22 herein. Preferred PspC polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 22; and/or (b) comprising a fragment of at least ‘n’ consecutive amino acids of SEQ ID NO: 22, wherein ‘n’ is 7 or more (e.g. 8, 10, 12, 14, 16, 18, 20, 25, 30, 35, 40, 50, 60, 70, 80, 90, 100, 150, 200, 250 or more). These spr1995 proteins include variants of SEQ ID NO: 22 (e.g. SEQ ID NO: 20; see below). Preferred fragments of (b) comprise an epitope from SEQ ID NO: 22. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 22 while retaining at least one epitope of SEQ ID NO: 22. Other fragments omit one or more protein domains.

A variant of PspC is known as ‘Hic’. It is similar to PspC, as shown in FIG. 1 of reference 127, where it is reported to bind to factor H (1H). For reference purposes, the amino acid sequence of full length Hic is SEQ ID NO: 20 herein. A Hic protein may be used with the invention in addition to or in place of a PspC polypeptide. Preferred Hic polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 20; and/or (b) comprising a fragment of at least ‘n’ consecutive amino acids of SEQ ID NO: 20, wherein ‘n’ is 7 or more (e.g. 8, 10, 12, 14, 16, 18, 20, 25, 30, 35, 40, 50, 60, 70, 80, 90, 100, 150, 200, 250 or more). These Hic proteins include variants of SEQ ID NO: 20. Preferred fragments of (b) comprise an epitope from SEQ ID NO: 20. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 20 while retaining at least one epitope of SEQ ID NO: 20. Other fragments omit one or more protein domains. PspC and/or Hic can advantageously be used in combination with PspA and/or PsaA.

PspA is the Pneumococcal surface protein A. For reference purposes, the amino acid sequence of full length PspA is SEQ ID NO: 18 herein. In the R6 genome PspA is spr0121 [104]. Preferred PspA polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 18; and/or (b) comprising a fragment of at least ‘n’ consecutive amino acids of SEQ ID NO: 18, wherein ‘n’ is 7 or more (e.g. 8, 10, 12, 14, 16, 18, 20, 25, 30, 35, 40, 50, 60, 70, 80, 90, 100, 150, 200, 250 or more). These PspA proteins include variants of SEQ ID NO: 18. Preferred fragments of (b) comprise an epitope from SEQ ID NO: 18. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 18 while retaining at least one epitope of SEQ ID NO: 18. Other fragments omit one or more protein domains. The use of PspA for immunisation is reported inter alia in reference 128. It can advantageously be administered in combination with PspC.

PsaA is the Pneumococcal surface adhesin. For reference purposes, the amino acid sequence of full length PsaA is SEQ ID NO: 16 herein. Preferred PsaA polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 16; and/or (b) comprising a fragment of at least ‘n’ consecutive amino acids of SEQ ID NO: 16, wherein ‘n’ is 7 or more (e.g. 8, 10, 12, 14, 16, 18, 20, 25, 30, 35, 40, 50, 60, 70, 80, 90, 100, 150, 200, 250 or more). These PsaA proteins include variants of SEQ ID NO: 16. Preferred fragments of (b) comprise an epitope from SEQ ID NO: 16. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 16 while retaining at least one epitope of SEQ ID NO: 16. Other fragments omit one or more protein domains. A useful fragment of PsaA is disclosed as SEQ ID NO: 3 in reference 114 (corresponding to amino acids 21-309 of SEQ ID NO: 16 herein). The use of PsaA for immunisation is reported in reference 129. It can be used in combination with PspA and/or PspC.

PrtA is the cell wall-associated serine proteinase. It has also been known as sp128 and sp130, and is in a subtilisin-like serine protease. For reference purposes, the amino acid sequence of full length PrtA precursor is SEQ ID NO: 14 herein. In the R6 genome PrtA is spr0561 [104]. Preferred PrtA polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 14; and/or (b) comprising a fragment of at least ‘n’ consecutive amino acids of SEQ ID NO: 14, wherein ‘n’ is 7 or more (e.g. 8, 10, 12, 14, 16, 18, 20, 25, 30, 35, 40, 50, 60, 70, 80, 90, 100, 150, 200, 250 or more). These PrtA proteins include variants of SEQ ID NO: 14. Preferred fragments of (b) comprise an epitope from SEQ ID NO: 14. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 14 while retaining at least one epitope of SEQ ID NO: 14. Other fragments omit one or more protein domains. The use of PrtA for immunisation is reported in references 130 & 131, and also in reference 123.

Sp133 is a conserved pneumococcal antigen. For reference purposes, the amino acid sequence of full length Sp133 is SEQ ID NO: 12 herein. In the R6 genome Sp133 is spr0931 [104]. Preferred Sp133 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 12; and/or (b) comprising a fragment of at least ‘n’ consecutive amino acids of SEQ ID NO: 12, wherein ‘n’ is 7 or more (e.g. 8, 10, 12, 14, 16, 18, 20, 25, 30, 35, 40, 50, 60, 70, 80, 90, 100, 150, 200, 250 or more). These Sp133 proteins include variants of SEQ ID NO: 12. Preferred fragments of (b) comprise an epitope from SEQ ID NO: 12. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 12 while retaining at least one epitope of SEQ ID NO: 12. Other fragments omit one or more protein domains. The use of Sp133 for immunisation is reported in reference 132.

PiaA is the membrane permease involved in iron acquisition by pneumococcus. For reference purposes, the amino acid sequence of full length PiaA is SEQ ID NO: 10 herein. In the R6 genome PiaA is spr0935 [104]. Preferred PiaA polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 10; and/or (b) comprising a fragment of at least ‘n’ consecutive amino acids of SEQ ID NO: 10, wherein ‘n’ is 7 or more (e.g. 8, 10, 12, 14, 16, 18, 20, 25, 30, 35, 40, 50, 60, 70, 80, 90, 100, 150, 200, 250 or more). These PiaA proteins include variants of SEQ ID NO: 10. Preferred fragments of (b) comprise an epitope from SEQ ID NO: 10. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 10 while retaining at least one epitope of SEQ ID NO: 10. Other fragments omit one or more protein domains. The use of PiaA for immunisation is reported in references 133, 134 and 135, particularly in combination with PiuA.

PiuA is the ABC transporter substrate-binding protein for ferric iron transport. It is also known as FatB. For reference purposes, the amino acid sequence of full length PiuA is SEQ ID NO: 9 herein. In the R6 genome PiuA is spr1687 [104]. Preferred PiuA polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 9; and/or (b) comprising a fragment of at least ‘n’ consecutive amino acids of SEQ ID NO: 9, wherein ‘n’ is 7 or more (e.g. 8, 10, 12, 14, 16, 18, 20, 25, 30, 35, 40, 50, 60, 70, 80, 90, 100, 150, 200, 250 or more). These PiuA proteins include variants of SEQ ID NO: 9. Preferred fragments of (b) comprise an epitope from SEQ ID NO: 9. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 9 while retaining at least one epitope of SEQ ID NO: 9. Other fragments omit one or more protein domains. The use of PiuA for immunisation is reported in refs 133 to 135, particularly in combination with PiaA.

CbiO is annotated as a cobalt transporter ATP-binding subunit. For reference purposes, the amino acid sequence of full length CbiO is SEQ ID NO: 8 herein. In the R6 genome CbiO is spr2025 [104]. The use of CbiO for immunisation is reported in reference 136 (‘ID2’ therein). Preferred CbiO polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 8; and/or (b) comprising a fragment of at least ‘n’ consecutive amino acids of SEQ ID NO: 8, wherein ‘n’ is 7 or more (e.g. 8, 10, 12, 14, 16, 18, 20, 25, 30, 35, 40, 50, 60, 70, 80, 90, 100, 150, 200, 250 or more). These CbiO proteins include variants of SEQ ID NO: 8. Preferred fragments of (b) comprise an epitope from SEQ ID NO: 8. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 8 while retaining at least one epitope of SEQ ID NO: 8. Other fragments omit one or more protein domains.

For reference purposes, the amino acid sequence of 30S ribosomal protein S8 is SEQ ID NO: 7 herein. In the R6 genome the S8 subunit is spr0203 [104]. Preferred S8 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 7; and/or (b) comprising a fragment of at least ‘n’ consecutive amino acids of SEQ ID NO: 7, wherein ‘n’ is 7 or more (e.g. 8, 10, 12, 14, 16, 18, 20, 25, 30, 35, 40, 50, 60, 70, 80, 90, 100, 150, 200, 250 or more). These S8 proteins include variants of SEQ ID NO: 7. Preferred fragments of (b) comprise an epitope from SEQ ID NO: 7. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 7 while retaining at least one epitope of SEQ ID NO: 7. Other fragments omit one or more protein domains.

S. pneumoniae has a pilus known as pilus-1 encoded by a 14-kb islet (PI-1) having seven genes encoding: the RlrA transcriptional regulator, three pilus subunits with LPXTG-type cell wall sorting signals, and three sortase enzymes. RrgB is the major subunit that forms the backbone of the structure [137-140]. The RrgB subunit can be used as a pneumococcal immunogen with the invention. It has at least three clades. Reference amino acid sequences for the three clades are SEQ ID NOs: 1, 2 and 3 herein. The clades are well conserved at their N- and C-termini but deviate in between. It has been found that serum raised against a given RrgB clade is active against pneumococci which express that clade, but is not active against strains which express one of the other two clades i.e. there is intra-clade cross-protection, but not inter-clade cross-protection. Thus a pneumococcal immunogen may comprise at least two different clades of RrgB. These may be present in the immunogenic composition as separate polypeptides or may be fused as a single polypeptide chain.

Thus the pneumococcal immunogen may comprise one, two or three of
(a) a first polypeptide comprising a first amino acid sequence, where the first amino acid sequence comprises an amino acid sequence (i) having at least a % sequence identity to SEQ ID NO: 1 and/or (ii) consisting of a fragment of at least x contiguous amino acids from SEQ ID NO: 1;
(b) a second polypeptide, comprising a second amino acid sequence, where the second amino acid sequence comprises an amino acid sequence (i) having at least b % sequence identity to SEQ ID NO: 2 and/or (ii) consisting of a fragment of at least y contiguous amino acids from SEQ ID NO: 2; and/or
(c) a third polypeptide, comprising a third amino acid sequence, where the third amino acid sequence comprises an amino acid sequence (i) having at least c % sequence identity to SEQ ID NO: 3 and/or (ii) consisting of a fragment of at least z contiguous amino acids from SEQ ID NO: 3.

The value of a is at least 75 e.g. 80, 85, 90, 92, 94, 95, 96, 97, 98, 99 or more. The value of b is at least 75 e.g. 80, 85, 90, 92, 94, 95, 96, 97, 98, 99 or more. The value of c is at least 75 e.g. 80, 85, 90, 92, 94, 95, 96, 97, 98, 99 or more. The values of a, b and c may be the same or different. In some embodiments, a b and c are identical. Typically, a, b and c are at least 90 e.g. at least 95.

The value of x is at least 7 e.g. 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 225, 250). The value of y is at least 7 e.g. 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 225, 250). The value of z is at least 7 e.g. 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 225, 250). The values of x, y and z may be the same or different. In some embodiments, x y and z are identical.

Fragments preferably comprise an epitope from the respective SEQ ID NO: sequence. Other useful fragments lack one or more amino acids (e.g. 1, 2, 3, 4, 5, 6, 7, 8, 9, 10, 15, 20 or more) from the C-terminus and/or one or more amino acids (e.g. 1, 2, 3, 4, 5, 6, 7, 8, 9, 10, 15, 20 or more) from the N-terminus of the respective SEQ ID NO: while retaining at least one epitope thereof. Truncation by 20-25 amino acids at the N-terminus is convenient e.g. removal of aa 1-23 of any of SEQ ID NOs: 1 to 3. A suitable fragment of SEQ ID NO: 1 is SEQ ID NO: 4. A suitable fragment of SEQ ID NO: 2 is SEQ ID NO: 5. A suitable fragment of SEQ ID NO: 3 is SEQ ID NO: 6.

The fragment of at least x contiguous amino acids from SEQ ID NO: 1 should not also be present within SEQ ID NO: 2 or within SEQ ID NO: 3. Similarly, the fragment of at least y contiguous amino acids from SEQ ID NO: 2 should not also be present within SEQ ID NO: 1 or within SEQ ID NO: 3. Similarly, the fragment of at least z contiguous amino acids from SEQ ID NO: 3 should not also be present within SEQ ID NO: 1 or within SEQ ID NO: 2. In some embodiments, therefore: a fragment of SEQ ID NO: 1 is preferably from between amino acids 31-614 of SEQ ID NO: 1; a fragment of SEQ ID NO: 2 is preferably from between amino acids 31-593 of SEQ ID NO: 2; and a fragment of SEQ ID NO: 3 is preferably from between amino acids 31-603 of SEQ ID NO: 3. In some embodiments, when a fragment from one of SEQ ID NOs: 1 to 3 is aligned as a contiguous sequence against the other two SEQ ID NOs, the identity between the fragment and each of the other two SEQ ID NOs is less than 75% e.g. less than 60%, less than 50%, less than 40%, less than 30%.

A polypeptide comprising the first amino acid sequence will, when administered to a subject, elicit an antibody response comprising antibodies that bind to the wild-type pneumococcus protein having amino acid sequence SEQ ID NO: 1 (strain TIGR4). In some embodiments these antibodies do not bind to the wild-type pneumococcus protein having amino acid sequence SEQ ID NO: 2 or to the wild-type pneumococcus protein having amino acid sequence SEQ ID NO: 3.

A polypeptide comprising the second amino acid sequence will, when administered to a subject, elicit an antibody response comprising antibodies that bind to the wild-type pneumococcus protein having amino acid sequence SEQ ID NO: 2 (strain Finland^{6B}-12). In some embodiments these antibodies do not bind to the wild-type pneumococcus protein having amino acid sequence SEQ ID NO: 1 or to the wild-type pneumococcus protein having amino acid sequence SEQ ID NO: 3.

A polypeptide comprising the third amino acid sequence will, when administered to a subject, elicit an antibody response comprising antibodies that bind to the wild-type pneumococcus protein having amino acid sequence SEQ ID NO: 3 (strain Taiwan^{23F}-15). In some embodiments these antibodies do not bind to the wild-type pneumococcus protein having amino acid sequence SEQ ID NO: 1 or to the wild-type pneumococcus protein having amino acid sequence SEQ ID NO: 2.

Although the first, second and third amino acid sequences may share some sequences in common, overall they have different amino acid sequences.

Where the invention uses only two RrgB clades a composition or polypeptide can include both: (a) a first amino acid sequence as defined above; and (b) a second amino acid sequence as defined above. In an alternative embodiment the composition includes both: (a) a first amino acid sequence as defined above; and (b) a third amino acid sequence as defined above. In an alternative embodiment the composition includes both: (a) a second amino acid sequence as defined above; and (b) a third amino acid sequence as defined above.

RrgB amino acid sequences used with the invention, may, compared to SEQ ID NOs: 1, 2 or 3, include one or more (e.g. 1, 2, 3, 4, 5, 6, 7, 8, 9, 10, etc.) conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity. The polypeptides may have one or more (e.g. 1, 2, 3, 4, 5, 6, 7, 8, 9, 10, etc.) single amino acid deletions relative to a reference sequence. The polypeptides may also include one or more (e.g. 1, 2, 3, 4, 5, 6, 7, 8, 9, 10, etc.) insertions (e.g. each of 1, 2, 3, 4 or 5 amino acids) relative to a reference sequence.

A pneumococcal immunogen used with the invention can include more than one such polypeptide. For example, the immunogen may be: (a) a mixture of spr0057, spr0096 and spr2021; (b) a mixture of spr0057, spr0565 and spr2021; (c) a mixture of spr0057, spr0096 and spr0560; (d) a mixture of spr0057, spr0096, spr0565 and spr2021; (e) a mixture of spr1418, spr0884 and spr0096; (f) a mixture of spr1418, spr0884 and spr2021; (g) a mixture of spr1418, spr0884, spr0096 and spr2021; (h) a mixture of spr0884, spr1416 and spr0057; (h) a mixture of spr0884, spr1416 and spr0096; (h) a mixture of spr0884, spr1416, spr0057 and spr0096; or (i) a mixture of spr1418, spr1431 and spr0565. Any of these mixtures (a) to (i) may also include one or more RrgB clades.

Different polypeptides (including different RrgB clades) do not have to be present as separate polypeptides but can instead be expressed as a fusion polypeptide chain. Useful fusion proteins comprise an amino acid sequence selected from the group consisting of SEQ ID NO: 11; SEQ ID NO: 13; SEQ ID NO: 15; SEQ ID NO: 17; SEQ ID NO: 19; SEQ ID NO: 21. A polypeptide comprising amino acids 1-1793 of SEQ ID NO: 15 is preferred.

### RSV Immunogens

Various RSV immunogens can be used with the invention. These will typically comprise 1, 2 or 3 of the viral F, G and M (fusion, attachment and matrix) antigens, or fragments thereof.

Reference 141 discloses subunit vaccines comprising one or more G proteins or fragments thereof, and teaches that they can be used for eliciting protective immunity without eliciting an immunopathological response.

Reference 142 discloses a vaccine based on a G protein or fragment, coupled to a support peptide. Vaccines based on G protein may be encapsulated in microspheres [143].

A useful immunogen including three F, G and M antigens is disclosed in reference 144, with best results achieved when using a composition which does not include an aluminium salt adjuvant. The F/G/M triplet of RSV antigens was also disclosed in reference 6.

Another approach uses virus-like particles (VLPs) or capsomeres which include RSV epitopes [145]. The VLPs may be based on a chimeric papillomavirus L1 polypeptide.

Live attenuated RSV vaccines are also known (e.g. see reference 146) and, if these are used with the invention, they can most usefully be combined with a live attenuated influenza vaccine (e.g. the FLUMIST™ product).

### GBS Immunogens

GBS immunogens can comprise capsular saccharides and/or on GBS proteins. Typical proteins include those disclosed in references 147 to 150. Vaccines based on conjugated capsular saccharide are discussed in reference 151. Where conjugated saccharides are included, it is preferred to include saccharides from 1 or more of GBS serotypes Ia, Ib, II, III, IV and/or V. A useful GBS immunogen may comprise a “GBS80” protein (SEQ ID NO: 67) or immunogenic fragment thereof.

### Preferred Immunogens

Preferred influenza immunogens for use with the invention are inactivated virus-derived immunogens, ideally either a split virus vaccine or purified influenza virus surface antigen vaccine. Ideally the viruses are grown on eggs or in MDCK cell culture. An influenza immunogen including a hemagglutinin from two influenza A strains (H1N1 and H3N2) and one influenza B strain is useful. The influenza immunogen may be adjuvanted e.g. with an oil-in-water emulsion adjuvant having submicron droplets.

Another preferred influenza immunogen for use with the invention is an inactivated virus-derived immunogen, ideally either a split virus vaccine or purified influenza virus surface antigen vaccine, with hemagglutinin from two influenza A strains (H1N1 and H3N2) and two influenza B strains (a B/Victoria/2/87-like influenza B virus and a B/Yamagata/16/88-like influenza B virus). The influenza immunogen may be adjuvanted e.g. with an oil-in-water emulsion adjuvant having submicron droplets. This adjuvanted 4-valent combination is particularly useful in infants ≦6 months.

A preferred pneumococcal immunogen (“Pneumo-3”) is disclosed in reference 152 and comprises the antigens “SP2216-1” (SEQ ID NO: 1 in reference 152; SEQ ID NO: 68 herein), “SP 1732-3” (SEQ ID NO: 2 in reference 152; SEQ ID NO: 69 herein) and, optionally, PsaA (SEQ ID NO: 3 in reference 152; SEQ ID NO: 70 herein). Polypeptides comprising immunogenic fragments of these SEQ ID NOs can be used in place of the actual disclosed SEQ ID NOs e.g. comprising at least one immunogenic fragment from each of SEQ ID NOs 68 & 69.

Another preferred pneumococcal immunogen comprises both spr0096 and spr2021 antigens, and in particular a fusion protein comprising both spr0096 and spr2021 e.g. comprising SEQ ID NO: 66.

Another preferred pneumococcal immunogen comprises each of the three different RrgB clades. Thus it may include (a) a first amino acid sequence comprising an amino acid sequence (i) having at least a % sequence identity to SEQ ID NO: 1 and/or (ii) consisting of a fragment of at least x contiguous amino acids from SEQ ID NO: 1; (b) a second amino acid sequence comprising an amino acid sequence (i) having at least b % sequence identity to SEQ ID NO: 2 and/or (ii) consisting of a fragment of at least y contiguous amino acids from SEQ ID NO: 2; and (c) a third amino acid sequence, comprising an amino acid sequence (i) having at least c % sequence identity to SEQ ID NO: 3 and/or (ii) consisting of a fragment of at least z contiguous amino acids from SEQ ID NO: 3. The sequences (a), (b) and (c) are ideally part of the same polypeptide chain e.g. as in SEQ ID NOs: 11, 13, 15, 17, 19 and 21 (“RrgB triple fusions”).

A possible pneumococcal immunogen (preferred if it also includes at least one pneumococcal polypeptide) is a 7-valent or 10-valent or 13-valent conjugate vaccine.

### Immunogenic Compositions

The invention provides immunogenic compositions which may be used as vaccines. These vaccines may either be prophylactic (i.e. to prevent infection) or therapeutic (i.e. to treat infection), but will typically be prophylactic.

Compositions may thus be pharmaceutically acceptable. They will usually include components in addition to the pneumococcal and influenza immunogens e.g. they typically include one or more pharmaceutical carrier(s) and/or excipient(s). A thorough discussion of such components is available in reference 228.

Compositions will generally be administered to a mammal in aqueous form. Prior to administration, however, the composition may have been in a non-aqueous form. For instance, although some vaccines are manufactured in aqueous form, then filled and distributed and administered also in aqueous form, other vaccines are lyophilised during manufacture and are reconstituted into an aqueous form at the time of use. Thus a composition of the invention may be dried, such as a lyophilised formulation.

The composition may include preservatives such as thiomersal or 2-phenoxyethanol. It is preferred, however, that the vaccine should be substantially free from (i.e. less than 5 μg/ml) mercurial material e.g. thiomersal-free. Vaccines containing no mercury are more preferred. Preservative-free vaccines are particularly preferred.

To control tonicity, it is preferred to include a physiological salt, such as a sodium salt. Sodium chloride (NaCl) is preferred, which may be present at between 1 and 20 mg/ml e.g. about 10±2 mg/ml NaCl. Other salts that may be present include potassium chloride, potassium dihydrogen phosphate, disodium phosphate dehydrate, magnesium chloride, calcium chloride, etc.

Compositions will generally have an osmolality of between 200 mOsm/kg and 400 mOsm/kg, preferably between 240-360 mOsm/kg, and will more preferably fall within the range of 290-310 mOsm/kg.

Compositions may include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range.

The pH of a composition will generally be between 5.0 and 8.1, and more typically between 6.0 and 8.0 e.g. 6.5 and 7.5, or between 7.0 and 7.8.

The composition is preferably sterile. The composition is preferably non-pyrogenic e.g. containing <1 EU (endotoxin unit, a standard measure) per dose, and preferably <0.1 EU per dose. The composition is preferably gluten free.

The composition may include material for a single immunisation, or may include material for multiple immunisations (i.e. a ‘multidose’ kit). The inclusion of a preservative is preferred in multidose arrangements. As an alternative (or in addition) to including a preservative in multidose compositions, the compositions may be contained in a container having an aseptic adaptor for removal of material.

Human vaccines are typically administered in a unit dosage volume of about 0.5 ml, although a half dose (i.e. about 0.25 ml) may be administered to children.

Immunogenic compositions of the invention may also comprise one or more immunoregulatory agents. Preferably, one or more of the immunoregulatory agents include one or more adjuvants. Adjuvants which may be used in compositions of the invention include, but are not limited to:

### A. Mineral-Containing Compositions

Mineral containing compositions suitable for use as adjuvants in the invention include mineral salts, such as aluminium salts and calcium salts. The invention includes mineral salts such as hydroxides (e.g. oxyhydroxides), phosphates (e.g. hydroxyphosphates, orthophosphates), sulphates, etc. [e.g. see chapters 8 & 9 of ref. 156], or mixtures of different mineral compounds, with the compounds taking any suitable form (e.g. gel, crystalline, amorphous, etc.), and with adsorption being preferred. The mineral containing compositions may also be formulated as a particle of metal salt.

The adjuvants known as “aluminium hydroxide” are typically aluminium oxyhydroxide salts, which are usually at least partially crystalline. Aluminium oxyhydroxide, which can be represented by the formula AlO(OH), can be distinguished from other aluminium compounds, such as aluminium hydroxide Al(OH)₃, by infrared (IR) spectroscopy, in particular by the presence of an adsorption band at 1070 cm⁻¹ and a strong shoulder at 3090-3100 cm⁻¹ [chapter 9 of ref. 156]. The degree of crystallinity of an aluminium hydroxide adjuvant is reflected by the width of the diffraction band at half height (WHH), with poorly-crystalline particles showing greater line broadening due to smaller crystallite sizes. The surface area increases as WHH increases, and adjuvants with higher WHH values have been seen to have greater capacity for antigen adsorption. A fibrous morphology (e.g. as seen in transmission electron micrographs) is typical for aluminium hydroxide adjuvants. The pI of aluminium hydroxide adjuvants is typically about 11 i.e. the adjuvant itself has a positive surface charge at physiological pH. Adsorptive capacities of between 1.8-2.6 mg protein per mg Al⁺⁺⁺ at pH 7.4 have been reported for aluminium hydroxide adjuvants.

The adjuvants known as “aluminium phosphate” are typically aluminium hydroxyphosphates, often also containing a small amount of sulfate (i.e. aluminium hydroxyphosphate sulfate). They may be obtained by precipitation, and the reaction conditions and concentrations during precipitation influence the degree of substitution of phosphate for hydroxyl in the salt. Hydroxyphosphates generally have a PO₄/Al molar ratio between 0.3 and 1.2. Hydroxyphosphates can be distinguished from strict AlPO₄ by the presence of hydroxyl groups. For example, an IR spectrum band at 3164 cm⁻¹ (e.g. at 200° C.) indicates the presence of structural hydroxyls [ch. 9 of ref 156].

The PO₄/Al³⁺ molar ratio of an aluminium phosphate adjuvant will generally be between 0.3 and 1.2, preferably between 0.8 and 1.2, and more preferably 0.95±0.1. The aluminium phosphate will generally be amorphous, particularly for hydroxyphosphate salts. A typical adjuvant is amorphous aluminium hydroxyphosphate with PO₄/Al molar ratio between 0.84 and 0.92, included at 0.6 mg Al³⁺/ml. The aluminium phosphate will generally be particulate (e.g. plate-like morphology as seen in transmission electron micrographs). Typical diameters of the particles are in the range 0.5-20 μm (e.g. about 5-10 μm) after any antigen adsorption. Adsorptive capacities of between 0.7-1.5 mg protein per mg Al⁺⁺⁺ at pH 7.4 have been reported for aluminium phosphate adjuvants.

The point of zero charge (PZC) of aluminium phosphate is inversely related to the degree of substitution of phosphate for hydroxyl, and this degree of substitution can vary depending on reaction conditions and concentration of reactants used for preparing the salt by precipitation. PZC is also altered by changing the concentration of free phosphate ions in solution (more phosphate=more acidic PZC) or by adding a buffer such as a histidine buffer (makes PZC more basic). Aluminium phosphates used according to the invention will generally have a PZC of between 4.0 and 7.0, more preferably between 5.0 and 6.5 e.g. about 5.7.

Suspensions of aluminium salts used to prepare compositions of the invention may contain a buffer (e.g. a phosphate or a histidine or a Tris buffer), but this is not always necessary. The suspensions are preferably sterile and pyrogen-free. A suspension may include free aqueous phosphate ions e.g. present at a concentration between 1.0 and 20 mM, preferably between 5 and 15 mM, and more preferably about 10 mM. The suspensions may also comprise sodium chloride.

In one embodiment, an adjuvant component includes a mixture of both an aluminium hydroxide and an aluminium phosphate. In this case there may be more aluminium phosphate than hydroxide e.g. a weight ratio of at least 2:1 e.g. ≧5:1, ≧6:1, ≧7:1, ≧8:1, ≧9:1, etc.

The known PREVNAR™ and SYNFLORIX™ vaccines both include an aluminium phosphate adjuvant. This adjuvant is not ideal for use with influenza vaccines, and may also be more suitable for pneumococcal saccharide antigens than for protein antigens. Thus, in some embodiments where a composition includes both an influenza virus immunogen and a pneumococcal protein immunogen, a composition may be free from an aluminium phosphate adjuvant. If an aluminium phosphate adjuvant is present, though, it may be in combination with a second adjuvant e.g. 3dMPL or an oil-in-water emulsion. The inclusion of an aluminium phosphate salts as the sole adjuvant can thus be avoided.

The concentration of Al⁺⁺⁺ in a composition for administration to a patient is preferably less than 10 mg/ml e.g. ≦5 mg/ml, ≦4 mg/ml, ≦3 mg/ml, ≦2 mg/ml, ≦1 mg/ml, etc. A preferred range is between 0.3 and 1 mg/ml. A maximum of <0.85 mg/dose is preferred.

### B. Oil Emulsions

Oil emulsion compositions suitable for use as adjuvants in the invention include squalene-water emulsions, such as MF59 [Chapter 10 of ref 156; see also ref. 153] (5% Squalene, 0.5% Tween 80, and 0.5% Span 85, formulated into submicron particles using a microfluidizer). Complete Freund's adjuvant (CFA) and incomplete Freund's adjuvant (IFA) may also be used.

Various suitable oil-in-water emulsions are known, and they typically include at least one oil and at least one surfactant, with the oil(s) and surfactant(s) being biodegradable (metabolisable) and biocompatible. The oil droplets in the emulsion are generally less than 5 μm in diameter, and advantageously the emulsion comprises oil droplets with a sub-micron diameter, with these small sizes being achieved with a microfluidiser to provide stable emulsions. Droplets with a size less than 220 nm are preferred as they can be subjected to filter sterilization.

The invention can be used with oils such as those from an animal (such as fish) or vegetable source. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and may therefore be used in the practice of this invention. The procedures for separation, purification, saponification and other means necessary for obtaining pure oils from animal sources are well known in the art. Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Shark liver oil contains a branched, unsaturated terpenoid known as squalene, 2,6,10,15,19,23-hexamethyl-2,6,10,14,18,22-tetracosahexaene. Other preferred oils are the tocopherols (see below). Oil in water emulsions comprising sqlauene are particularly preferred. Mixtures of oils can be used.

Surfactants can be classified by their ‘HLB’ (hydrophile/lipophile balance). Preferred surfactants of the invention have a HLB of at least 10, preferably at least 15, and more preferably at least 16. The invention can be used with surfactants including, but not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Preferred surfactants for including in the emulsion are Tween 80 (polyoxyethylene sorbitan monooleate), Span 85 (sorbitan trioleate), lecithin and Triton X-100. As mentioned above, detergents such as Tween 80 may contribute to the thermal stability seen in the examples below.

Mixtures of surfactants can be used e.g. Tween 80/Span 85 mixtures. A combination of a polyoxyethylene sorbitan ester such as polyoxyethylene sorbitan monooleate (Tween 80) and an octoxynol such as t-octylphenoxypolyethoxyethanol (Triton X-100) is also suitable. Another useful combination comprises laureth 9 plus a polyoxyethylene sorbitan ester and/or an octoxynol.

Preferred amounts of surfactants (% by weight) are: polyoxyethylene sorbitan esters (such as Tween 80) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, preferably 0.1 to 10% and in particular 0.1 to 1% or about 0.5%.

Specific oil-in-water emulsion adjuvants useful with the invention include, but are not limited to:
A submicron emulsion of squalene, Tween 80, and Span 85. The composition of the emulsion by volume can be about 5% squalene, about 0.5% polysorbate 80 and about 0.5% Span 85. In weight terms, these ratios become 4.3% squalene, 0.5% polysorbate 80 and 0.48% Span 85. This adjuvant is known as ‘MF59’ [153-155], as described in more detail in Chapter 10 of ref. 156 and chapter 12 of ref. 157. The MF59 emulsion advantageously includes citrate ions e.g. 10 mM sodium citrate buffer.
An emulsion comprising squalene, an α-tocopherol, and polysorbate 80. These emulsions may have from 2 to 10% squalene, from 2 to 10% tocopherol and from 0.3 to 3% Tween 80, and the weight ratio of squalene:tocopherol is preferably ≦1 (e.g. 0.90) as this provides a more stable emulsion. Squalene and Tween 80 may be present volume ratio of about 5:2, or at a weight ratio of about 11:5. One such emulsion can be made by dissolving Tween 80 in PBS to give a 2% solution, then mixing 90 ml of this solution with a mixture of (5 g of DL-α-tocopherol and 5 ml squalene), then microfluidising the mixture. The resulting emulsion may have submicron oil droplets e.g. with an average diameter of between 100 and 250 nm, preferably about 180 nm.
An emulsion of squalene, a tocopherol, and a Triton detergent (e.g. Triton X-100). The emulsion may also include a 3d-MPL (see below). The emulsion may contain a phosphate buffer.
An emulsion comprising a polysorbate (e.g. polysorbate 80), a Triton detergent (e.g. Triton X-100) and a tocopherol (e.g. an α-tocopherol succinate). The emulsion may include these three components at a mass ratio of about 75:11:10 (e.g. 750 μg/ml polysorbate 80, 110 μg/ml Triton X-100 and 100 μg/ml α-tocopherol succinate), and these concentrations should include any contribution of these components from antigens. The emulsion may also include squalene. The emulsion may also include a 3d-MPL (see below). The aqueous phase may contain a phosphate buffer.
An emulsion of squalane, polysorbate 80 and poloxamer 401 (“Pluronic™ L121”). The emulsion can be formulated in phosphate buffered saline, pH 7.4. This emulsion is a useful delivery vehicle for muramyl dipeptides, and has been used with threonyl-MDP in the “SAF-1” adjuvant [158] (0.05-1% Thr-MDP, 5% squalane, 2.5% Pluronic L121 and 0.2% polysorbate 80). It can also be used without the Thr-MDP, as in the “AF” adjuvant [159] (5% squalane, 1.25% Pluronic L121 and 0.2% polysorbate 80). Microfluidisation is preferred.
An emulsion comprising squalene, an aqueous solvent, a polyoxyethylene alkyl ether hydrophilic nonionic surfactant (e.g. polyoxyethylene (12) cetostearyl ether) and a hydrophobic nonionic surfactant (e.g. a sorbitan ester or mannide ester, such as sorbitan monoleate or ‘Span 80’). The emulsion is preferably thermoreversible and/or has at least 90% of the oil droplets (by volume) with a size less than 200 nm [160]. The emulsion may also include one or more of alditol; a cryoprotective agent (e.g. a sugar, such as dodecylmaltoside and/or sucrose); and/or an alkylpolyglycoside. Such emulsions may be lyophilized.
An emulsion having from 0.5-50% of an oil, 0.1-10% of a phospholipid, and 0.05-5% of a non-ionic surfactant. As described in reference 161, preferred phospholipid components are phosphatidylcholine, phosphatidylethanolamine, phosphatidylserine, phosphatidylinositol, phosphatidylglycerol, phosphatidic acid, sphingomyelin and cardiolipin. Submicron droplet sizes are advantageous.
A submicron oil-in-water emulsion of a non-metabolisable oil (such as light mineral oil) and at least one surfactant (such as lecithin, Tween 80 or Span 80). Additives may be included, such as QuilA saponin, cholesterol, a saponin-lipophile conjugate (such as GPI-0100, described in reference 162, produced by addition of aliphatic amine to desacylsaponin via the carboxyl group of glucuronic acid), dimethyldioctadecylammonium bromide and/or N,N-dioctadecyl-N,N-bis(2-hydroxyethyl)propanediamine.
An emulsion comprising a mineral oil, a non-ionic lipophilic ethoxylated fatty alcohol, and a non-ionic hydrophilic surfactant (e.g. an ethoxylated fatty alcohol and/or polyoxyethylene-polyoxypropylene block copolymer) [163].
An emulsion comprising a mineral oil, a non-ionic hydrophilic ethoxylated fatty alcohol, and a non-ionic lipophilic surfactant (e.g. an ethoxylated fatty alcohol and/or polyoxyethylene-polyoxypropylene block copolymer) [163].
An emulsion in which a saponin (e.g. QuilA or QS21) and a sterol (e.g. a cholesterol) are associated as helical micelles [164].

The use of oil-in-water emulsions as adjuvants with the invention is particularly useful in children. These adjuvants can provide high and sustained antibody titers against influenza viruses for at least 6 months, and the elicited immune responses are cross-reactive against drift variants of circulating influenza virus strains [165]. Infants under 6 months currently have the highest influenza hospitalization rate of any age group, hence there is a need for effective prevention in this age group.

Antigens and adjuvants in a composition will typically be in admixture at the time of delivery to a patient. The emulsions may be mixed with antigen during manufacture, or extemporaneously, at the time of delivery. Thus the adjuvant and antigen may be kept separately in a packaged or distributed vaccine, ready for final formulation at the time of use. The antigen will generally be in an aqueous form, such that the vaccine is finally prepared by mixing two liquids. The volume ratio of the two liquids for mixing can vary (e.g. between 5:1 and 1:5) but is generally about 1:1.

### C. Saponin Formulations [Chapter 22 of Ref. 156]

Saponin formulations may also be used as adjuvants in the invention. Saponins are a heterogeneous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs. QS21 is marketed as Stimulon™

Saponin compositions have been purified using HPLC and RP-HPLC. Specific purified fractions using these techniques have been identified, including QS7, QS17, QS18, QS21, QH-A, QH-B and QH-C. Preferably, the saponin is QS21. A method of production of QS21 is disclosed in ref. 166. Saponin formulations may also comprise a sterol, such as cholesterol [167].

Combinations of saponins and cholesterols can be used to form unique particles called immunostimulating complexs (ISCOMs) [chapter 23 of ref. 156]. ISCOMs typically also include a phospholipid such as phosphatidylethanolamine or phosphatidylcholine. Any known saponin can be used in ISCOMs. Preferably, the ISCOM includes one or more of QuilA, QHA & QHC. ISCOMs are further described in refs. 167-169. Optionally, the ISCOMS may be devoid of additional detergent [170].

A review of the development of saponin based adjuvants can be found in refs. 171 & 172.

### D. Bacterial or Microbial Derivatives

Adjuvants suitable for use in the invention include bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof.

Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. A preferred “small particle” form of 3 De-O-acylated monophosphoryl lipid A is disclosed in ref. 173. Such “small particles” of 3dMPL are small enough to be sterile filtered through a 0.24 μm membrane [173]. Other non-toxic LPS derivatives include monophosphoryl lipid A mimics, such as aminoalkyl glucosaminide phosphate derivatives e.g. RC-529 [174,175].

Lipid A derivatives include derivatives of lipid A from Escherichia coli such as OM-174. OM-174 is described for example in refs. 176 & 177.

Immunostimulatory oligonucleotides suitable for use as adjuvants in the invention include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory.

The CpG's can include nucleotide modifications/analogs such as phosphorothioate modifications and can be double-stranded or single-stranded. References 178, 179 and 180 disclose possible analog substitutions e.g. replacement of guanosine with 2′-deoxy-7-deazaguanosine. The adjuvant effect of CpG oligonucleotides is further discussed in refs. 181-186.

The CpG sequence may be directed to TLR9, such as the motif GTCGTT or TTCGTT [187]. The CpG sequence may be specific for inducing a Th1 immune response, such as a CpG-A ODN, or it may be more specific for inducing a B cell response, such a CpG-B ODN. CpG-A and CpG-B ODNs are discussed in refs. 188-190. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, refs. 187 & 191-193.

A particularly useful adjuvant based around immunostimulatory oligonucleotides is known as IC-31™ [194-196]. Thus an adjuvant used with the invention may comprise a mixture of (i) an oligonucleotide (e.g. between 15-40 nucleotides) including at least one (and preferably multiple) CpI motifs (i.e. a cytosine linked to an inosine to form a dinucleotide), and (ii) a polycationic polymer, such as an oligopeptide (e.g. between 5-20 amino acids) including at least one (and preferably multiple) Lys-Arg-Lys tripeptide sequence(s). The oligonucleotide may be a deoxynucleotide comprising 26-mer sequence 5′-(IC)₁₃-3′ (SEQ ID NO: 71). The polycationic polymer may be a peptide comprising 11-mer amino acid sequence KLKLLLLLKLK (SEQ ID NO: 72). This combination of SEQ ID NOs: 71 and 72 provides the IC-31™ adjuvant.

Bacterial ADP-ribosylating toxins and detoxified derivatives thereof may be used as adjuvants in the invention. Preferably, the protein is derived from E. coli (E. coli heat labile enterotoxin “LT”), cholera (“CT”), or pertussis (“PT”). The use of detoxified ADP-ribosylating toxins as mucosal adjuvants is described in ref. 197 and as parenteral adjuvants in ref. 198. The toxin or toxoid is preferably in the form of a holotoxin, comprising both A and B subunits. Preferably, the A subunit contains a detoxifying mutation; preferably the B subunit is not mutated. Preferably, the adjuvant is a detoxified LT mutant such as LT-K63, LT-R72, and LT-G192. The use of ADP-ribosylating toxins and detoxified derivatives thereof, particularly LT-K63 and LT-R72, as adjuvants can be found in refs. 199-206. A useful CT mutant is or CT-E29H [207]. Numerical reference for amino acid substitutions is preferably based on the alignments of the A and B subunits of ADP-ribosylating toxins set forth in ref. 208, specifically incorporated herein by reference in its entirety.

### E. Human Immunomodulators

Human immunomodulators suitable for use as adjuvants in the invention include cytokines, such as interleukins (e.g. IL-1, IL-2, IL-4, IL-5, IL-6, IL-7, IL-12 [209], etc.) [210], interferons (e.g. interferon-γ), macrophage colony stimulating factor, and tumor necrosis factor. A preferred immunomodulator is IL-12.

### F. Bioadhesives and Mucoadhesives

Bioadhesives and mucoadhesives may also be used as adjuvants in the invention. Suitable bioadhesives include esterified hyaluronic acid microspheres [211] or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention [212].

### G. Microparticles

Microparticles may also be used as adjuvants in the invention. Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

### H. Liposomes (Chapters 13 & 14 of Ref 156)

Examples of liposome formulations suitable for use as adjuvants are described in refs. 213-215.

### I. Imidazoquinolone Compounds.

Examples of imidazoquinolone compounds suitable for use adjuvants in the invention include Imiquamod and its homologues (e.g. “Resiquimod 3M”), described further in refs. 216 and 217.

The invention may also comprise combinations of aspects of one or more of the adjuvants identified above. For example, the following adjuvant compositions may be used in the invention: (1) a saponin and an oil-in-water emulsion [218]; (2) a saponin (e.g. QS21)+a non-toxic LPS derivative (e.g. 3dMPL) [219]; (3) a saponin (e.g. QS21)+a non-toxic LPS derivative (e.g. 3dMPL)+a cholesterol; (4) a saponin (e.g. QS21)+3dMPL+IL-12 (optionally+a sterol) [220]; (5) combinations of 3dMPL with, for example, QS21 and/or oil-in-water emulsions [221]; (6) SAF, containing 10% squalane, 0.4% Tween 80™, 5% pluronic-block polymer L121, and thr-MDP, either microfluidized into a submicron emulsion or vortexed to generate a larger particle size emulsion. (7) Ribi™ adjuvant system (RAS), (Ribi Immunochem) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); and (8) one or more mineral salts (such as an aluminum salt)+a non-toxic derivative of LPS (such as 3dMPL).

Other substances that act as immunostimulating agents are disclosed in chapter 7 of ref. 156.

An aluminium hydroxide adjuvant is useful, and antigens are generally adsorbed to this salt. Oil-in-water emulsions comprising squalene, with submicron oil droplets, are also preferred, particularly in the elderly. Useful adjuvant combinations include combinations of Th1 and Th2 adjuvants such as CpG & an aluminium salt, or resiquimod & an aluminium salt. A combination of an aluminium salt and 3dMPL may be used.

Immunogenic compositions used as vaccines comprise an immunologically effective amount of the pneumococcal and influenza immunogens, as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials. Dosing guidance is already available from the authorised human pneumococcal and influenza vaccines.

Pneumococcal and influenza infections can affect various areas of the body and so the compositions of the invention may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition or a spray-freeze dried composition). The composition may be prepared for topical administration e.g. as an ointment, cream or powder. The composition may be prepared for oral administration e.g. as a tablet or capsule, as a spray, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops. Usually, though, the composition will be an injectable liquid, suitable for intramuscular injection, which is the current administration route for both inactivated influenza vaccines and pneumococcal vaccines, and usually with a unit dosage volume of 0.5 ml.

Because influenza vaccines are prepared on a seasonal basis, but pneumococcal vaccines are not, it may be convenient to distribute kits with components which can be combined at the point of use to provide the immunogenic compositions of the invention. This arrangement permits, for example, a pneumococcal or RSV vaccine to be preserved between influenza seasons. Thus the invention provides a kit comprising (i) a first kit component comprising an influenza virus immunogen and (ii) a second kit component comprising a pneumococcal immunogen. Mixing the two kit components provides a composition of the invention. The second kit component can be in dried form, in which case it can be reconstituted by an influenza virus immunogen to provide the composition of the invention. If the first and second components are both in liquid form, their immunogens should be more concentrated than the desired final concentration, such that their mixing provides mutual dilution to the final dosage concentration. For example, the two liquid immunogens can be provided at double concentration, such that a 1:1 (volume) mixing provides the required final concentration.

Where such a kit is provided, it may comprise two vials, or it may comprise one ready-filled syringe and one vial, with the contents of the syringe being used to reactivate the contents of the vial prior to injection. Other arrangements are also possible.

Where the two immunogens are presented in kit form, one or both may include an adjuvant. In other embodiments, however, the kit includes a third kit component comprising an adjuvant, in which case the third component can be combined with unadjuvanted first and second components to provide a final adjuvanted composition. In one useful kit the influenza immunogen is adjuvanted (e.g. with an oil-in-water emulsion adjuvant) whereas the pneumococcal immunogen is unadjuvanted, such that their mixing provides an adjuvanted composition of the invention. In another useful kit the influenza immunogen is unadjuvanted whereas the pneumococcal immunogen is adjuvanted, such that their mixing provides an adjuvanted composition of the invention. In another useful kit the influenza immunogen and pneumococcal immunogen are both adjuvanted, but with different adjuvants.

### Methods of Treatment, and Administration of the Vaccine

The invention also provides a method for raising an immune response in a mammal comprising the step of administering an effective amount of an immunogenic composition of the invention. The immune response is preferably protective and preferably involves antibodies and/or cell-mediated immunity. The method may raise a booster response.

The invention also provides an influenza virus immunogen and a pneumococcal immunogen for use as a combined medicament e.g. for use in raising an immune response in a mammal. The pneumococcal immunogen will typically comprise at least one pneumococcal polypeptide. The medicament may also include a RSV immunogen, but in some embodiments the medicament does not include a RSV immunogen.

The invention also provides (i) a pneumococcal immunogen comprising at least one pneumococcal polypeptide and (ii) an influenza virus immunogen and/or a RSV immunogen, for use as a combined medicament e.g. for use in raising an immune response in a mammal.

The invention also provides the use of an influenza virus immunogen and a pneumococcal immunogen in the manufacture of a combined medicament for raising an immune response in a mammal. The pneumococcal immunogen will typically comprise at least one pneumococcal polypeptide. The medicament may also include a RSV immunogen, but in some embodiments the medicament does not include a RSV immunogen.

The invention also provides the use of (i) a pneumococcal immunogen comprising at least one pneumococcal polypeptide and (ii) an influenza virus immunogen and/or a RSV immunogen, in the manufacture of a combined medicament for raising an immune response in a mammal.

By raising an immune response in the mammal by these uses and methods, the mammal can be protected both against pneumococcus and influenza. Thus the composition may be used for active immunisation against (a) invasive disease (e.g. including bacteremia, sepsis, meningitis, bacteremic pneumonia, and/or acute otitis media) caused by S. pneumoniae and (b) influenza virus disease and/or infection, in particular caused by influenza virus types A and B. In combination, therefore, the combination can be effective against multiple lower respiratory tract diseases.

The invention also provides a delivery device pre-filled with an immunogenic composition of the invention. Suitable delivery devices include pre-filled syringes.

The mammal is preferably a human. Where the vaccine is for prophylactic use, the human is preferably a child (e.g. a toddler or infant) or a teenager; where the vaccine is for therapeutic use, the human is preferably a teenager or an adult. A vaccine intended for children may also be administered to adults e.g. to assess safety, dosage, immunogenicity, etc. Vaccines prepared according to the invention may be used to treat both children and adults. Thus a human patient may be less than 1 year old, less than 5 years old, 1-5 years old, 5-15 years old, 15-55 years old, or at least 55 years old. Preferred patients for receiving the vaccines are the elderly (e.g. ≧50 years old, ≧60 years old, and preferably ≧65 years). The vaccines are not suitable solely for these age groups, however, and may be used more generally in a population, including for the young (e.g. ≦5 years old), hospitalised patients, healthcare workers, armed service and military personnel, pregnant women, the chronically ill, or immunodeficient patients.

One way of checking efficacy of therapeutic treatment involves monitoring pneumococcal or influenza infection after administration of the compositions of the invention. One way of checking efficacy of prophylactic treatment involves testing post-immunisation sera in standard tests. For example, to check anti-pneumococcal immunity sera can be tested in an opsonophagocytic killing assay (OPKA), with the ability to opsonise pneumococcal bacteria indicating protective efficacy. Another way of checking efficacy of prophylactic anti-pneumococcal treatment involves post-immunisation challenge in an animal model of pneumococcal infection, e.g., guinea pigs or mice. One such model is described in reference 222. To check anti-influenza immunity, standard in vitro tests can be used such as testing hemagglutination titers or microneutralisation titers. Preferred compositions of the invention will satisfy 1, 2 or 3 of the CPMP criteria for adult efficacy for each influenza strain, even though they are administered to children. These criteria are: (1) ≧70% seroprotection; (2) ≧40% seroconversion or significant increase; and/or (3) a GMT increase of ≧2.5-fold. In elderly (>60 years), these criteria are: (1) ≧60% seroprotection; (2) ≧30% seroconversion; and/or (3) a GMT increase of ≧2-fold. These CPMP criteria are based on open label studies with at least 50 patients.

Compositions of the invention may be suitable for reducing medically-attended febrile illness, acute otitis media, and/or lower-respiratory infections (including pneumonia).

Compositions of the invention will generally be administered directly to a patient. Direct delivery may be accomplished by parenteral injection (e.g. subcutaneously, intraperitoneally, intravenously, intramuscularly, or to the interstitial space of a tissue), or mucosally, such as by rectal, oral (e.g. tablet, spray), vaginal, topical, transdermal or transcutaneous, intranasal, ocular, aural, pulmonary or other mucosal administration. Intramuscular administration is typical, as discussed above.

The invention may be used to elicit systemic and/or mucosal immunity, preferably to elicit an enhanced systemic and/or mucosal immunity.

Dosage can be by a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Multiple doses will typically be administered at least 1 week apart (e.g. about 2 weeks, about 3 weeks, about 4 weeks, about 6 weeks, about 8 weeks, about 10 weeks, about 12 weeks, about 16 weeks, etc.). Immunogenic compositions of the invention can be administered to the same patient every year, every 2 years, every 3 years, etc.

One way of using the compositions of the invention (e.g. in children <15 years old, or elderly >55 years old) is to administer a combined vaccine as defined herein (e.g. with pneumococcal and influenza immunogens) and then to give non-combined influenza vaccines (e.g. a normal trivalent seasonal influenza vaccine) in subsequent seasons. The invention provides a method for immunising a patient, comprising (i) administering an immunogenic composition of the invention, wherein the composition includes an influenza virus immunogens, then, at least 3 months later, (ii) administering an immunogenic composition in which influenza virus immunogens are the sole immunogenic component. The invention also provides a method for immunising a patient, comprising administering to a patient an immunogenic composition in which influenza virus immunogens are the sole immunogenic component, wherein the patient has previously been immunised with an immunogenic composition of the invention which includes an influenza virus immunogens.

Vaccines produced by the invention may be administered to patients at substantially the same time as (e.g. during the same medical consultation or visit to a healthcare professional or vaccination centre) other vaccines e.g. at substantially the same time as a measles vaccine, a mumps vaccine, a rubella vaccine, a MMR vaccine, a varicella vaccine, a MMRV vaccine, a diphtheria vaccine, a tetanus vaccine, a pertussis vaccine, a DTP vaccine, a conjugated H. influenzae type b vaccine, an inactivated poliovirus vaccine, a hepatitis B virus vaccine, a meningococcal conjugate vaccine (such as a tetravalent A-C-W135-Y vaccine), a respiratory syncytial virus vaccine, etc.

One way of using the compositions of the invention (e.g. in children <1 year old) is to administer three doses spaced 2 months apart e.g. at 2, 4 and 6 months of age. These immunisations can be given at the same time as other pediatric vaccines e.g. at the same time as a DTP-containing vaccine (such as a DTaP-containing vaccine). Thus, unlike typical usage of trivalent influenza vaccines in children, compositions of the invention can be used in an age-based schedule rather than in a seasonal schedule. This administration schedule is particularly useful with a vaccine comprising a pneumococcal polypeptide, hemagglutinin from each of a H1N1 influenza A virus, a H3N2 influenza A virus, a B/Victoria/2/87-like influenza B virus and a B/Yamagata/16/88-like influenza B virus, and an oil-in-water emulsion adjuvant.

### Polypeptides

Polypeptides used with the invention (e.g. as part of the pneumococcal immunogen) can be prepared in many ways e.g. by chemical synthesis (in whole or in part), by digesting longer polypeptides using proteases, by translation from RNA, by purification from cell culture (e.g. from recombinant expression), from the organism itself (e.g. after bacterial culture, or direct from patients), etc. A preferred method for production of peptides <40 amino acids long involves in vitro chemical synthesis [223,224]. Solid-phase peptide synthesis is particularly preferred, such as methods based on tBoc or Fmoc [225] chemistry. Enzymatic synthesis [226] may also be used in part or in full. As an alternative to chemical synthesis, biological synthesis may be used e.g. the polypeptides may be produced by translation. This may be carried out in vitro or in vivo. Biological methods are in general restricted to the production of polypeptides based on L-amino acids, but manipulation of translation machinery (e.g. of aminoacyl tRNA molecules) can be used to allow the introduction of D-amino acids (or of other non natural amino acids, such as iodotyrosine or methylphenylalanine, azidohomoalanine, etc.) [227]. Where D-amino acids are included, however, it is preferred to use chemical synthesis. Polypeptides may have covalent modifications at the C-terminus and/or N-terminus.

Polypeptides can take various forms (e.g. native, glycosylated, non-glycosylated, lipidated, non-lipidated, phosphorylated, non-phosphorylated, myristoylated, non-myristoylated, monomeric, multimeric, particulate, denatured, etc.).

Polypeptides are preferably provided in purified or substantially purified form i.e. substantially free from other polypeptides (e.g. free from naturally-occurring polypeptides), particularly from other pneumococcal or host cell polypeptides, and are generally at least about 50% pure (by weight), and usually at least about 90% pure i.e. less than about 50%, and more preferably less than about 10% (e.g. 5% or less) of a composition is made up of other expressed polypeptides.

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can occur as single chains or associated chains. Polypeptides can be naturally or non-naturally glycosylated (i.e. the polypeptide has a glycosylation pattern that differs from the glycosylation pattern found in the corresponding naturally occurring polypeptide).

Although expression of the polypeptide may take place in a Streptococcus, the invention will usually use a heterologous host for recombinant expression. The heterologous host may be prokaryotic (e.g. a bacterium) or eukaryotic. It will usually be E. coli, but other suitable hosts include Bacillus subtilis, Vibrio cholerae, Salmonella typhi, Salmonella typhimurium, Neisseria lactamica, Neisseria cinerea, Mycobacteria (e.g. M. tuberculosis), yeasts, etc.

### General

The practice of the present invention will employ, unless otherwise indicated, conventional methods of chemistry, biochemistry, molecular biology, immunology and pharmacology, within the skill of the art. Such techniques are explained fully in the literature. See, e.g., references 228-235, etc.

“GI” numbering is used above. A GI number, or “GenInfo Identifier”, is a series of digits assigned consecutively to each sequence record processed by NCBI when sequences are added to its databases. The GI number bears no resemblance to the accession number of the sequence record. When a sequence is updated (e.g. for correction, or to add more annotation or information) then it receives a new GI number. Thus the sequence associated with a given GI number is never changed.

Where the invention concerns an “epitope”, this epitope may be a B-cell epitope and/or a T-cell epitope. Such epitopes can be identified empirically (e.g. using PEPSCAN [236,237] or similar methods), or they can be predicted (e.g. using the Jameson-Wolf antigenic index [238], matrix-based approaches [239], MAPITOPE [240], TEPITOPE [241,242], neural networks [243], OptiMer & EpiMer [244, 245], ADEPT [246], Tsites [247], hydrophilicity [248], antigenic index [249] or the methods disclosed in references 250-254, etc.). Epitopes are the parts of an antigen that are recognised by and bind to the antigen binding sites of antibodies or T-cell receptors, and they may also be referred to as “antigenic determinants”.

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x+10%.

Unless specifically stated, a process comprising a step of mixing two or more components does not require any specific order of mixing. Thus components can be mixed in any order. Where there are three components then two components can be combined with each other, and then the combination may be combined with the third component, etc.

Antibodies will generally be specific for their target. Thus they will have a higher affinity for the target than for an irrelevant control protein, such as bovine serum albumin.

References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of ref. 255. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in ref. 256.

### BRIEF DESCRIPTION OF THE DRAWINGS

FIG. 1 shows 50% neutralisation titers using sera from 11 test groups of mice. Each group has two bars of data, representing MN titers against A/H1N1 (left) and A/H3N2 (right). The 11 groups, from left to right, are: RrgB-321+MF59; RrgB-213+MF59; influenza alone; influenza+MF59; RrgB-321+influenza/MF59; RrgB-213 +influenza/MF59; MF59 alone; influenza+aluminium hydroxide; RrgB-321+influenza/aluminium hydroxide; RrgB-213+influenza/aluminium hydroxide; buffer.

FIG. 2 shows HI titers (GMT, log-2 scale) for the same 11 test groups as FIG. 1. Each group has three bars of data, representing MN titers against A/H1N1 (left), A/H3N2 (middle) or B (right).

FIG. 3 shows OPKA results (% killing) using the indicated dilution of sera. The lines show data for six groups and, from top to bottom for the 1/12 dilution, these are: ∘ Prevnar control; Δ anti-6B control; ⋄ RrgB-321+MF59; Δ RrgB-321+influenza+MF59; □ RrgB-321+influenza+aluminium hydroxide; and Δinfluenza alone.

### MODES FOR CARRYING OUT THE INVENTION

### Preliminary Experiments

6 weeks old BalB/c mice, 8 mice per group, are immunised at days 0, 14 and 28. Compositions are administered intramuscularly. Mice are then challenged intranasally with the TIGR4 strain of pneumococcus and are assessed for in vivo protection (mortality) and in vitro protection (opsonophagocytic killing assay). Blood is taken from the mice before the challenge and assessed for influenza seroconversion.

A first experiment uses 11 groups of mice who receive a pneumococcal immunogen (either 20 μg of a “RrgB triple fusion” protein, and/or 150 μg of the ‘Pneumo-3’ combination at 50 μg per polypeptide), an influenza immunogen (the Agrippal™ or Fluad™ products at 0.1 μg/strain), or a mixture of the two. The compositions are adjuvanted with aluminium hydroxide and a further control group receives the adjuvant alone. The 11 groups receive immunogens as follows:
1. RrgB triple fusion+adjuvant
2. Pneumo-3+adjuvant
3. Agrippal™+adjuvant
4. RrgB triple fusion+Pneumo-3+Agrippal™+adjuvant
5. RrgB triple fusion+Pneumo-3+adjuvant
6. Pneumo-3+Agrippal™+adjuvant
7. RrgB+Agrippal™+adjuvant
8. Adjuvant
9. Agrippal™
10. Fluad™
11. RrgB triple fusion+Pneumo-3+Fluad™

A second experiment uses 8 groups of mice who receive a pneumococcal immunogen, an influenza immunogen, or a mixture of the two. The compositions are adjuvanted with MF59. The 8 groups are:
1. RrgB triple fusion+MF59
2. Pneumo-3+MF59
3. Fluad™
4. RrgB triple fusion+Pneumo-3+Fluad™+MF59
5. RrgB triple fusion+Pneumo-3+MF59
6. Pneumo-3+Fluad™+MF59
7. RrgB triple fusion+Fluad™+MF59
8. MF59
   Functional Immunology Assays with Combination Vaccines

Two different RrgB triple fusions, referred to as ‘213’ (SEQ ID NO: 21) or ‘321’ (SEQ ID NO: 15) are combined with trivalent seasonal influenza vaccine, either unadjuvanted or adjuvanted with either MF59 or aluminium hydroxide. These combinations are used to immunise mice.

Mice are immunized intramuscularly with different combinations of the RrgB triple fusion and influenza vaccine. Sera from immunised mice are evaluated by influenza hemagglutination inhibition (HI) and microneutralization (MN) assays, and also in an opsonophagocytosis killing assay (OPKA). There are 11 groups in total. At day 0 mice receive one of the RrgB triple fusions (20 μg) adjuvanted either with MF59 or aluminium hydroxide. Control mice receive MF59 alone or buffer alone. At day 14 mice receive the RrgB triple fusions (20 μg) either unadjuvanted or adjuvanted with MF59 or aluminium hydroxide, and with or without 0.1 μg of influenza vaccine. Control mice receive buffer alone, or influenza vaccine, either unadjuvanted or adjuvanted with MF59 or aluminium hydroxide. At day 28 mice again receive the same composition as at day 14.

For the MN assay, MDCK cells are plated on a 96 well plates at the concentration of 20,000 cells/well. The day after, the mice sera are serially diluted in a 96 well plate and incubated with a fixed amount of influenza virus (300 TCID50/well of each strain) for 1 hour at 37° C. Then the mixture sera/virus is added to plated MDCK cells in presence of trypsin (1:250 final) and incubated at 37° C. After an overnight incubation, infected cells are identified with an ELISA-based assay. MDCK cells are fixed with PFA 2%, permeabilized and labeled with a FITC-conjugated anti-M/NP antibody which is specific for each virus. After 1 hour's incubation cells are stained with a POD-conjugated anti-FITC antibody. After 1 hour the POD substrate is added and the absorbance at 450 nm is evaluated. The absorbance intensity is directly proportional to the number of infected cells. The data obtained for each sample dilutions are interpolated with a 4-parameter fitting curve and the MN titers are expressed as the reciprocal of the serum dilution required to reduce infection by 50%.

For the HI assay sera are analyzed singly and results are represented as the geometric mean titer (GMT). The HI assay is run according to standard procedures using turkey red blood cells. Titers are read as the last serum dilution giving inhibition of hemagglutination. A titer of 10 is assigned to sera that gave a negative result at the first (1:20) dilution tested.

For the OPKA assay data obtained from day 42 sera are tested against serotype 6B pneumococcus. In brief, bacteria opsonized with serial dilutions of heat-inactivated mouse antisera are mixed with baby rabbit complement and phagocytes (differentiated human proleukemia cells) for 1 hour at 37° C., before being plated onto agar. After overnight incubation, surviving colonies are counted and results expressed as percentage of bacteria killed in the OPKA with respect to the control without serum.

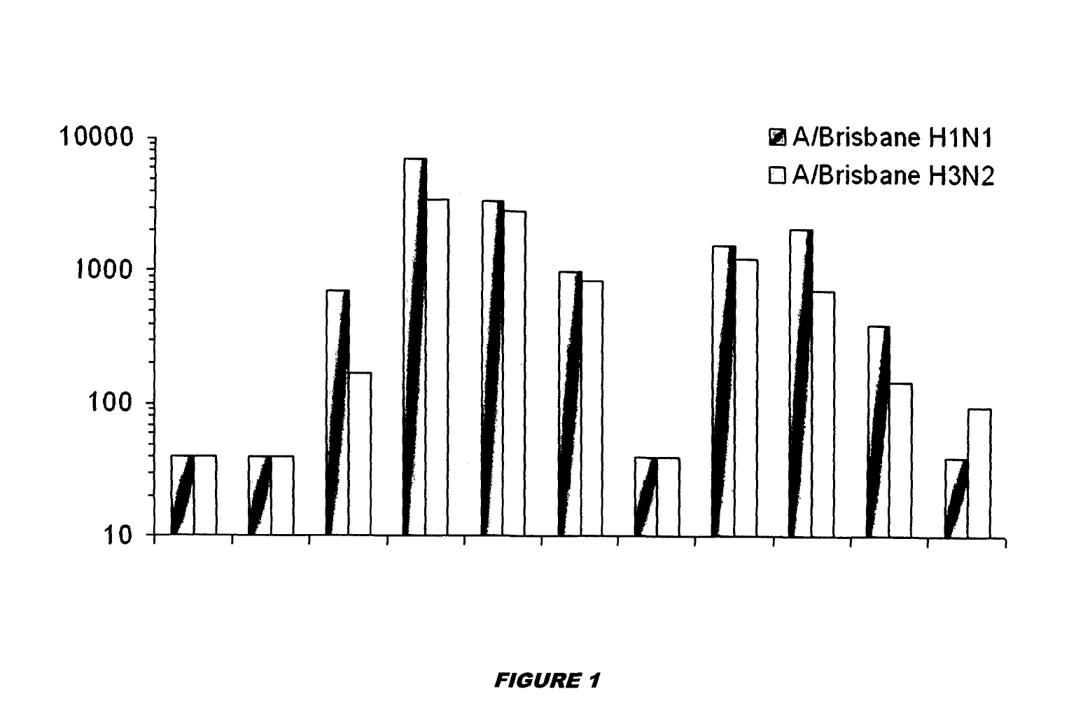

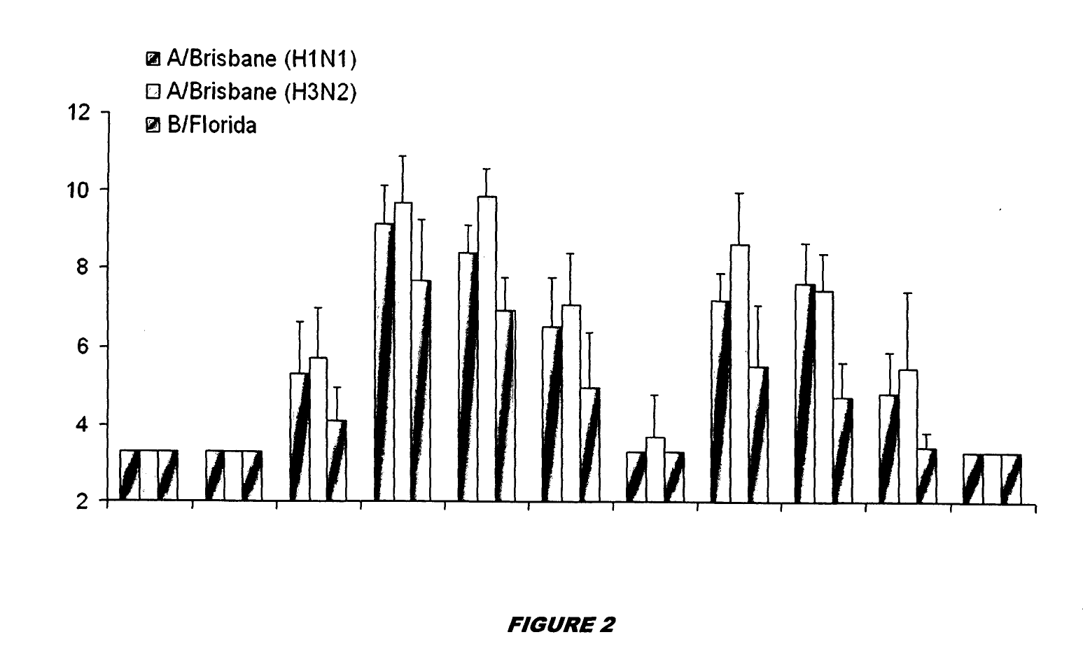

Results with both MN (FIG. 1) and HI (FIG. 2) show overall a better response in the presence of MF59 than with aluminium hydroxide. In general, mice immunized with the combination of influenza vaccine with the ‘321’ fusion show HI and MN titers comparable to the influenza vaccine alone, whereas mice immunized with the combination of influenza vaccine with the ‘213’ fusion show significantly decreased HI and MN titers against the three seasonal strains. Similar results were seen with influenza B virus.

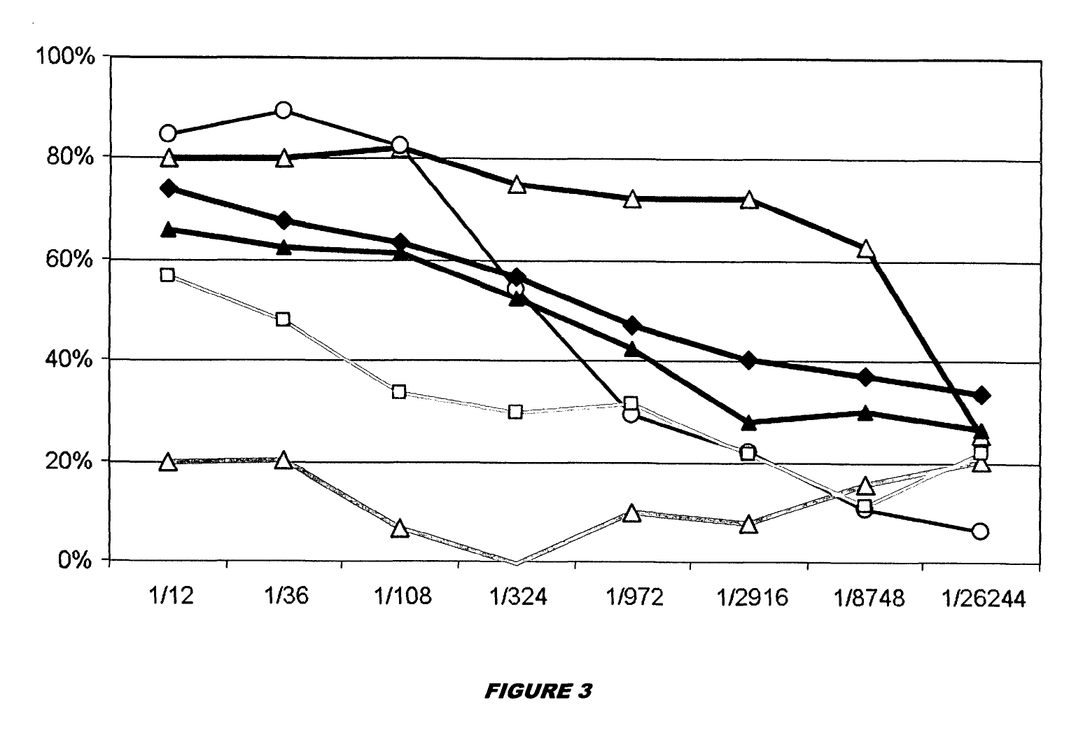

Similarly, the OPA assay (FIG. 3) shows that antibodies raised against with the combination vaccines have similar killing efficacy to antibodies raised against the RrgB fusions alone. In addition, the killing was higher for antisera obtained from combinations including the ‘321’ chimera adjuvanted with MF59.

In conclusion, these preclinical data indicate that combinations of pneumococcal polypeptide antigens and influenza virus antigens can provide an effective immunization strategy to target lower respiratory tract infections. Improved efficacy using an oil-in-water emulsion adjuvant points towards this approach being particularly helpful in infants less than 6 months old [165].

It will be understood that the invention has been described by way of example only and modifications may be made whilst remaining within the scope and spirit of the invention.

### REFERENCES

[1] Hilleman et al. (1981) Rev Infect Dis. 3 Suppl:S31-42.
[2] McCue (1981) J Fam Pract. 13(2):175-7.
[3] Honkanen (1996) Arch Intern Med. 156(2):205-8.
[4] US-2009/0208523.
[5] WO00/62801.
[6] WO00/35481.
[7] Talaat et al. (2001) Vaccine 20:538-44.
[8] WO02/28422.
[9] WO02/067983.
[10] WO02/074336.
[11] WO01/21151.
[12] WO02/097072.
[13] WO2005/113756.
[14] Huckriede et al. (2003) Methods Enzymol 373:74-91.
[15] WO96/37624.
[16] WO98/46262.
[17] WO95/18861.
[18] GenBank sequence GI:325176.
[19] WO01/22992.
[20] Hehme et al. (2004) Virus Res. 103(1-2):163-71.
[21] Treanor et al. (1996) J Infect Dis 173:1467-70.
[22] Keitel et al. (1996) Clin Diagn Lab Immunol 3:507-10.
[23] Gambaryan & Matrosovich (1992) J Virol Methods 39(1-2):111-23.
[24] Mastrosovich et al. (1999) J Virol 73: 1146-55.
[25] Stevens et al. (2006) J Mol Biol 355:1143-55.
[26] Couceiro & Baum (1994) Mem Inst Oswaldo Cruz 89(4):587-91.
[27] Kistner et al. (1998) Vaccine 16:960-8.
[28] Kistner et al. (1999) Dev Biol Stand 98:101-110.
[29] Bruhl et al. (2000) Vaccine 19:1149-58.
[30] Pau et al. (2001) Vaccine 19:2716-21.
[31] http://www.atcc.org/
[32]http://locus.umdnj.edu/
[33]WO03/076601.
[34] WO2005/042728.
[35] WO03/043415.
[36] WO01/85938.
[37] WO2006/108846.
[38] WO97/37000.
[39] Brands et al. (1999) Dev Biol Stand 98:93-100.
[40] Halperin et al. (2002) Vaccine 20:1240-7.
[41] Tree et al. (2001) Vaccine 19:3444-50.
[42] EP-A-1260581 (WO01/64846).
[43] WO2006/071563.
[44] WO2005/113758.
[45] WO97/37001.
[46] WO2006/027698.
[47] EP-B-0870508.
[48] U.S. Pat. No. 5,948,410.
[49] Lundblad (2001) Biotechnology and Applied Biochemistry 34:195-197.
[50] Guidance for Industry: Bioanalytical Method Validation. U.S. Department of Health and Human Services Food and Drug Administration Center for Drug Evaluation and Research (CDER) Center for Veterinary Medicine (CVM). May 2001.
[51] Ji et al. (2002) Biotechniques. 32:1162-7.
[52] Briggs (1991) J Parenter Sci Technol. 45:7-12.
[53] Lahijani et al. (1998) Hum Gene Ther. 9:1173-80.
[54] Lokteff et al. (2001) Biologicals. 29:123-32.
[55] Hoffmann et al. (2002) Vaccine 20:3165-3170.
[56] Subbarao et al. (2003) Virology 305:192-200.
[57] Liu et al. (2003) Virology 314:580-590.
[58] Ozaki et al. (2004) J. Virol. 78:1851-1857.
[59] Webby et al. (2004) Lancet 363:1099-1103.
[60] WO00/60050.
[61] WO01/04333.
[62] U.S. Pat. No. 6,649,372.
[63] Neumann et al. (2005) Proc Natl Acad Sci USA 102:16825-9.
[64] WO01/83794.
[65] Hoffmann et al. (2000) Virology 267(2):310-7.
[66] Kuo et al. (1995) Infect Immun 63:2706-13.
[67] Michon et al. (1998) Vaccine. 16:1732-41.
[68] WO02/091998.
[69] Research Disclosure, 453077 (January 2002).
[70] EP-A-0372501.
[71] EP-A-0378881.
[72] EP-A-0427347.
[73] WO93/17712.
[74] WO94/03208.
[75] WO98/58668.
[76] EP-A-0471177.
[77] WO91/01146.
[78] Falugi et al. (2001) Eur J Immunol 31:3816-3824.
[79] Baraldo et al. (2004) Infect Immun 72(8):4884-7.
[80] EP-A-0594610.
[81] Ruan et al. (1990) J Immunol 145:3379-3384.
[82] WO00/56360.
[83] WO01/72337.
[84] WO00/61761,
[85] WO00/33882
[86] WO2007/071707
[87] WO99/42130.
[88] U.S. Pat. No. 4,761,283.
[89] U.S. Pat. No. 4,356,170.
[90] U.S. Pat. No. 4,882,317.
[91] U.S. Pat. No. 4,695,624.
[92] Mol. Immunol., 1985, 22, 907-919
[93] EP-A-0208375.
[94] Bethell G. S. et al., J. Biol. Chem., 1979, 254:2572-4
[95] Hearn M. T. W., J. Chromatogr., 1981, 218:509-18
[96] WO00/10599.
[97] Geyer et al., Med. Microbiol. Immunol, 165: 171-288 (1979).
[98] U.S. Pat. No. 4,057,685.
[99] U.S. Pat. Nos. 4,673,574; 4,761,283; 4,808,700.
[100] U.S. Pat. No. 4,459,286.
[101] U.S. Pat. No. 4,965,338.
[102] U.S. Pat. No. 4,663,160.
[103] WO2007/000343.
[104] Hoskins et al. (2001) J. Bacteriol. 183:5709-5717.
[105] WO2004/092209.
[106] Kirkham et al. (2006) Infect Immun. 74(1):586-93.
[107] WO2005/108580.
[108] Berry et al. (1999) Infect Immun 67(2):981-5.
[109] U.S. Pat. No. 6,716,432.
[110] WO90/06951.
[111] WO99/03884.
[112] Baba et al. (2002) Infect Immun 70: 107-113.
[113] U.S. Pat. No. 7,217,791
[114] WO2008/061953.
[115] WO01/12219.
[116] Cao et al. (2007) Vaccine 25(27):4996-5005.
[117] WO2005/063283.
[118] WO2003/104272.
[119] WO00/37105.
[120] Adamou et al. (2001) Infect Immun. 69(2):949-58.
[121] Ogunniyi et al. (2007) Infect Immun. 75(1):350-7.
[122] WO98/18930.
[123] WO02/22168.
[124] Wizemann et al. (2001) Infect Immun 69:1593-8.
[125] WO99/53940.
[126] WO02/22167.
[127] WO02/08426.
[128] Briles et al. (2000) J Infect Dis 182:1694-1701.
[129] Talkington et al (1996) Microb Pathog. 21(1):17-22.
[130] WO00/76540.
[131] Bethe et al. (2001) FEMS Microbiol Lett. 205(1):99-104.
[132] WO01/81380.
[133] Brown et al. (2001) Infect Immun 69:6702-6.
[134] Whalan et al. (2005) FEMS Immunol Med Microbiol 43:73-80.
[135] Jomaa et al. (2006) Vaccine. 24(24):5133-9.
[136] WO00/06738.
[137] Barocchi et al. (2006) PNAS USA 103:2857-62.
[138] Bagnoli et al. (2008) J Bacteria 190(15):5480-92.
[139] WO2007/116322.
[140] LeMieux et al. (2006) Infect Immun 74:2453-6.
[141] WO2005/007189.
[142] WO2006/018527.
[143] WO2008/110627.
[144] Langley et al. (2009) Vaccine doi:10.1016/j.vaccine.2009.07.038.
[145] WO2009/055491.
[146] Schickli et al. (2009) Hum Vaccin. 2009 Sep. 28; 5(9).
[147] WO2008/084072.
[148] WO2008/020335.
[149] WO2008/152448.
[150] WO2009/101403.
[151] Edwards (2008) Hum Vaccin 4(6):444-8.
[152] WO2008/061953.
[153] WO90/14837.
[154] Podda & Del Giudice (2003) Expert Rev Vaccines 2:197-203.
[155] Podda (2001) Vaccine 19: 2673-2680.
[156] Vaccine Design: The Subunit and Adjuvant Approach (eds. Powell & Newman) Plenum Press 1995 (ISBN 0-306-44867-X).
[157] Vaccine Adjuvants: Preparation Methods and Research Protocols (Volume 42 of Methods in Molecular Medicine series). ISBN: 1-59259-083-7. Ed. O'Hagan.
[158] Allison & Byars (1992) Res Immunol 143:519-25.
[159] Hariharan et al. (1995) Cancer Res 55:3486-9.
[160] US-2007/014805.
[161] WO95/11700.
[162] U.S. Pat. No. 6,080,725.
[163] WO2006/113373.
[164] WO2005/097181.
[165] Vesikari et al. (2009) Pediatr Infect Dis J 28:563-71.
[166] U.S. Pat. No. 5,057,540.
[167] WO96/33739.
[168] EP-A-0109942.
[169] WO96/11711.
[170] WO00/07621.
[171] Barr et al. (1998) Advanced Drug Delivery Reviews 32:247-271.
[172] Sjolanderet et al. (1998) Advanced Drug Delivery Reviews 32:321-338.
[173] EP-A-0689454.
[174] Johnson et al. (1999) Bioorg Med Chem Lett 9:2273-2278.
[175] Evans et al. (2003) Expert Rev Vaccines 2:219-229.
[176] Meraldi et al. (2003) Vaccine 21:2485-2491.
[177] Pajak et al. (2003) Vaccine 21:836-842.
[178] Kandimalla et al. (2003) Nucleic Acids Research 31:2393-2400.
[179] WO02/26757.
[180] WO99/62923.
[181] Krieg (2003) Nature Medicine 9:831-835.
[182] McCluskie et al. (2002) FEMS Immunology and Medical Microbiology 32:179-185.
[183] WO98/40100.
[184] U.S. Pat. No. 6,207,646.
[185] U.S. Pat. No. 6,239,116.
[186] U.S. Pat. No. 6,429,199.
[187] Kandimalla et al. (2003) Biochemical Society Transactions 31 (part 3):654-658.
[188] Blackwell et al. (2003) J Immunol 170:4061-4068.
[189] Krieg (2002) Trends Immunol 23:64-65.
[190] WO01/95935.
[191] Kandimalla et al. (2003) BBRC 306:948-953.
[192] Bhagat et al. (2003) BBRC 300:853-861.
[193] WO03/035836.
[194] Schellack et al. (2006) Vaccine 24:5461-72.
[195] Lingnau et al. (2007) Expert Rev Vaccines 6:741-6.
[196] WO2004/084938.
[197] WO95/17211.
[198] WO98/42375.
[199] Beignon et al. (2002) Infect Immun 70:3012-3019.
[200] Pizza et al. (2001) Vaccine 19:2534-2541.
[201] Pizza et al. (2000) Int J Med Microbiol 290:455-461.
[202] Scharton-Kersten et al. (2000) Infect Immun 68:5306-5313.
[203] Ryan et al. (1999) Infect Immun 67:6270-6280.
[204] Partidos et al. (1999) Immunol Lett 67:209-216.
[205] Peppoloni et al. (2003) Expert Rev Vaccines 2:285-293.
[206] Pine et al. (2002) J Control Release 85:263-270.
[207] Tebbey et al. (2000) Vaccine 18:2723-34.
[208] Domenighini et al. (1995) Mol Microbiol 15:1165-1167.
[209] WO99/40936.
[210] WO99/44636.
[211] Singh et al] (2001) J Cont Release 70:267-276.
[212] WO99/27960.
[213] U.S. Pat. No. 6,090,406.
[214] U.S. Pat. No. 5,916,588.
[215] EP-A-0626169.
[216] Stanley (2002) Clin Exp Dermatol 27:571-577.
[217] Jones (2003) Curr Opin Investig Drugs 4:214-218.
[218] WO99/11241.
[219] WO94/00153.
[220] WO98/57659.
[221] European patent applications 0835318, 0735898 and 0761231.
[222] Zwijnenburg et al. (2001) J Infect Dis 183:1143-6.
[223] Bodanszky (1993) Principles of Peptide Synthesis (ISBN: 0387564314).
[224] Fields et al. (1997) Meth Enzymol 289: Solid-Phase Peptide Synthesis. ISBN: 0121821900.
[225] Chan & White (2000) Fmoc Solid Phase Peptide Synthesis. ISBN: 0199637245.
[226] Kullmann (1987) Enzymatic Peptide Synthesis. ISBN: 0849368413.
[227] Ibba (1996) Biotechnol Genet Eng Rev 13:197-216.
[228] Gennaro (2000) Remington: The Science and Practice of Pharmacy. 20th edition, ISBN: 0683306472.
[229] Methods In Enzymology (S. Colowick and N. Kaplan, eds., Academic Press, Inc.)
[230] Handbook of Experimental Immunology, Vols. I-IV (D. M. Weir and C. C. Blackwell, eds, 1986, Blackwell Scientific Publications)
[231] Sambrook et al. (2001) Molecular Cloning: A Laboratory Manual, 3rd edition (Cold Spring Harbor Laboratory Press).
[232] Handbook of Surface and Colloidal Chemistry (Birdi, K. S. ed., CRC Press, 1997)
[233] Ausubel et al. (eds) (2002) Short protocols in molecular biology, 5th edition (Current Protocols).
[234] Molecular Biology Techniques: An Intensive Laboratory Course, (Ream et al., eds., 1998, Academic Press)
[235] PCR (Introduction to Biotechniques Series), 2nd ed. (Newton & Graham eds., 1997, Springer Verlag)
[236] Geysen et al. (1984) PNAS USA 81:3998-4002.
[237] Carter (1994) Methods Mol Biol 36:207-23.
[238] Jameson, B A et al. 1988, CABIOS 4(1):181-186.
[239] Raddrizzani & Hammer (2000) Brief Bioinform 1(2):179-89.
[240] Bublil et al. (2007) Proteins 68(1):294-304.
[241] De Lalla et al. (1999) J Immunol. 163:1725-29.
[242] Kwok et al. (2001) Trends Immunol 22:583-88.
[243] Brusic et al. (1998) Bioinformatics 14(2):121-30
[244] Meister et al. (1995) Vaccine 13(6):581-91.
[245] Roberts et al. (1996) AIDS Res Hum Retroviruses 12(7):593-610.
[246] Maksyutov & Zagrebelnaya (1993) Comput Appl Biosci 9(3):291-7.
[247] Feller & de la Cruz (1991) Nature 349(6311):720-1.
[248] Hopp (1993) Peptide Research 6:183-190.
[249] Welling et al. (1985) FEBS Lett. 188:215-218.
[250] Davenport et al. (1995) Immunogenetics 42:392-297.
[251] Tsurui & Takahashi (2007) J Pharmacol Sci. 105(4):299-316.
[252] Tong et al. (2007) Brief Bioinform. 8(2):96-108.
[253] Schirle et al. (2001) J Immunol Methods. 257(1-2):1-16.
[254] Chen et al. (2007) Amino Acids 33(3):423-8.
[255] Current Protocols in Molecular Biology (F. M. Ausubel et al., eds., 1987) Supplement 30
[256] Smith & Waterman (1981) Adv. Appl. Math. 2: 482-489.

1. An immunogenic composition comprising (a) an influenza virus immunogen and a pneumococcal immunogen, (b) a RSV immunogen and a pneumococcal immunogen, or (c) an influenza virus immunogen, a RSV immunogen and a pneumococcal immunogen, wherein the pneumococcal immunogen comprises at least one pneumococcal polypeptide.

2. The composition of claim 1, including an adjuvant.

3. The composition of claim 2, wherein the adjuvant comprises an oil-in-water emulsion.

4. The composition of claim 1, including a group B streptococcus immunogen.

5. The composition of claim 1, wherein the composition has a unit dose volume of 0.5 ml.

6. The composition of claim 1, wherein the influenza virus immunogen comprises hemagglutinins from a H1N1 influenza A virus, a H3N2 influenza A virus, a B/Victoria/2/87-like influenza B virus and a B/Yamagata/16/88-like influenza B virus.

7. A process for preparing the immunogenic composition of claim 1, comprising a step of admixing two or more of an influenza virus immunogen, a pneumococcal immunogen, and/or a RSV immunogen, wherein the pneumococcal immunogen comprises a pneumococcal polypeptide.

8. The process of claim 7, including a step of admixing a GBS immunogen.

9. The process of claim 7, wherein the process gives a composition with a unit dose volume of 0.5 ml.

10. A kit comprising (i) a first kit component comprising an influenza virus immunogen and (ii) a second kit component comprising a pneumococcal immunogen, wherein the pneumococcal immunogen comprises at least one pneumococcal polypeptide.

11. The kit of claim 10, wherein the second kit component is in dried form.

12. The kit of claim 10, wherein the first kit component includes an adjuvant.

13. The composition claim 1, wherein the influenza immunogen is a split virus vaccine or purified influenza virus surface antigen vaccine including a hemagglutinin from two influenza A strains (H1N1 and H3N2) and one influenza B strain.

14. The composition claim 1, wherein the pneumococcal immunogen comprises (a) a first amino acid sequence comprising an amino acid sequence (i) having at least 75% sequence identity to SEQ ID NO: 1 and/or (ii) consisting of a fragment of at least 7 contiguous amino acids from SEQ ID NO: 1; (b) a second amino acid sequence comprising an amino acid sequence (i) having at least 75% sequence identity to SEQ ID NO: 2 and/or (ii) consisting of a fragment of at least 7 contiguous amino acids from SEQ ID NO: 2; and (c) a third amino acid sequence, comprising an amino acid sequence (i) having at least 75% sequence identity to SEQ ID NO: 3 and/or (ii) consisting of a fragment of at least 7 contiguous amino acids from SEQ ID NO: 3.

15. A method for raising an immune response in a mammal comprising the step of administering to the mammal an effective amount of the immunogenic composition of claim 1.


SEQUENCE LISTING (72 sequences)

SEQ ID NO: 1 - protein, 665 aa, Streptococcus pneumoniae
MKSINKFLTMLAALLLTASSLFSAATVFAAGTTTTSVTVHKLLATDGDMDKIANELETGNYAGNKVGVLPANAKEIAGVMFVWTNTNNEIIDENGQTLGVNIDPQTFKLSGAMPATAMKKLTEAEGAKFNTANLPAAKYKIYEIHSLSTYVGEDGATLTGSKAVPIEIELPLNDVVDAHVYPKNTEAKPKIDKDFKGKANPDTPRVDKDTPVNHQVGDVVEYEIVTKIPALANYATANWSDRMTEGLAFNKGTVKVTVDDVALEAGDYALTEVATGFDLKLTDAGLAKVNDQNAEKTVKITYSATLNDKAIVEVPESNDVTFNYGNNPDHGNTPKPNKPNENGDLTLTKTWVDATGAPIPAGAEATFDLVNAQTGKVVQTVTLTTDKNTVTVNGLDKNTEYKFVERSIKGYSADYQEITTAGEIAVKNWKDENPKPLDPTEPKVVTYGKKFVKVNDKDNRLAGAEFVIANADNAGQYLARKADKVSQEEKQLVVTTKDALDRAVAAYNALTAQQQTQQEKEKVDKAQAAYNAAVIAANNAFEWVADKDNENVVKLVSDAQGRFEITGLLAGTYYLEETKQPAGYALLTSRQKFEVTATSYSATGQGIEYTAGSGKDDATKVVNKKITIPQTGGIGTIIFAVAGAAIMGIAVYAYVKNNKDEDQLA

SEQ ID NO: 2 - protein, 644 aa, Streptococcus pneumoniae
MKSINKFLTMLAALLLTASSLFSAATVFAADNVSTAPDAVTKTLTIHKLLLSEDDLKTWDTNGPKGYDGTQSSLKDLTGVVAEEIPNVYFELQKYNLTDGKEKENLKDDSKWTTVHGGLTTKDGLKIETSTLKGVYRIREDRTKTTYVGPNGQVLTGSKAVPALVTLPLVNNNGTVIDAHVFPKNSYNKPVVDKRIADTLNYNDQNGLSIGTKIPYVVNTTIPSNATFATSFWSDEMTEGLTYNEDVTITLNNVAMDQADYEVTKGNNGFNLKLTEAGLAKINGKDADQKIQITYSATLNSLAVADIPESNDITYHYGNHQDHGNTPKPTKPNNGQITVTKTWDSQPAPEGVKATVQLVNAKTGEKVGAPVELSENNWTYTWSGLDNSIEYKVEEEYNGYSAEYTVESKGKLGVKNWKDNNPAPINPEEPRVKTYGKKFVKVDQKDTRLENAQFVVKKADSNKYIAFKSTAQQAADEKAAATAKQKLDAAVAAYTNAADKQAAQALVDQAQQEYNVAYKEAKFGYVEVAGKDEAMVLTSNTDGQFQISGLAAGTYKLEEIKAPEGFAKIDDVEFVVGAGSWNQGEFNYLKDVQKNDATKVVNKKITIPQTGGIGTIIFAVAGAAIMGIAVYAYVKNNKDEDQLA

SEQ ID NO: 3 - protein, 654 aa, Streptococcus pneumoniae
MKSINKFLTILAALLLTVSSLFSAATVFAAEQKTKTLTVHKLLMTDQELDAWNSDAITTAGYDGSQNFEQFKQLQGVPQGVTEISGVAFELQSYTGPQGKEQENLTNDAVWTAVNKGVTTETGVKFDTEVLQGTYRLVEVRKESTYVGPNGKVLTGMKAVPALITLPLVNQNGVVENAHVYPKNSEDKPTATKTFDTAAGFVDPGEKGLAIGTKVPYIVTTTIPKNSTLATAFWSDEMTEGLDYNGDVVVNYNGQPLDNSHYTLEAGHNGFILKLNEKGLEAINGKDAEATITLKYTATLNALAVADVPEANDVTFHYGNNPGHGNTPKPNKPKNGELTITKTWADAKDAPIAGVEVTFDLVNAQTGEVVKVPGHETGIVLNQTNNWTFTATGLDNNTEYKFVERTIKGYSADYQTITETGKIAVKNWKDENPEPINPEEPRVKTYGKKFVKVDQKDERLKEAQFVVKNEQGKYLALKSAAQQAVNEKAAAEAKQALDAAIAAYTNAADKNAAQAVVDAAQKTYNDNYRAARFGYVEVERKEDALVLTSNTDGQFQISGLAAGSYTLEETKAPEGFAKLGDVKFEVGAGSWNQGDFNYLKDVQKNDATKVVNKKITIPQTGGIGTIIFAVAGAVIMGIAVYAYVKNNKDEDQLA

SEQ ID NO: 4 - protein, 604 aa, Streptococcus pneumoniae
AATVFAAGTTTTSVTVHKLLATDGDMDKIANELETGNYAGNKVGVLPANAKEIAGVMFVWTNTNNEIIDENGQTLGVNIDPQTFKLSGAMPATAMKKLTEAEGAKFNTANLPAAKYKIYEIHSLSTYVGEDGATLTGSKAVPIEIELPLNDVVDAHVYPKNTEAKPKIDKDFKGKANPDTPRVDKDTPVNHQVGDVVEYEIVTKIPALANYATANWSDRMTEGLAFNKGTVKVTVDDVALEAGDYALTEVATGFDLKLTDAGLAKVNDQNAEKTVKITYSATLNDKAIVEVPESNDVTFNYGNNPDHGNTPKPNKPNENGDLTLTKTWVDATGAPIPAGAEATFDLVNAQTGKVVQTVTLTTDKNTVTVNGLDKNTEYKFVERSIKGYSADYQEITTAGEIAVKNWKDENPKPLDPTEPKVVTYGKKFVKVNDKDNRLAGAEFVIANADNAGQYLARKADKVSQEEKQLVVTTKDALDRAVAAYNALTAQQQTQQEKEKVDKAQAAYNAAVIAANNAFEWVADKDNENVVKLVSDAQGRFEITGLLAGTYYLEETKQPAGYALLTSRQKFEVTATSYSATGQGIEYTAGSGKDDATKVVNKKIT

SEQ ID NO: 5 - protein, 583 aa, Streptococcus pneumoniae
AATVFAADNVSTAPDAVTKTLTIHKLLLSEDDLKTWDTNGPKGYDGTQSSLKDLTGVVAEEIPNVYFELQKYNLTDGKEKENLKDDSKWTTVHGGLTTKDGLKIETSTLKGVYRIREDRTKTTYVGPNGQVLTGSKAVPALVTLPLVNNNGTVIDAHVFPKNSYNKPVVDKRIADTLNYNDQNGLSIGTKIPYVVNTTIPSNATFATSFWSDEMTEGLTYNEDVTITLNNVAMDQADYEVTKGNNGFNLKLTEAGLAKINGKDADQKIQITYSATLNSLAVADIPESNDITYHYGNHQDHGNTPKPTKPNNGQITVTKTWDSQPAPEGVKATVQLVNAKTGEKVGAPVELSENNWTYTWSGLDNSIEYKVEEEYNGYSAEYTVESKGKLGVKNWKDNNPAPINPEEPRVKTYGKKFVKVDQKDTRLENAQFVVKKADSNKYIAFKSTAQQAADEKAAATAKQKLDAAVAAYTNAADKQAAQALVDQAQQEYNVAYKEAKFGYVEVAGKDEAMVLTSNTDGQFQISGLAAGTYKLEEIKAPEGFAKIDDVEFVVGAGSWNQGEFNYLKDVQKNDATKVVNKKIT

SEQ ID NO: 6 - protein, 587 aa, Streptococcus pneumoniae
AEQKTKTLTVHKLLMTDQELDAWNSDAITTAGYDGSQNFEQFKQLQGVPQGVTEISGVAFELQSYTGPQGKEQENLTNDAVWTAVNKGVTTETGVKFDTEVLQGTYRLVEVRKESTYVGPNGKVLTGMKAVPALITLPLVNQNGVVENAHVYPKNSEDKPTATKTFDTAAGFVDPGEKGLAIGTKVPYIVTTTIPKNSTLATAFWSDEMTEGLDYNGDVVVNYNGQPLDNSHYTLEAGHNGFILKLNEKGLEAINGKDAEATITLKYTATLNALAVADVPEANDVTFHYGNNPGHGNTPKPNKPKNGELTITKTWADAKDAPIAGVEVTFDLVNAQTGEVVKVPGHETGIVLNQTNNWTFTATGLDNNTEYKFVERTIKGYSADYQTITETGKIAVKNWKDENPEPINPEEPRVKTYGKKFVKVDQKDERLKEAQFVVKNEQGKYLALKSAAQQAVNEKAAAEAKQALDAAIAAYTNAADKNAAQAVVDAAQKTYNDNYRAARFGYVEVERKEDALVLTSNTDGQFQISGLAAGSYTLEETKAPEGFAKLGDVKFEVGAGSWNQGDFNYLKDVQKNDATKVVNKKIT

SEQ ID NO: 7 - protein, 132 aa, Streptococcus pneumoniae
MVMTDPIADFLTRIRNANQAKHEVLEVPASNIKKGIAEILKREGFVKNVEIIEDDKQGVIRVFLKYGPNGEKVITNLKRVSKPGLRVYKKREDLPKVLNGLGIAILSTSEGLLTDKEARQKNVGGEVIAYVW

SEQ ID NO: 8 - protein, 279 aa, Streptococcus pneumoniae
MGIALENVNFIYQEGTPLASAALSDVSLTIEDGSYTALIGHTGSGKSTILQLLNGLLVPSQGSVRVFDTLITSTSKNKDIRQIRKQVGLVFQFAENQIFEETVLKDVAFGPQNFGVSEEDAVKTAREKLALVGIDESLFDRSPFELSGGQMRRVAIAGILAMEPSILVLDEPTAGLDPLGRKELMTLFKKLHQSGMTIVLVTHLMDDVAEYANQVYVMEKGRLVKGGKPSDVFQDVVFMEEVQLGVPKITAFCKRLADRGVSFKRLPIKIEEFKESLNG

SEQ ID NO: 9 - protein, 321 aa, Streptococcus pneumoniae
MKTSLKLYFTALVASFLLLLGACSTNSSTSQTETSSSAPTEITIKSSLDEVKLSKVPEKIVTFDLGAADTIRALGFEKNIVGMPTKTVPTYLKDLVGTVKNVGSMKEPDLEAIAALEPDLIIASPRTQKFVDKFKEIAPTVLFQASKDDYWTSTKANIESLASAFGETSTQKAKEELAKLDKSIQEVATKNESSDKKALAILLNEGKMAAFGAKSRFSFLYQTLKFKPTDTKFEDSRHGQEVSFESVKEINPDILFVINRTLAIGGDNSSNDGVLENALIAETPAAKNGKIIQLTPDLWYLSGGGLESTKLMIEDIQKALK

SEQ ID NO: 10 - protein, 339 aa, Streptococcus pneumoniae
MHAKMRNKKQINLGIIFVICLGLLITIFLSLKLGTKEINIRDFLAAFGMGNTNDDFIKSIIYKRIPRTIFAILAGSSLAISGVLMQSVTRNPIADPGILGINTGASLSVVIGLSFLGISSSISHISFAIIGGLVSAIFVYAIAVSGKAGLTPIKLALSGTCVSMALSSFVSFLILPNNNVLDKFRFWQIGSLGAATLSSISTLLPFIILGHLIAIFISSDLNALAMGDEMAVGLGVNVNRIRSLAIIASVLLCSSITAIGGPIGFVGLIVPHFCGLFISKDIRTMTISSAFIGAELLLICDIIGRMLGKPGEIEVGIITAIIGGPVLIYVTMKNRGVNT

SEQ ID NO: 11 - protein, 1801 aa, Streptococcus pneumoniae
MASAATVFAAGTTTTSVTVHKLLATDGDMDKIANELETGNYAGNKVGVLPANAKEIAGVMFVWTNTNNEIIDENGQTLGVNIDPQTFKLSGAMPATAMKKLTEAEGAKFNTANLPAAKYKIYEIHSLSTYVGEDGATLTGSKAVPIEIELPLNDVVDAHVYPKNTEAKPKIDKDFKGKANPDTPRVDKDTPVNHQVGDVVEYEIVTKIPALANYATANWSDRMTEGLAFNKGTVKVTVDDVALEAGDYALTEVATGFDLKLTDAGLAKVNDQNAEKTVKITYSATLNDKAIVEVPESNDVTFNYGNNPDHGNTPKPNKPNENGDLTLTKTWVDATGAPIPAGAEATFDLVNAQTGKVVQTVTLTTDKNTVTVNGLDKNTEYKFVERSIKGYSADYQEITTAGEIAVKNWKDENPKPLDPTEPKVVTYGKKFVKVNDKDNRLAGAEFVIANADNAGQYLARKADKVSQEEKQLVVTTKDALDRAVAAYNALTAQQQTQQEKEKVDKAQAAYNAAVIAANNAFEWVADKDNENVVKLVSDAQGRFEITGLLAGTYYLEETKQPAGYALLTSRQKFEVTATSYSATGQGIEYTAGSGKDDATKVVNKKITGSGSGGGGAATVFAADNVSTAPDAVTKTLTIHKLLLSEDDLKTWDTNGPKGYDGTQSSLKDLTGVVAEEIPNVYFELQKYNLTDGKEKENLKDDSKWTTVHGGLTTKDGLKIETSTLKGVYRIREDRTKTTYVGPNGQVLTGSKAVPALVTLPLVNNNGTVIDAHVFPKNSYNKPVVDKRIADTLNYNDQNGLSIGTKIPYVVNTTIPSNATFATSFWSDEMTEGLTYNEDVTITLNNVAMDQADYEVTKGNNGFNLKLTEAGLAKINGKDADQKIQITYSATLNSLAVADIPESNDITYHYGNHQDHGNTPKPTKPNNGQITVTKTWDSQPAPEGVKATVQLVNAKTGEKVGAPVELSENNWTYTWSGLDNSIEYKVEEEYNGYSAEYTVESKGKLGVKNWKDNNPAPINPEEPRVKTYGKKFVKVDQKDTRLENAQFVVKKADSNKYIAFKSTAQQAADEKAAATAKQKLDAAVAAYTNAADKQAAQALVDQAQQEYNVAYKEAKFGYVEVAGKDEAMVLTSNTDGQFQISGLAAGTYKLEEIKAPEGFAKIDDVEFVVGAGSWNQGEFNYLKDVQKNDATKVVNKKITLGGSGGGGAEQKTKTLTVHKLLMTDQELDAWNSDAITTAGYDGSQNFEQFKQLQGVPQGVTEISGVAFELQSYTGPQGKEQENLTNDAVWTAVNKGVTTETGVKFDTEVLQGTYRLVEVRKESTYVGPNGKVLTGMKAVPALIILPLVNQNGVVENAHVYPKNSEDKPTATKTFDTAAGFVDPGEKGLAIGTKVPYIVTTTIPKNSTLATAFWSDEMTEGLDYNGDVVVNYNGQPLDNSHYTLEAGHNGFILKLNEKGLEAINGKDAEATITLKYTATLNALAVADVPEANDVTFHYGNNPGHGNTPKPNKPKNGELTITKTWADAKDAPIAGVEVTFDLVNAQTGEVVKVPGHETGIVLNQTNNWTFTATGLDNNTEYKFVERTIKGYSADYQTITETGKIAVKNWKDENPEPINPEEPRVKTYGKKFVKVDQKDERLKEAQFVVKNEQGKYLALKSAAQQAVNEKAAAEAKQALDAAIAAYTNAADKNAAQAVVDAAQKTYNDNYRAARFGYVEVERKEDALVLTSNTDGQFQISGLAAGSYTLEETKAPEGFAKLGDVKFEVGAGSWNQGDFNYLKDVQKNDATKVVNKKITLGHHHHHH

SEQ ID NO: 12 - protein, 324 aa, Streptococcus pneumoniae
MRKKLFLTSAAILWAVTAMNSVHAATDVQKVIDETYVQPEYVLGSSLSEDQKNQTLKKLGYNASTDTKELKTMTPDVYSKIMNVANDSSLQLYSSAKIQKLGDKSPLEVKIETPENITKVTQDMYRNAAVTLGVEHAKITVAAPIPVTGESALAGIYYSLEANGAKVPQANKDLAQEELKALSDINAENKDKSGYDANKLNVALADIKSGLAKAKESKGNLTEEDIRKIVEDTLKNYKLDQVITGNQINIIINFALNLSKSDILSNADFTKTLNDLKQSIVSQAGDSFKNINLNFDSDKALEDGGNFLSSLWQALVNFFKSFGS

SEQ ID NO: 13 - protein, 1801 aa, Streptococcus pneumoniae
MASAATVFAAGTTTTSVTVHKLLATDGDMDKIANELETGNYAGNKVGVLPANAKEIAGVMFVWTNTNNEIIDENGQTLGVNIDPQTFKLSGAMPATAMKKLTEAEGAKFNTANLPAAKYKIYEIHSLSTYVGEDGATLTGSKAVPIEIELPLNDVVDAHVYPKNTEAKPKIDKDFKGKANPDTPRVDKDTPVNHQVGDVVEYEIVTKIPALANYATANWSDRMTEGLAFNKGTVKVTVDDVALEAGDYALTEVATGFDLKLTDAGLAKVNDQNAEKTVKITYSATLNDKAIVEVPESNDVTFNYGNNPDHGNTPKPNKPNENGDLTLTKTWVDATGAPIPAGAEATFDLVNAQTGKVVQTVTLTTDKNTVTVNGLDKNTEYKFVERSIKGYSADYQEITTAGEIAVKNWKDENPKPLDPTEPKVVTYGKKFVKVNDKDNRLAGAEFVIANADNAGQYLARKADKVSQEEKQLVVTTKDALDRAVAAYNALTAQQQTQQEKEKVDKAQAAYNAAVIAANNAFEWVADKDNENVVKLVSDAQGRFEITGLLAGTYYLEETKQPAGYALLTSRQKFEVTATSYSATGQGIEYTAGSGKDDATKVVNKKITGSGSGGGGAEQKTKTLTVHKLLMTDQELDAWNSDAITTAGYDGSQNFEQFKQLQGVPQGVTEISGVAFELQSYTGPQGKEQENLTNDAVWTAVNKGVTTETGVKFDTEVLQGTYRLVEVRKESTYVGPNGKVLTGMKAVPALIILPLVNQNGVVENAHVYPKNSEDKPTATKTFDTAAGFVDPGEKGLAIGTKVPYIVTTTIPKNSTLATAFWSDEMTEGLDYNGDVVVNYNGQPLDNSHYTLEAGHNGFILKLNEKGLEAINGKDAEATITLKYTATLNALAVADVPEANDVTFHYGNNPGHGNTPKPNKPKNGELTITKTWADAKDAPIAGVEVTFDLVNAQTGEVVKVPGHETGIVLNQTNNWTFTATGLDNNTEYKFVERTIKGYSADYQTITETGKIAVKNWKDENPEPINPEEPRVKTYGKKFVKVDQKDERLKEAQFVVKNEQGKYLALKSAAQQAVNEKAAAEAKQALDAAIAAYTNAADKNAAQAVVDAAQKTYNDNYRAARFGYVEVERKEDALVLTSNTDGQFQISGLAAGSYTLEETKAPEGFAKLGDVKFEVGAGSWNQGDFNYLKDVQKNDATKVVNKKITLGGSGGGGAATVFAADNVSTAPDAVTKTLTIHKLLLSEDDLKTWDTNGPKGYDGTQSSLKDLTGVVAEEIPNVYFELQKYNLTDGKEKENLKDDSKWTTVHGGLTTKDGLKIETSTLKGVYRIREDRTKTTYVGPNGQVLTGSKAVPALVTLPLVNNNGTVIDAHVFPKNSYNKPVVDKRIADTLNYNDQNGLSIGTKIPYVVNTTIPSNATFATSFWSDEMTEGLTYNEDVTITLNNVAMDQADYEVTKGNNGFNLKLTEAGLAKINGKDADQKIQITYSATLNSLAVADIPESNDITYHYGNHQDHGNTPKPTKPNNGQITVTKTWDSQPAPEGVKATVQLVNAKTGEKVGAPVELSENNWTYTWSGLDNSIEYKVEEEYNGYSAEYTVESKGKLGVKNWKDNNPAPINPEEPRVKTYGKKFVKVDQKDTRLENAQFVVKKADSNKYIAFKSTAQQAADEKAAATAKQKLDAAVAAYTNAADKQAAQALVDQAQQEYNVAYKEAKFGYVEVAGKDEAMVLTSNTDGQFQISGLAAGTYKLEEIKAPEGFAKIDDVEFVVGAGSWNQGEFNYLKDVQKNDATKVVNKKITLGHHHHHH

SEQ ID NO: 14 - protein, 2144 aa, Streptococcus pneumoniae
MKKSTVLSLTTAAVILAAYAPNEVVLADTSSSEDALSISDKEKVVVDKETENKEKHKDIHNAIETSKDTEEKKTTIIEEKEVVSKNPVIDTKTSNEEAKIKEENSNQSQGDHTDSFVNKNTENPKKEDKVVYIAEFKDKESGEKAIKGLSNLKNTKVLYTYDRIFNGSAIETTPDNLDKIKQIEGISSIERAQKVQPMMNHARKEIGVEEAIDYLKSINAPFGKNFDGRGMVISNIDTGTDYRHKAMRIDDDAKASMRFKKEDLKGTDKNYWLSDKIPHAFNYYNGGKITVEKYDDGRDYFDPHGMHIAGILAGNDTEQDIKNFNGIDGIAPNAQIFSYKMYSDAGSGFAGDETMFHAIEDSIKHNVDVVSVSSGFTGTGLVGEKYWQAIRALRKAGIPMVVATGNYATSASSSSWDLVANNHLKMTDTGNVTRTAAHEDAIAVASAKNQTVEFDKVNIGGESFKYRNIGAFFDKNKITTNEDGTKAPSKLKFVYIGKGQDQDLIGLDLRGKIAVMDRIYTKDLKNAFKKAMDKGARAIMVVNTVNYYNRDNWTELPAMGYEADEGTKSQVFSISGDDGVKLWNMINPDKKTEVKRNNKEDFKDKLEQYYPIDMESFNSNKPNVGDEKEIDFKFAPDTDKELYKEDIIVPAGSTSWGPRIDLLLKPDVSAPGKNIKSTLNVINGKSTYGYMSGTSMATPIVAASTVLIRPKLKEMLERPVLKNLKGDDKIDLTSLTKIALQNTARPMMDATSWKEKSQYFASPRQQGAGLINVANALRNEVVATFKNTDSKGLVNSYGSISLKEIKGDKKYFTIKLHNTSNRPLTFKVSASAITTDSLTDRLKLDETYKDEKSPDGKQIVPEIHPEKVKGANITFEHGTFTIGANSSFDLNAVINVGEAKNKNKFVESFIHFESVEEMEALNSNGKKINFQPSLSMPLMGFAGNWNHEPILDKWAWEEGSRSKTLGGYDDDGKPKIPGTLNKGIGGEHGIDKFNPAGVIQNRKDKNTTSLDQNPELFAFNNQGINAPSSSGSKIANIYPLDSNGNPQDAQLERGLTPSPLVLRSAEEGLISIVNTNKEGENQRDLKVISREHFIRGILNSKSNDAKGIKSSKLKVWGDLKWDGLIYNPRGREENAPESKDNQDPATKIRGQFEPIAEGQYFYKFKYRLTKDYPWQVSYIPVKIDNTAPKIVSVDFSNPEKIKLITKDTYHKVKDQYKNETLFARDQKEHPEKFDEIANEVWYAGAALVNEDGEVEKNLEVTYAGEGQGRNRKLDKDGNTIYEIKGAGDLRGKIIEVIALDGSSNFTKIHRIKFADQADEKGMISYYLVDPDKDASKYEKLGEISEDKLKNAKSPEENTNNNQAKDEDSKPDEKSSVEGEASLEINKTISTIREFENKDLKKLIKKKFREVNDFTSETGKRIEEYDYKYDDKGNIIAYDDGSALQYETEKFDEIKSKIYGVLSPSKDGHFEILGKISNVSKNAKVYYGNSYKSIEIKATKYDSHSKTMIFDLYANINDIVDGLAFAGDMRLFVKDDNQIKAETKIRMPEKNKETKAEYPYVSSYGNVIELGEGDLSKNKPDNLTKMESGKIYSDSEKQQYLLKDNIILRKGYALKVTTYNPGKTDMLEGNGVYSKEDIAKIQKANPNLRVLSETTIYADSRNVEDGRSTQAVLMSALDGFNIIRYQVFTFKMNDKGEAIDKDGNLVTDSSKLVLFGKDDKEYTGEDKSNVEAIKEDGSMLFIDTKPVNLSMDKNYFNPSKSNKIYVRNPEFYLRGKISDKGGFNWELRVNESVVDNYLIYGDLHIDNTRDFNIKLNVKDGDIMDWGMKDYKANGFPDKVTDMDGNVYLQTGYSDLNAKAVGVHYQFLYDNVKPEVNIDPKGNTSIEYADGKSVVFNINDKRNNGFDGEIQEQHIYVNGKEYTSFDDIKQITDKTLNIKIVVKDFARNTTVKEFILNKDTGEVSELKPHRVTVTIQNGKEMSSTIVSEEDFILPVYKGELEKGYQFDGWEISGFEGKKDAGYVINLSKDTFIKPVFKKIEEKKEEENKPTFDVSKKKDNPQVNHSQLNESHRKEDLQREDHSQKSDSTKDVTATVLDKNNISSKSTTNNPNKLPKTGTASGAQTLLAAGIMFIVGIFLGLKKKNQD

SEQ ID NO: 15 - protein, 1801 aa, Streptococcus pneumoniae
MASAEQKTKTLTVHKLLMTDQELDAWNSDAITTAGYDGSQNFEQFKQLQGVPQGVTEISGVAFELQSYTGPQGKEQENLTNDAVWTAVNKGVTTETGVKFDTEVLQGTYRLVEVRKESTYVGPNGKVLTGMKAVPALIILPLVNQNGVVENAHVYPKNSEDKPTATKTFDTAAGFVDPGEKGLAIGTKVPYIVTTTIPKNSTLATAFWSDEMTEGLDYNGDVVVNYNGQPLDNSHYTLEAGHNGFILKLNEKGLEAINGKDAEATITLKYTATLNALAVADVPEANDVTFHYGNNPGHGNTPKPNKPKNGELTITKTWADAKDAPIAGVEVTFDLVNAQTGEVVKVPGHETGIVLNQTNNWTFTATGLDNNTEYKFVERTIKGYSADYQTITETGKIAVKNWKDENPEPINPEEPRVKTYGKKFVKVDQKDERLKEAQFVVKNEQGKYLALKSAAQQAVNEKAAAEAKQALDAAIAAYTNAADKNAAQAVVDAAQKTYNDNYRAARFGYVEVERKEDALVLTSNTDGQFQISGLAAGSYTLEETKAPEGFAKLGDVKFEVGAGSWNQGDFNYLKDVQKNDATKVVNKKITGSGSGGGGAATVFAADNVSTAPDAVTKTLTIHKLLLSEDDLKTWDTNGPKGYDGTQSSLKDLTGVVAEEIPNVYFELQKYNLTDGKEKENLKDDSKWTTVHGGLTTKDGLKIETSTLKGVYRIREDRTKTTYVGPNGQVLTGSKAVPALVTLPLVNNNGTVIDAHVFPKNSYNKPVVDKRIADTLNYNDQNGLSIGTKIPYVVNTTIPSNATFATSFWSDEMTEGLTYNEDVTITLNNVAMDQADYEVTKGNNGFNLKLTEAGLAKINGKDADQKIQITYSATLNSLAVADIPESNDITYHYGNHQDHGNTPKPTKPNNGQITVTKTWDSQPAPEGVKATVQLVNAKTGEKVGAPVELSENNWTYTWSGLDNSIEYKVEEEYNGYSAEYTVESKGKLGVKNWKDNNPAPINPEEPRVKTYGKKFVKVDQKDTRLENAQFVVKKADSNKYIAFKSTAQQAADEKAAATAKQKLDAAVAAYTNAADKQAAQALVDQAQQEYNVAYKEAKFGYVEVAGKDEAMVLTSNTDGQFQISGLAAGTYKLEEIKAPEGFAKIDDVEFVVGAGSWNQGEFNYLKDVQKNDATKVVNKKITLGGSGGGGAATVFAAGTTTTSVTVHKLLATDGDMDKIANELETGNYAGNKVGVLPANAKEIAGVMFVWTNTNNEIIDENGQTLGVNIDPQTFKLSGAMPATAMKKLTEAEGAKFNTANLPAAKYKIYEIHSLSTYVGEDGATLTGSKAVPIEIELPLNDVVDAHVYPKNTEAKPKIDKDFKGKANPDTPRVDKDTPVNHQVGDVVEYEIVTKIPALANYATANWSDRMTEGLAFNKGTVKVTVDDVALEAGDYALTEVATGFDLKLTDAGLAKVNDQNAEKTVKITYSATLNDKAIVEVPESNDVTFNYGNNPDHGNTPKPNKPNENGDLTLTKTWVDATGAPIPAGAEATFDLVNAQTGKVVQTVTLTTDKNTVTVNGLDKNTEYKFVERSIKGYSADYQEITTAGEIAVKNWKDENPKPLDPTEPKVVTYGKKFVKVNDKDNRLAGAEFVIANADNAGQYLARKADKVSQEEKQLVVTTKDALDRAVAAYNALTAQQQTQQEKEKVDKAQAAYNAAVIAANNAFEWVADKDNENVVKLVSDAQGRFEITGLLAGTYYLEETKQPAGYALLTSRQKFEVTATSYSATGQGIEYTAGSGKDDATKVVNKKITLGHHHHHH

SEQ ID NO: 16 - protein, 309 aa, Streptococcus pneumoniae
MKKLGTLLVLFLSAIILVACASGKKDTTSGQKLKVVATNSIIADITKNIAGDKIDLHSIVPIGQDPHEYEPLPEDVKKTSEADLIFYNGINLETGGNAWFTKLVENAKKTENKDYFAVSDGVDVIYLEGQNEKGKEDPHAWLNLENGIIFAKNIAKQLSAKDPNNKEFYEKNLKEYTDKLDKLDKESKDKFNKIPAEKKLIVTSEGAFKYFSKAYGVPSAYIWEINTEEEGTPEQIKTLVEKLRQTKVPSLFVESSVDDRPMKTVSQDTNIPIYAQIFTDSIAEQGKEGDSYYSMMKYNLDKIAEGLAK

SEQ ID NO: 17 - protein, 1801 aa, Streptococcus pneumoniae
MASAEQKTKTLTVHKLLMTDQELDAWNSDAITTAGYDGSQNFEQFKQLQGVPQGVTEISGVAFELQSYTGPQGKEQENLTNDAVWTAVNKGVTTETGVKFDTEVLQGTYRLVEVRKESTYVGPNGKVLTGMKAVPALIILPLVNQNGVVENAHVYPKNSEDKPTATKTFDTAAGFVDPGEKGLAIGTKVPYIVTTTIPKNSTLATAFWSDEMTEGLDYNGDVVVNYNGQPLDNSHYTLEAGHNGFILKLNEKGLEAINGKDAEATITLKYTATLNALAVADVPEANDVTFHYGNNPGHGNTPKPNKPKNGELTITKTWADAKDAPIAGVEVTFDLVNAQTGEVVKVPGHETGIVLNQTNNWTFTATGLDNNTEYKFVERTIKGYSADYQTITETGKIAVKNWKDENPEPINPEEPRVKTYGKKFVKVDQKDERLKEAQFVVKNEQGKYLALKSAAQQAVNEKAAAEAKQALDAAIAAYTNAADKNAAQAVVDAAQKTYNDNYRAARFGYVEVERKEDALVLTSNTDGQFQISGLAAGSYTLEETKAPEGFAKLGDVKFEVGAGSWNQGDFNYLKDVQKNDATKVVNKKITGSGSGGGGAATVFAAGTTTTSVTVHKLLATDGDMDKIANELETGNYAGNKVGVLPANAKEIAGVMFVWTNTNNEIIDENGQTLGVNIDPQTFKLSGAMPATAMKKLTEAEGAKFNTANLPAAKYKIYEIHSLSTYVGEDGATLTGSKAVPIEIELPLNDVVDAHVYPKNTEAKPKIDKDFKGKANPDTPRVDKDTPVNHQVGDVVEYEIVTKIPALANYATANWSDRMTEGLAFNKGTVKVTVDDVALEAGDYALTEVATGFDLKLTDAGLAKVNDQNAEKTVKITYSATLNDKAIVEVPESNDVTFNYGNNPDHGNTPKPNKPNENGDLTLTKTWVDATGAPIPAGAEATFDLVNAQTGKVVQTVTLTTDKNTVTVNGLDKNTEYKFVERSIKGYSADYQEITTAGEIAVKNWKDENPKPLDPTEPKVVTYGKKFVKVNDKDNRLAGAEFVIANADNAGQYLARKADKVSQEEKQLVVTTKDALDRAVAAYNALTAQQQTQQEKEKVDKAQAAYNAAVIAANNAFEWVADKDNENVVKLVSDAQGRFEITGLLAGTYYLEETKQPAGYALLTSRQKFEVTATSYSATGQGIEYTAGSGKDDATKVVNKKITLGGSGGGGAATVFAADNVSTAPDAVTKTLTIHKLLLSEDDLKTWDTNGPKGYDGTQSSLKDLTGVVAEEIPNVYFELQKYNLTDGKEKENLKDDSKWTTVHGGLTTKDGLKIETSTLKGVYRIREDRTKTTYVGPNGQVLTGSKAVPALVTLPLVNNNGTVIDAHVFPKNSYNKPVVDKRIADTLNYNDQNGLSIGTKIPYVVNTTIPSNATFATSFWSDEMTEGLTYNEDVTITLNNVAMDQADYEVTKGNNGFNLKLTEAGLAKINGKDADQKIQITYSATLNSLAVADIPESNDITYHYGNHQDHGNTPKPTKPNNGQITVTKTWDSQPAPEGVKATVQLVNAKTGEKVGAPVELSENNWTYTWSGLDNSIEYKVEEEYNGYSAEYTVESKGKLGVKNWKDNNPAPINPEEPRVKTYGKKFVKVDQKDTRLENAQFVVKKADSNKYIAFKSTAQQAADEKAAATAKQKLDAAVAAYTNAADKQAAQALVDQAQQEYNVAYKEAKFGYVEVAGKDEAMVLTSNTDGQFQISGLAAGTYKLEEIKAPEGFAKIDDVEFVVGAGSWNQGEFNYLKDVQKNDATKVVNKKITLGHHHHHH

SEQ ID NO: 18 - protein, 619 aa, Streptococcus pneumoniae
MNKKKMILTSLASVAILGAGFVASQPTVVRAEESPVASQSKAEKDYDAAKKDAKNAKKAVEDAQKALDDAKAAQKKYDEDQKKTEEKAALEKAASEEMDKAVAAVQQAYLAYQQATDKAAKDAADKMIDEAKKREEEAKTKFNTVRAMVVPEPEQLAETKKKSEEAKQKAPELTKKLEEAKAKLEEAEKKATEAKQKVDAEEVAPQAKIAELENQVHRLEQELKEIDESESEDYAKEGFRAPLQSKLDAKKAKLSKLEELSDKIDELDAEIAKLEDQLKAAEENNNVEDYFKEGLEKTIAAKKAELEKTEADLKKAVNEPEKPAPAPETPAPEAPAEQPKPAPAPQPAPAPKPEKPAEQPKPEKTDDQQAEEDYARRSEEEYNRLTQQQPPKAEKPAPAPKTGWKQENGMWYFYNTDGSMATGWLQNNGSWYYLNSNGAMATGWLQYNGSWYYLNANGAMATGWAKVNGSWYYLNANGAMATGWLQYNGSWYYLNANGAMATGWAKVNGSWYYLNANGAMATGWLQYNGSWYYLNANGAMATGWAKVNGSWYYLNANGAMATGWVKDGDTWYYLEASGAMKASQWFKVSDKWYYVNGLGALAVNTTVDGYKVNANGEWV

SEQ ID NO: 19 - protein, 1801 aa, Streptococcus pneumoniae
MASAATVFAADNVSTAPDAVTKTLTIHKLLLSEDDLKTWDTNGPKGYDGTQSSLKDLTGVVAEEIPNVYFELQKYNLTDGKEKENLKDDSKWTTVHGGLTTKDGLKIETSTLKGVYRIREDRTKTTYVGPNGQVLTGSKAVPALVTLPLVNNNGTVIDAHVFPKNSYNKPVVDKRIADTLNYNDQNGLSIGTKIPYVVNTTIPSNATFATSFWSDEMTEGLTYNEDVTITLNNVAMDQADYEVTKGNNGFNLKLTEAGLAKINGKDADQKIQITYSATLNSLAVADIPESNDITYHYGNHQDHGNTPKPTKPNNGQITVTKTWDSQPAPEGVKATVQLVNAKTGEKVGAPVELSENNWTYTWSGLDNSIEYKVEEEYNGYSAEYTVESKGKLGVKNWKDNNPAPINPEEPRVKTYGKKFVKVDQKDTRLENAQFVVKKADSNKYIAFKSTAQQAADEKAAATAKQKLDAAVAAYTNAADKQAAQALVDQAQQEYNVAYKEAKFGYVEVAGKDEAMVLTSNTDGQFQISGLAAGTYKLEEIKAPEGFAKIDDVEFVVGAGSWNQGEFNYLKDVQKNDATKVVNKKITGSGSGGGGAEQKTKTLTVHKLLMTDQELDAWNSDAITTAGYDGSQNFEQFKQLQGVPQGVTEISGVAFELQSYTGPQGKEQENLTNDAVWTAVNKGVTTETGVKFDTEVLQGTYRLVEVRKESTYVGPNGKVLTGMKAVPALIILPLVNQNGVVENAHVYPKNSEDKPTATKTFDTAAGFVDPGEKGLAIGTKVPYIVTTTIPKNSTLATAFWSDEMTEGLDYNGDVVVNYNGQPLDNSHYTLEAGHNGFILKLNEKGLEAINGKDAEATITLKYTATLNALAVADVPEANDVTFHYGNNPGHGNTPKPNKPKNGELTITKTWADAKDAPIAGVEVTFDLVNAQTGEVVKVPGHETGIVLNQTNNWTFTATGLDNNTEYKFVERTIKGYSADYQTITETGKIAVKNWKDENPEPINPEEPRVKTYGKKFVKVDQKDERLKEAQFVVKNEQGKYLALKSAAQQAVNEKAAAEAKQALDAAIAAYTNAADKNAAQAVVDAAQKTYNDNYRAARFGYVEVERKEDALVLTSNTDGQFQISGLAAGSYTLEETKAPEGFAKLGDVKFEVGAGSWNQGDFNYLKDVQKNDATKVVNKKITLGGSGGGGAATVFAAGTTTTSVTVHKLLATDGDMDKIANELETGNYAGNKVGVLPANAKEIAGVMFVWTNTNNEIIDENGQTLGVNIDPQTFKLSGAMPATAMKKLTEAEGAKFNTANLPAAKYKIYEIHSLSTYVGEDGATLTGSKAVPIEIELPLNDVVDAHVYPKNTEAKPKIDKDFKGKANPDTPRVDKDTPVNHQVGDVVEYEIVTKIPALANYATANWSDRMTEGLAFNKGTVKVTVDDVALEAGDYALTEVATGFDLKLTDAGLAKVNDQNAEKTVKITYSATLNDKAIVEVPESNDVTFNYGNNPDHGNTPKPNKPNENGDLTLTKTWVDATGAPIPAGAEATFDLVNAQTGKVVQTVTLTTDKNTVTVNGLDKNTEYKFVERSIKGYSADYQEITTAGEIAVKNWKDENPKPLDPTEPKVVTYGKKFVKVNDKDNRLAGAEFVIANADNAGQYLARKADKVSQEEKQLVVTTKDALDRAVAAYNALTAQQQTQQEKEKVDKAQAAYNAAVIAANNAFEWVADKDNENVVKLVSDAQGRFEITGLLAGTYYLEETKQPAGYALLTSRQKFEVTATSYSATGQGIEYTAGSGKDDATKVVNKKITLGHHHHHH

SEQ ID NO: 20 - protein, 612 aa, Streptococcus pneumoniae
MFAFKKRRKVHYSIRKFSIGVASVAVASLFMGSVVHATEKEVTTQVATSSNKANKSQTEHMKAAKQVDEYIEKMLSEIQLDRRKHTQNVGLLTKLGAIKTEYLRGLSVSKEKSTAELPSEIKEKLTAAFEQFKKDTLKSGKKVAEAQKKAKDQKEAKQEIEALIVKHKGREIDLDRKKAKAAVTEHLKKLLNDIEKNLKKEQHTHTVELIKNLKDIEKTYLHKLDESTQKAQLQKLIAESQSKLDEAFSKFKNGLSSSSNSGSSTKPETPQPETPKPEVKPELETPKPEVKPEPETPKPEVKPEPETPKPEVKPELETPKPEVKPEPETPKPEVKPEPETPKPEVKPEPETPKPEVKPELETPKPEVKPELETPKPEVKPEPETPKPEVKPELETPKPEVKPEPETPKPEVKPELETPKPEVKPEPETPKPEVKPELETPKPEVKPEPETPKPEVKPEPETPKPEVKPEPETPKPEVKPELETPKQKVKPEPETPKPEVKPEPETPKPEVKPELETPKPEVKPELEIPKPEVKPDNSKPQADDKKPSTPNNLSKDKQSSNQASTNENKKQGPATNKPKKSLPSTGSISNLALEIAGLLTLAGATILAKKRMK

SEQ ID NO: 21 - protein, 1801 aa, Streptococcus pneumoniae
MASAATVFAADNVSTAPDAVTKTLTIHKLLLSEDDLKTWDTNGPKGYDGTQSSLKDLTGVVAEEIPNVYFELQKYNLTDGKEKENLKDDSKWTTVHGGLTTKDGLKIETSTLKGVYRIREDRTKTTYVGPNGQVLTGSKAVPALVTLPLVNNNGTVIDAHVFPKNSYNKPVVDKRIADTLNYNDQNGLSIGTKIPYVVNTTIPSNATFATSFWSDEMTEGLTYNEDVTITLNNVAMDQADYEVTKGNNGFNLKLTEAGLAKINGKDADQKIQITYSATLNSLAVADIPESNDITYHYGNHQDHGNTPKPTKPNNGQITVTKTWDSQPAPEGVKATVQLVNAKTGEKVGAPVELSENNWTYTWSGLDNSIEYKVEEEYNGYSAEYTVESKGKLGVKNWKDNNPAPINPEEPRVKTYGKKFVKVDQKDTRLENAQFVVKKADSNKYIAFKSTAQQAADEKAAATAKQKLDAAVAAYTNAADKQAAQALVDQAQQEYNVAYKEAKFGYVEVAGKDEAMVLTSNTDGQFQISGLAAGTYKLEEIKAPEGFAKIDDVEFVVGAGSWNQGEFNYLKDVQKNDATKVVNKKITGSGSGGGGAATVFAAGTTTTSVTVHKLLATDGDMDKIANELETGNYAGNKVGVLPANAKEIAGVMFVWTNTNNEIIDENGQTLGVNIDPQTFKLSGAMPATAMKKLTEAEGAKFNTANLPAAKYKIYEIHSLSTYVGEDGATLTGSKAVPIEIELPLNDVVDAHVYPKNTEAKPKIDKDFKGKANPDTPRVDKDTPVNHQVGDVVEYEIVTKIPALANYATANWSDRMTEGLAFNKGTVKVTVDDVALEAGDYALTEVATGFDLKLTDAGLAKVNDQNAEKTVKITYSATLNDKAIVEVPESNDVTFNYGNNPDHGNTPKPNKPNENGDLTLTKTWVDATGAPIPAGAEATFDLVNAQTGKVVQTVTLTTDKNTVTVNGLDKNTEYKFVERSIKGYSADYQEITTAGEIAVKNWKDENPKPLDPTEPKVVTYGKKFVKVNDKDNRLAGAEFVIANADNAGQYLARKADKVSQEEKQLVVTTKDALDRAVAAYNALTAQQQTQQEKEKVDKAQAAYNAAVIAANNAFEWVADKDNENVVKLVSDAQGRFEITGLLAGTYYLEETKQPAGYALLTSRQKFEVTATSYSATGQGIEYTAGSGKDDATKVVNKKITLGGSGGGGAEQKTKTLTVHKLLMTDQELDAWNSDAITTAGYDGSQNFEQFKQLQGVPQGVTEISGVAFELQSYTGPQGKEQENLTNDAVWTAVNKGVTTETGVKFDTEVLQGTYRLVEVRKESTYVGPNGKVLTGMKAVPALIILPLVNQNGVVENAHVYPKNSEDKPTATKTFDTAAGFVDPGEKGLAIGTKVPYIVTTTIPKNSTLATAFWSDEMTEGLDYNGDVVVNYNGQPLDNSHYTLEAGHNGFILKLNEKGLEAINGKDAEATITLKYTATLNALAVADVPEANDVTFHYGNNPGHGNTPKPNKPKNGELTITKTWADAKDAPIAGVEVTFDLVNAQTGEVVKVPGHETGIVLNQTNNWTFTATGLDNNTEYKFVERTIKGYSADYQTITETGKIAVKNWKDENPEPINPEEPRVKTYGKKFVKVDQKDERLKEAQFVVKNEQGKYLALKSAAQQAVNEKAAAEAKQALDAAIAAYTNAADKNAAQAVVDAAQKTYNDNYRAARFGYVEVERKEDALVLTSNTDGQFQISGLAAGSYTLEETKAPEGFAKLGDVKFEVGAGSWNQGDFNYLKDVQKNDATKVVNKKITLGHHHHHH

SEQ ID NO: 22 - protein, 279 aa, Streptococcus pneumoniae
MAVMAYPLVSRLYYRVESNQQIADFDKEKATLDEADIDERMKLAQAFNDSLNNVVSGDPWSEEMKKKGRAEYARMLEIHERMGHVEIPVIDVDLPVYAGTAEEVLQQGAGHLEGTSLPIGGNSTHAVITAHTGLPTAKMFTDLTKLKVGDKFYVHNIKEVMAYQVDQVKVIEPTNFDDLLIVPGHDYVTLLTCTPYMINTHRLLVRGHRIPYVAEVEEEFIAANKLSHLYRYLFYVAVGLIVILLWIIRRLRKKKKQPEKALKALKAARKEVKVEDGQQ

SEQ ID NO: 23 - protein, 1312 aa, Streptococcus pneumoniae
MKHEKQQRFSIRKYAVGAASVLIGFAFQAQTVAADGVTPTTTENQPTIHTVSDSPQSSENRTEETPKAELQPEAPKTVETETPATDKVASLPKTEEKPQEEVSSTPSDKAEVVTPTSAEKETANKKEEEASPKKEEAKEVDSKESNTDKTDKDKPAKKDEAKAEADKPETETGKERAATVNEKLAKKKIVSIDAGRKYFSPEQLKEIIDKAKHYGYTDLHLLVGNDGLRFMLDDMSITANGKTYASDDVKRAIEKGTNDYYNDPNGNHLTESQMTDLINYAKDKGIGLIPTVNSPGHMDAILNAMKELGIQNPNFSYFGKKSARTVDLDNEQAVAFTKALIDKYAAYFAKKTEIFNIGLDEYANDATDAKGWSVLQADKYYPNEGYPVKGYEKFIAYANDLARIVKSHGLKPMAFNDGIYYNSDTSFGSFDKDIIVSMWTGGWGGYDVASSKLLAEKGHQILNTNDAWCYVLGRNADGQGWYNLDQGLNGIKNTPITSVPKTEGADIPIIGGMVAAWADTPSARYSPSHLFKLMRHFANANAEYFAADYESAEQALNEVPKDLNRYTAESVAAVKEAEKAIRSLDSNLSRAQQDTIDQAIAKLQETVNNLTLTPEAQKEEEAKREVEKLAKNKVISIDAGRKYFTLDQLKRIVDKASELGYSDVHLLLGNDGLRFLLNDMTITANGKTYASDDVKKAIIEGTKAYYDDPNGTALTQAEVTELIEYAKSKDIGLIPAINSPGHMDAMLVAMEKLGIKNPQAHFDKVSKTTMDLKNEEAMNFVKALIGKYMDFFAGKTKIFNFGTDEYANDATSAQGWYYLKWYQLYGKFAEYANTLAAMAKERGLQPMAFNDGFYYEDKDDVQFDKDVLISYWSKGWWGYNLASPQYLASKGYKFLNTNGDWYYVIGNHKQDEAYPLSKAVENSGKVPFNQLASTKYPEVDLPTVGSMLSIWADRPSAEYKEEEIFELMTAFADHNKDYFRANYNALREELAKIPTNLEGYSKESLEALDAAKTALNYNLNRNKQAELDTLVANLKAALQGLKPAATHSGSLDENEVAANVETRPELITRTEEIPFEVIKKENPNLPAGQENIITAGVKGERTHYISVLTENGKTTETVLDSQVTKEVINQVVEVGSPVTHKGDESGLAPTTEVKPRLDIQEEEIPFTTVTRENPLLLKGKTQVITKGVNGHRSNFYSVSTSADGKEVKTLVNSVVAQEAVTQIVEVGTMVTHVGDENGQAAIAEEKPKLEIPSQPAPSTAPAEESKALPQDPAPVVTEKKLPETGTHDSAELVVAGLMSTLAAYGLTKRKED

SEQ ID NO: 24 - protein, 1062 aa, Streptococcus pneumoniae
LHLLVGNDGLRFMLDDMSITANGKTYASDDVKRAIEKGTNDYYNDPNGNHLTESQMTDLINYAKDKGIGLIPTVNSPGHMDAILNAMKELGIQNPNFSYFGKKSARTVDLDNEQAVAFTKALIDKYAAYFAKKTEIFNIGLDEYANDATDAKGWSVLQADKYYPNEGYPVKGYEKFIAYANDLARIVKSHGLKPMAFNDGIYYNSDTSFGSFDKDIIVSMWTGGWGGYDVASSKLLAEKGHQILNTNDAWCYVLGRNADGQGWYNLDQGLNGIKNTPITSVPKTEGADIPIIGGMVAAWADTPSARYSPSHLFKLMRHFANANAEYFAADYESAEQALNEVPKDLNRYTAESVAAVKEAEKAIRSLDSNLSRAQQDTIDQAIAKLQETVNNLTLTPEAQKEEEAKREVEKLAKNKVISIDAGRKYFTLDQLKRIVDKASELGYSDVHLLLGNDGLRFLLNDMTITANGKTYASDDVKKAIIEGTKAYYDDPNGTALTQAEVTELIEYAKSKDIGLIPAINSPGHMDAMLVAMEKLGIKNPQAHFDKVSKTTMDLKNEEAMNFVKALIGKYMDFFAGKTKIFNFGTDEYANDATSAQGWYYLKWYQLYGKFAEYANTLAAMAKERGLQPMAFNDGFYYEDKDDVQFDKDVLISYWSKGWWGYNLASPQYLASKGYKFLNTNGDWYYVIGNHKQDEAYPLSKAVENSGKVPFNQLASTKYPEVDLPTVGSMLSIWADRPSAEYKEEEIFELMTAFADHNKDYFRANYNALREELAKIPTNLEGYSKESLEALDAAKTALNYNLNRNKQAELDTLVANLKAALQGLKPAATHSGSLDENEVAANVETRPELITRTEEIPFEVIKKENPNLPAGQENIITAGVKGERTHYISVLTENGKTTETVLDSQVTKEVINQVVEVGSPVTHKGDESGLAPTTEVKPRLDIQEEEIPFTTVTRENPLLLKGKTQVITKGVNGHRSNFYSVSTSADGKEVKTLVNSVVAQEAVTQIVEVGTMVTHVGDENGQAAIAEEKPKLEIPSQPAPSTAPAEESKALPQDPAPVVTEKK

SEQ ID NO: 25 - protein, 2228 aa, Streptococcus pneumoniae
MGKGHWNRKRVYSIRKFAVGACSVMIGTCAVLLGGNIAGESVVYADETLITHTAEKPKEEKMIVEEKADKALETKNVVERTEQSEPSSTEAIASEKKEDEAVTPKEEKVSAKPEEKAPRIESQASSQEKPLKEDAKAVTNEEVNQMIENRKVDFNQNWYFKLNANSKEAIKPDADVSTWKKLDLPYDWSIFNDFDHESPAQNEGGQLNGGEAWYRKTFKLDEKDLKKNVRLTFDGVYMDSQVYVNGQLVGHYPNGYNQFSYDITKYLYKDGRENVIAVHAVNKQPSSRWYSGSGIYRDVTLQVTDKVHVEKNGTTILTPKLEEQQHGKVETHVTSKIVNTDDKDHELVAEYQIVERGGHAVTGLVRTASRTLKAHESTSLDAILEVERPKLWTVLNDKPALYELITRVYRDGQLVDAKKDLFGYRYYHWTPNEGFSLNGERIKFHGVSLHHDHGALGAEENYKAEYRRLKQMKEMGVNSIRTTHNPASEQTLQIAAELGLLVQEEAFDTWYGGKKPYDYGRFFEKDATHPEARKGEKWSDFDLRTMVERGKNNPAIFMWSIGNEIGEANGDAHSLATVKRLVKVIKDVDKTRYVTMGADKFRFGNGSGGHEKIADELDAVGFNYSEDNYKALRAKHPKWLIYGSETSSATRTRGSYYRPERELKHSNGPERNYEQSDYGNDRVGWGKTATASWTFDRDNAGYAGQFIWTGTDYIGEPTPWHNQNQTPVKSSYFGIVDTAGIPKHDFYLYQSQWVSVKKKPMVHLLPHWNWENKELASKVADSEGKIPVRAYSNASSVELFLNGKSLGLKTFNKKQTSDGRTYQEGANANELYLEWKVAYQPGTLEAIARDESGKEIARDKITTAGKPAAVRLIKEDHAIAADGKDLTYIYYEIVDSQGNVVPTANNLVRFQLHGQGQLVGVDNGEQASRERYKAQADGSWIRKAFNGKGVAIVKSTEQAGKFTLTAHSDLLKSNQVTVFTGKKEGQEKTVLGTEVPKVQTIIGEAPEMPTTVPFVYSDGSRAERPVTWSLVDVSKPGIVTVKGMADGREVEARVEVIALKSELPVVKRIAPNTNLNSVDKSVSYVLTDGSVQEYEVDKWEIAEEDKAKLAIPGSRIQATGYLEGQPIHATLVVEEGNPAAPVVPTVTVGGEAVTGLTSRQPMQYRTLSYGAQLPEVTASAENADVTVLQASAANGMRASIFIQPKDGGPLQTYAIQFLEEAPKIAHLSLQVEKADSLKEDQTVKLSVRAHYQDGTQAVLPADKVTFSTSGEGEVAIRKGMLELHKPGAVTLNAEYEGAKGQVELTIQANTEKKIAQSIRPVNVVTDLHQEPSLPATVTVEYDKGFPKTHKVTWQAIPKEKLDSYQIFEVLGKVEGIDLEARAKVSVEGIVSVEEVSVTTPIAEAPQLPESVRTYDSNGHVSSAKVAWDAIRPEQYAKEGVFTVNGRLEGTQLTTKLHVRVSAQTEQGANISDQWTGSELPLAFASDSNPSDPVSNVNDKLISYNNQPANRWTNWNRSNPEASVGVLFGDSGILSKRSVDNLSVGFHEDHGVGAPKSYVIEYYVGKTVPTAPKNPSFVGNEDHVFNDSANWKPVTNLKAPAQLKAGEMNHFSFDKVETYAIRIRMVKADNKRGTSITEVQIFAKQVAAAKQGQTRIQVDGKDLANFNPDLTDYYLESVDGKVPAVTANVSNNGLATVVPSVREGEPVRVIAKAENGDILGEYRLHFTKDKNLLSHKPVAAVKQARLLQVGQALELPTKVPVYFTGKDGYETKDLTVEWEEVPAENLTKAGQFTVRGRVLGSNLVAEVTVRVTDKLGETLSDNPNYDENSNQAFASATNDIDKNSHDRVDYLNDGDHSENRRWTNWSPTPSSNPEVSAGVIFRENGKIVERTVAQAKLHFFADSGTDAPSKLVLERYVGPGFEVPTYYSNYQAYESGHPFNNPENWEAVPYRADKDIAAGDEINVTFKAVKAKVMRWRMERKADKSGVAMIEMTFLAPSELPQESTQSKILVDGKELADFAENRQDYQITYKGQRPKVSVEENNQVASTVVDSGEDSLPVLVRLVSESGKQVKEYRIQLTKEKPVSAVQEDLPKLEFVEKDLAYKTVEKKDSTLYLGETRVEQEGKVGKERIFTVINPDGSKEEKLREVVEVPTDRIVLVGTKPVAQEAKKPQVSEKADTKPIDSSEADQTNKAQLPNTGSAASQAAVAAGLALLGLSAGLVVTKGKKED

SEQ ID NO: 26 - protein, 247 aa, Streptococcus pneumoniae
MSQKNNKKKNKRKNLLTNILAGFLILLSLALIFNTQIRNIFIVWNTNKYQVSQVSKEKLEENQDTEGNFDFDSVKAISSEAVLTSQWDAQKLPVIGGIAIPELEMNLPIFKGLDNVNLFYGAGTMKREQVMGEGNYSLASHHIFGVDNANKMLFSPLDNAKNGMKIYLTDKNKVYTYEIREVKRVTPDRVDEVDDRDGVNEITLVTCEDLAATERIIVKGDLKETKDYSQTSDEILTAFNQPYKQFY

SEQ ID NO: 27 - protein, 1062 aa, Streptococcus pneumoniae
LHLLVGNDGLRFMLDDMSITANGKTYASDDVKRAIEKGTNDYYNDPNGNHLTESQMTDLINYAKDKGIGLIPTVNSPGHMDAILNAMKELGIQNPNFSYFGKKSARTVDLDNEQAVAFTKALIDKYAAYFAKKTEIFNIGLDEYANDATDAKGWSVLQADKYYPNEGYPVKGYEKFIAYANDLARIVKSHGLKPMAFNDGIYYNSDTSFGSFDKDIIVSMWTGGWGGYDVASSKLLAEKGHQILNTNDAWYYVLGRNADGQGWYNLDQGLNGIKNTPITSVPKTEGADIPIIGGMVAAWADTPSARYSPSRLFKLMRHFANANAEYFAADYESAEQALNEVPKDLNRYTAESVTAVKEAEKAIRSLDSNLSRAQQDTIDQAIAKLQETVNNLTLTPEAQKEEEAKREVEKLAKNKVISIDAGRKYFTLNQLKRIVDKASELGYSDVHLLLGNDGLRFLLDDMTITANGKTYASDDVKKAIIEGTKAYYDDPNGTALTQAEVTELIEYAKSKDIGLIPAINSPGHMDAMLVAMEKLGIKNPQAHFDKVSKTTMDLKNEEAMNFVKALIGKYMDFFAGKTKIFNFGTDEYANDATSAQGWYYLKWYQLYGKFAEYANTLAAMAKERGLQPMAFNDGFYYEDKDDVQFDKDVLISYWSKGWWGYNLASPQYLASKGYKFLNTNGDWYYILGQKPEDGGGFLKKAIENTGKTPFNQLASTKYPEVDLPTVGSMLSIWADRPSAEYKEEEIFELMTAFADHNKDYFRANYNALREELAKIPTNLEGYSKESLEALDAAKTALNYNLNRNKQAELDTLVANLKAALQGLKPAVTHSGSLDENEVAANVETRPELITRTEEIPFEVIKKENPNLPAGQENIITAGVKGERTHYISVLTENGKTTETVLDSQVTKEVINQVVEVGAPVTHKGDESGLAPTTEVKPRLDIQEEEIPFTTVTCENPLLLKGKTQVITKGVNGHRSNFYSVSTSADGKEVKTLVNSVVAQEAVTQIVEVGTMVTHVGDENGQAAIAEEKPKLEIPSQPAPSTAPAEESKVLPQDPAPVVTEKK

SEQ ID NO: 28 - protein, 128 aa, Streptococcus pneumoniae
MEKDMNLKREQEFVSQYHFDARNFEWENENGAPETKVDVNFQLLQHDQENQVTSLIVILSFMIVFDKFVISGTISQVNHIDGRIVNEPNELNQEEVETLARPCLNMLNRLTYEVTEIALDLPGINLEF

SEQ ID NO: 29 - protein, 259 aa, Streptococcus pneumoniae
MKKNSLYIISSLFFACVLFVYATATNFQNSTSARQVKTETYTNTVTNVPIDIRYNSDKYFISGFASEVSVVLTGANRLSLASEMQESTRKFKVTADLTDAGVGTIEVPLSIEDLPNGLTAVATPQKITVKIGKKAQKDKVKIVPEIDPSQIDSRVQIENVMVSDKEVSITSDQETLDRIDKIIAVLPTSERITGNYSGSVPLQAIDRNGVVLPAVITPFDTIMKVTTKPVAPSSSTSNSSTSSSSETSSSTKATSSKTN

SEQ ID NO: 30 - protein, 721 aa, Streptococcus pneumoniae
MNLGEFWYNKINKNRGRRLMKKVRFIFLALLFFLASPEGAMASDGTWQGKQYLKEDGSQAANEWVFDTHYQSWFYIKADANYAENEWLKQGDDYFYLKSGGYMAKSEWVEDKGAFYYLDQDGKMKRNAWVGTSYVGATGAKVIEDWVYDSQYDAWFYIKADGQHAEKEWLQIKGKDYYFKSGGYLLTSQWINQAYVNASGAKVQQGWLFDKQYQSWFYIKENGNYADKEWIFENGHYYYLKSGGYMAANEWIWDKESWFYLKFDGKIAEKEWVYDSHSQAWYYFKSGGYMAANEWIWDKESWFYLKFDGKMAEKEWVYDSHSQAWYYFKSGGYMTANEWIWDKESWFYLKSDGKIAEKEWVYDSHSQAWYYFKSGGYMTANEWIWDKESWFYLKSDGKMAEKEWVYDSHSQAWYYFKSGGYMAKNETVDGYQLGSDGKWLGGKATNKNAAYYQVVPVTANVYDSDGEKLSYISQGSVVWLDKDRKSDDKRLAITISGLSGYMKTEDLQALDASKDFIPYYESDGHRFYHYVAQNASIPVASHLSDMEVGKKYYSADGLHFDGFKLENPFLFKDLTEATNYSAEELDKVFSLLNINNSLLENKGATFKEAEEHYHINALYLLAHSALESNWGRSKIAKDKNNFFGITAYDTTPYLSAKTFDDVDKGILGATKWIKENYIDRGRTFLGNKASGMNVEYASDPYWGEKIASVMMKINEKLGGKD

SEQ ID NO: 31 - protein, 501 aa, Streptococcus pneumoniae
MSVTFFIGEERLKIKIGLASICLLGLATSHVAANETEVAKTSQDTTTASSSSEQNQSSNKTQTSAEVQTNAAAYWDGDYYVKDDGSKAQSEWIFDNYYKAWFYINSDGRYSQNEWHGNYYLKSGGYMAQNEWIYDSNYKSWFYLKSDGAYAHQEWQLIGNKWYYFKKWGYMAKSQWQGSYFLNGQGAMIQNEWLYDPAYSAYFYLKSDGTYANQEWQKVGGKWYYFKKWGYMARNEWQGNYYLTGSGAMATDEVIMDGARYIFAASGELKEKKDLNVGWVHRDGKRYFFNNREEQVGTEHAKKIIDISEHNGRINDWKKVIDENKVDGVIVRLGYSGKEDKELAHNIKELNRLGIPYGVYLYTYAENETDAENDAKQTIELIKKYNMNLSYPIYYDVENWEYVNKSKRAPSDTDTWVKIINKYMDTMKQAGYQNVYVYSYRSLLQTRLKHPDILKHVNWVAAYTNALEWENPYYSGEKGWQYTSSEYMKGIQGRVDVSVWY

SEQ ID NO: 32 - protein, 471 aa, Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 33 - protein, 392 aa, Streptococcus pneumoniae
MKKKILASLLLSTVMVSQVAVLTTAHAETTDDKIAAQDNKISNLTAQQQEAQKQVDQIQEQVSAIQAEQSNLQAENDRLQAESKKLEGEITELSKNIVSRNQSLEKQARSAQTNGAVTSYINTIVNSKSITEAISRVAAMSEIVSANNKMLEQQKADKKAISEKQVANNDAINTVIANQQKLADDAQALTTKQAELKAAELSLAAEKATAEGEKASLLEQKAAAEAEARAAAVAEAAYKEKRASQQQSVLASANTNLTAQVQAVSESAAAPVRAKVRPTYSTNASSYPIGECTWGVKTLAPWAGDYWGNGAQWATSAAAAGFRTGSTPQVGAIACWNDGGYGHVAVVTAVESTTRIQVSESNYAGNRTIGNHRGWFNPTTTSEGFVTYIYAD

SEQ ID NO: 34 - protein, 167 aa, Streptococcus pneumoniae
MKSITKKIKATLAGVAALFAVFAPSFVSAQESSTYTVKEGDTLSEIAETHNTTVEKLAENNHIDNIHLIYVDQELVIDGPVAPVATPAPATYAAPAAQDETVSAPVAETPVVSETVVSTVSGSEAEAKEWIAQKESGGSYTATNGRYIGRYGSWTAAKNFWLNNGWY

SEQ ID NO: 35 - protein, 380 aa, Streptococcus pneumoniae
MKKRMLLASTVALSFAPVLATQAEEVLWTARSVEQIQNDLTKTDNKTSYTVQYGDTLSTIAEALGVDVTVLANLNKITNMDLIFPETVLTTTVNEAEEVTEVEIQTPQADSSEEVTTATADLTTNQVTVDDQTVQVADLSQPIAEAPKEVASSSEVTKTVIASEEVAPSTGTSVPEEQTAETSSAVAEEAPQETTPAEKQETQTSPQAASAVEATTTSSEAKEVASSNGATAAVSTYQPEETKIISTTYEAPAAPDYAGLAVAKSENAGLQPQTAAFKEEIANLFGITSFSGYRPGDSGDHGKGLAIDFMVPERSELGDKIAEYAIQNMASRGISYIIWKQRFYAPFDSKYGPANTWNPMPDRGSVTENHYDHVHVSMNG

SEQ ID NO: 36 - protein, 659 aa, Streptococcus pneumoniae
MKKNRVFATAGLVLLAAGVLAACSSSKSSDSSAPKAYGYVYTADPETLDYLISRKNSTTVVTSNGIDGLFTNDNYGNLAPAVAEDWEVSKDGLTYTYKIRKGVKWFTSDGEEYAEVTAKDFVNGLKHAADKKSEAMYLAENSVKGLADYLSGTSTDFSTVGVKAVDDYTLQYTLNQPEPFWNSKLTYSIFWPLNEEFETSKGSDFAKPTDPTSLLYNGPFLLKGLTAKSSVEFVKNEQYWDKENVHLDTINLAYYDGSDQESLERNFTSGAYSYARLYPTSSNYSKVAEEYKDNIYYTQSGSGIAGLGVNIDRQSYNYTSKTTDSEKVATKKALLNKDFRQALNFALDRSAYSAQINGKDGAALAVRNLFVKPDFVSAGEKTFGDLVAAQLPAYGDEWKGVNLADGQDGLFNADKAKAEFAKAKKALEADGVQFPIHLDVPVDQASKNYISRIQSFKQSVETVLGVENVVVDIQQMTSDEFLNITYYAANASSEDWDVSGGVSWGPDYQDPSTYLDILKTTSSETTKTYLGFDNPNSPSVVQVGLKEYDKLVDEAAKETSDLNVRYEKYAAAQAWLTDSSLFIPAMASSGAAPVLSRIVPFTGASAQTGSKGSDVYFKYLKLQDKAVTKEEYEKAREKWLKEKAESNEKAQKELASHVK

SEQ ID NO: 37 - protein, 313 aa, Streptococcus pneumoniae
MKKKLLAGAITLLSVATLAACSKGSEGADLISMKGDVITEHQFYEQVKNNPSAQQVLLNMTIQKVFEKQYGSELDDKEVDDTIAEEKKQYGENYQRVLSQAGMTLETRKAQIRTSKLVELAVKKVAEAELTDEAYKKAFDEYTPDVTAQIIRLNNEDKAKEVLEKAKAEGADFAQLAKDNSTDEKTKENGGEITFDSASTEVPEQVKKAAFALDVDGVSDVITATGTQAYSSQYYIVKLTKKTEKSSNIDDYKEKLKTVILTQKQNDSTFVQSIIGKELQAANIKVKDQAFQNIFTQYIGGGDSSSSSSTSNE

SEQ ID NO: 38 - protein, 1247 aa, Streptococcus pneumoniae
ADGVTPTTTENQPTIHTVSDSPQSSENRTEETPKAELQPEAPKTVETETPATDKVASLPKTEEKPQEEVSSTPSDKAEVVTPTSAEKETANKKEEEASPKKEEAKEVDSKESNTDKTDKDKPAKKDEAKAEADKPETETGKERAATVNEKLAKKKIVSIDAGRKYFSPEQLKEIIDKAKHYGYTDLHLLVGNDGLRFMLDDMSITANGKTYASDDVKRAIEKGTNDYYNDPNGNHLTESQMTDLINYAKDKGIGLIPTVNSPGHMDAILNAMKELGIQNPNFSYFGKKSARTVDLDNEQAVAFTKALIDKYAAYFAKKTEIFNIGLDEYANDATDAKGWSVLQADKYYPNEGYPVKGYEKFIAYANDLARIVKSHGLKPMAFNDGIYYNSDTSFGSFDKDIIVSMWTGGWGGYDVASSKLLAEKGHQILNTNDAWCYVLGRNADGQGWYNLDQGLNGIKNTPITSVPKTEGADIPIIGGMVAAWADTPSARYSPSHLFKLMRHFANANAEYFAADYESAEQALNEVPKDLNRYTAESVAAVKEAEKAIRSLDSNLSRAQQDTIDQAIAKLQETVNNLTLTPEAQKEEEAKREVEKLAKNKVISIDAGRKYFTLDQLKRIVDKASELGYSDVHLLLGNDGLRFLLNDMTITANGKTYASDDVKKAIIEGTKAYYDDPNGTALTQAEVTELIEYAKSKDIGLIPAINSPGHMDAMLVAMEKLGIKNPQAHFDKVSKTTMDLKNEEAMNFVKALIGKYMDFFAGKTKIFNFGTDEYANDATSAQGWYYLKWYQLYGKFAEYANTLAAMAKERGLQPMAFNDGFYYEDKDDVQFDKDVLISYWSKGWWGYNLASPQYLASKGYKFLNTNGDWYYVIGNHKQDEAYPLSKAVENSGKVPFNQLASTKYPEVDLPTVGSMLSIWADRPSAEYKEEEIFELMTAFADHNKDYFRANYNALREELAKIPTNLEGYSKESLEALDAAKTALNYNLNRNKQAELDTLVANLKAALQGLKPAATHSGSLDENEVAANVETRPELITRTEEIPFEVIKKENPNLPAGQENIITAGVKGERTHYISVLTENGKTTETVLDSQVTKEVINQVVEVGSPVTHKGDESGLAPTTEVKPRLDIQEEEIPFTTVTRENPLLLKGKTQVITKGVNGHRSNFYSVSTSADGKEVKTLVNSVVAQEAVTQIVEVGTMVTHVGDENGQAAIAEEKPKLEIPSQPAPSTAPAEESKALPQDPAPVVTEKK

SEQ ID NO: 39 - protein, 339 aa, Streptococcus pneumoniae
MVKKNDLFVDVSSHNGYDITGILEQMGTTNTIIKISESTTYLNPCLSAQVEQSNPIGFYHFARFGGDVAEAEREAQFFLDNVPMQVKYLVLDYEDDPSGDAQANTNACLRFMQMIADAGYKPIYYSYKPFTHDNVDYQQILAQFPNSLWIAGYGLNDGTANFEYFPSMDGIRWWQYSSNPFDKNIVLLDDEEDDKPKTAGTWKQDSKGWWFRRNNGSFPYNKWEKIGGVWYYFDSKGYCLTSEWLKDNEKWYYLKDNGAMATGWVLVGSEWYYMDDSGAMVTGWVKYKNNWYYMTNERGNMVSNEFIKSGKGWYFMNTNGELADNPSFTKEPDGLITVA

SEQ ID NO: 40 - protein, 628 aa, Streptococcus pneumoniae
GAEETTTNTIQQSQKEVQYQQRDTKNLVENGDFGQTEDGSSPWTGSKAQGWSAWVDQKNSSADASTRVIEAKDGAITISSPEKLRAAVHRMVPIEAKKKYKLRFKIKTDNKVGIAKVRIIEESGKDKRLWNSATTSGTKDWQTIEADYSPTLDVDKIKLELFYETGTGTVSFKDIELVEVADQPSEDSQTDKQLEEKIDLPIGKKHVFSLADYTYKVENPDVASVKNGILEPLKEGTTNVIVSKDGKEVKKIPLKILASVKDTYTDRLDDWNGIIAGNQYYDSKNEQMAKLNQELEGKVADSLSSISSQADRIYLWEKFSNYKTSANLTATYRKLEEMAKQVTNPSSRYYQDETVVRTVRDSMEWMHKHVYNSEKSIVGNWWDYEIGTPRAINNTLSLMKEYFSDEEIKKYTDVIEKFVPDPEHFRKTTDNPFKALGGNLVDMGRVKVIAGLLRKDDQEISSTIRSIEQVFKLVDQGEGFYQDGSYIDHTNVAYTGAYGNVLIDGLSQLLPVIQKTKNPIDKDKMQTMYHWIDKSFAPLLVNGELMDMSRGRSISRANSEGHVAAVEVLRGIHRIADMSEGETKQRLQSLVKTIVQSDSYYDVFKNLKTYKDISLMQSLLSDAGVASV

SEQ ID NO: 41 - protein, 204 aa, Streptococcus pneumoniae
MFKRIRRVLVLAVFLFAGYKAYRVHQDVKQVMTYQPMVREILSEQDTPANEELVLAMIYTETKGKEGDVMQSSESASGSTNTINDNASSIRQGIQTLTGNLYLAQKKGVDIWTAVQAYNFGPAYIDFIAQNGKENTLALAKQYSRETVAPLLGNRTGKTYSYIHPISIFHGAELYVNGGNYYYSRQVRLNLYIIKCFTLFSTSG

SEQ ID NO: 42 - protein, 2148 aa, Streptococcus pneumoniae
DETLITHTAEKPKEEKMIVEEKADKALETKNVVERTEQSEPSSTEAIASEKKEDEAVTPKEEKVSAKPEEKAPRIESQASSQEKPLKEDAKAVTNEEVNQMIENRKVDFNQNWYFKLNANSKEAIKPDADVSTWKKLDLPYDWSIFNDFDHESPAQNEGGQLNGGEAWYRKTFKLDEKDLKKNVRLTFDGVYMDSQVYVNGQLVGHYPNGYNQFSYDITKYLYKDGRENVIAVHAVNKQPSSRWYSGSGIYRDVTLQVTDKVHVEKNGTTILTPKLEEQQHGKVETHVTSKIVNTDDKDHELVAEYQIVERGGHAVTGLVRTASRTLKAHESTSLDAILEVERPKLWTVLNDKPALYELITRVYRDGQLVDAKKDLFGYRYYHWTPNEGFSLNGERIKFHGVSLHHDHGALGAEENYKAEYRRLKQMKEMGVNSIRTTHNPASEQTLQIAAELGLLVQEEAFDTWYGGKKPYDYGRFFEKDATHPEARKGEKWSDFDLRTMVERGKNNPAIFMWSIGNEIGEANGDAHSLATVKRLVKVIKDVDKTRYVTMGADKFRFGNGSGGHEKIADELDAVGFNYSEDNYKALRAKHPKWLIYGSETSSATRTRGSYYRPERELKHSNGPERNYEQSDYGNDRVGWGKTATASWTFDRDNAGYAGQFIWTGTDYIGEPTPWHNQNQTPVKSSYFGIVDTAGIPKHDFYLYQSQWVSVKKKPMVHLLPHWNWENKELASKVADSEGKIPVRAYSNASSVELFLNGKSLGLKTFNKKQTSDGRTYQEGANANELYLEWKVAYQPGTLEAIARDESGKEIARDKITTAGKPAAVRLIKEDHAIAADGKDLTYIYYEIVDSQGNVVPTANNLVRFQLHGQGQLVGVDNGEQASRERYKAQADGSWIRKAFNGKGVAIVKSTEQAGKFTLTAHSDLLKSNQVTVFTGKKEGQEKTVLGTEVPKVQTIIGEAPEMPTTVPFVYSDGSRAERPVTWSLVDVSKPGIVTVKGMADGREVEARVEVIALKSELPVVKRIAPNTNLNSVDKSVSYVLTDGSVQEYEVDKWEIAEEDKAKLAIPGSRIQATGYLEGQPIHATLVVEEGNPAAPVVPTVTVGGEAVTGLTSRQPMQYRTLSYGAQLPEVTASAENADVTVLQASAANGMRASIFIQPKDGGPLQTYAIQFLEEAPKIAHLSLQVEKADSLKEDQTVKLSVRAHYQDGTQAVLPADKVTFSTSGEGEVAIRKGMLELHKPGAVTLNAEYEGAKGQVELTIQANTEKKIAQSIRPVNVVTDLHQEPSLPATVTVEYDKGFPKTHKVTWQAIPKEKLDSYQIFEVLGKVEGIDLEARAKVSVEGIVSVEEVSVTTPIAEAPQLPESVRTYDSNGHVSSAKVAWDAIRPEQYAKEGVFTVNGRLEGTQLTTKLHVRVSAQTEQGANISDQWTGSELPLAFASDSNPSDPVSNVNDKLISYNNQPANRWTNWNRSNPEASVGVLFGDSGILSKRSVDNLSVGFHEDHGVGAPKSYVIEYYVGKTVPTAPKNPSFVGNEDHVFNDSANWKPVTNLKAPAQLKAGEMNHFSFDKVETYAIRIRMVKADNKRGTSITEVQIFAKQVAAAKQGQTRIQVDGKDLANFNPDLTDYYLESVDGKVPAVTANVSNNGLATVVPSVREGEPVRVIAKAENGDILGEYRLHFTKDKNLLSHKPVAAVKQARLLQVGQALELPTKVPVYFTGKDGYETKDLTVEWEEVPAENLTKAGQFTVRGRVLGSNLVAEVTVRVTDKLGETLSDNPNYDENSNQAFASATNDIDKNSHDRVDYLNDGDHSENRRWTNWSPTPSSNPEVSAGVIFRENGKIVERTVAQAKLHFFADSGTDAPSKLVLERYVGPGFEVPTYYSNYQAYESGHPFNNPENWEAVPYRADKDIAAGDEINVTFKAVKAKVMRWRMERKADKSGVAMIEMTFLAPSELPQESTQSKILVDGKELADFAENRQDYQITYKGQRPKVSVEENNQVASTVVDSGEDSLPVLVRLVSESGKQVKEYRIQLTKEKPVSAVQEDLPKLEFVEKDLAYKTVEKKDSTLYLGETRVEQEGKVGKERIFTVINPDGSKEEKLREVVEVPTDRIVLVGTKPVAQEAKKPQVSEKADTKPIDSSEADQTNKAQ

SEQ ID NO: 43 - protein, 1063 aa, Streptococcus pneumoniae
DETLITHTAEKPKEEKMIVEEKADKALETKNVVERTEQSEPSSTEAIASEKKEDEAVTPKEEKVSAKPEEKAPRIESQASSQEKPLKEDAKAVTNEEVNQMIENRKVDFNQNWYFKLNANSKEAIKPDADVSTWKKLDLPYDWSIFNDFDHESPAQNEGGQLNGGEAWYRKTFKLDEKDLKKNVRLTFDGVYMDSQVYVNGQLVGHYPNGYNQFSYDITKYLYKDGRENVIAVHAVNKQPSSRWYSGSGIYRDVTLQVTDKVHVEKNGTTILTPKLEEQQHGKVETHVTSKIVNTDDKDHELVAEYQIVERGGHAVTGLVRTASRTLKAHESTSLDAILEVERPKLWTVLNDKPALYELITRVYRDGQLVDAKKDLFGYRYYHWTPNEGFSLNGERIKFHGVSLHHDHGALGAEENYKAEYRRLKQMKEMGVNSIRTTHNPASEQTLQIAAELGLLVQEEAFDTWYGGKKPYDYGRFFEKDATHPEARKGEKWSDFDLRTMVERGKNNPAIFMWSIGNEIGEANGDAHSLATVKRLVKVIKDVDKTRYVTMGADKFRFGNGSGGHEKIADELDAVGFNYSEDNYKALRAKHPKWLIYGSETSSATRTRGSYYRPERELKHSNGPERNYEQSDYGNDRVGWGKTATASWTFDRDNAGYAGQFIWTGTDYIGEPTPWHNQNQTPVKSSYFGIVDTAGIPKHDFYLYQSQWVSVKKKPMVHLLPHWNWENKELASKVADSEGKIPVRAYSNASSVELFLNGKSLGLKTFNKKQTSDGRTYQEGANANELYLEWKVAYQPGTLEAIARDESGKEIARDKITTAGKPAAVRLIKEDHAIAADGKDLTYIYYEIVDSQGNVVPTANNLVRFQLHGQGQLVGVDNGEQASRERYKAQADGSWIRKAFNGKGVAIVKSTEQAGKFTLTAHSDLLKSNQVTVFTGKKEGQEKTVLGTEVPKVQTIIGEAPEMPTTVPFVYSDGSRAERPVTWSLVDVSKPGIVTVKGMADGREVEARVEVIALKSELPVVKRIAPNTNLNSVDKSVSYVLTDGSVQEYEVDKWEIAEEDKAK

SEQ ID NO: 44 - protein, 1093 aa, Streptococcus pneumoniae
IAEEDKAKLAIPGSRIQATGYLEGQPIHATLVVEEGNPAAPVVPTVTVGGEAVTGLTSRQPMQYRTLSYGAQLPEVTASAENADVTVLQASAANGMRASIFIQPKDGGPLQTYAIQFLEEAPKIAHLSLQVEKADSLKEDQTVKLSVRAHYQDGTQAVLPADKVTFSTSGEGEVAIRKGMLELHKPGAVTLNAEYEGAKGQVELTIQANTEKKIAQSIRPVNVVTDLHQEPSLPATVTVEYDKGFPKTHKVTWQAIPKEKLDSYQIFEVLGKVEGIDLEARAKVSVEGIVSVEEVSVTTPIAEAPQLPESVRTYDSNGHVSSAKVAWDAIRPEQYAKEGVFTVNGRLEGTQLTTKLHVRVSAQTEQGANISDQWTGSELPLAFASDSNPSDPVSNVNDKLISYNNQPANRWTNWNRSNPEASVGVLFGDSGILSKRSVDNLSVGFHEDHGVGAPKSYVIEYYVGKTVPTAPKNPSFVGNEDHVFNDSANWKPVTNLKAPAQLKAGEMNHFSFDKVETYAIRIRMVKADNKRGTSITEVQIFAKQVAAAKQGQTRIQVDGKDLANFNPDLTDYYLESVDGKVPAVTANVSNNGLATVVPSVREGEPVRVIAKAENGDILGEYRLHFTKDKNLLSHKPVAAVKQARLLQVGQALELPTKVPVYFTGKDGYETKDLTVEWEEVPAENLTKAGQFTVRGRVLGSNLVAEVTVRVTDKLGETLSDNPNYDENSNQAFASATNDIDKNSHDRVDYLNDGDHSENRRWTNWSPTPSSNPEVSAGVIFRENGKIVERTVAQAKLHFFADSGTDAPSKLVLERYVGPGFEVPTYYSNYQAYESGHPFNNPENWEAVPYRADKDIAAGDEINVTFKAVKAKVMRWRMERKADKSGVAMIEMTFLAPSELPQESTQSKILVDGKELADFAENRQDYQITYKGQRPKVSVEENNQVASTVVDSGEDSLPVLVRLVSESGKQVKEYRIQLTKEKPVSAVQEDLPKLEFVEKDLAYKTVEKKDSTLYLGETRVEQEGKVGKERIFTVINPDGSKEEKLREVVEVPTDRIVLVGTKPVAQEAKKPQVSEKADTKPIDSSEADQTNKAQ

SEQ ID NO: 45 - protein, 2233 aa, Streptococcus pneumoniae
MGKGHWNRKRVYSIRKFAVGACSVMIGTCAVLLGGNIAGESVVYADETLITHTAEKPKEEKMIVEEKADKALETKNIVERTEQSEPSSTEAIASEKKEDEAVTPKEEKVSAKPEEKAPRIESQASNQEKPLKEDAKAVTNEEVNQMIEDRKVDFNQNWYFKLNANSKEAIKPDADVSTWKKLDLPYDWSIFNDFDHESPAQNEGGQLNGGEAWYRKTFKLDEKDLKKNVRLTFDGVYMDSQVYVNGQLVGHYPNGYNQFSYDITKYLQKDGRENVIAVHAVNKQPSSRWYSGSGIYRDVTLQVTDKVHVEKNGTTILTPKLEEQQHGKVETHVTSKIVNTDDKDHELVAEYQIVERGGHAVTGLVRTASRTLKAHESTSLDAILEVERPKLWTVLNDKPALYELITRVYRDGQLVDAKKDLFGYRYYHWTPNEGFSLNGERIKFHGVSLHHDHGALGAEENYKAEYRRLKQMKEMGVNSIRTTHNPASEQTLQIAAELGLLVQEEAFDTWYGGKKPYDYGRFFEKDATHPEARKGEKWSDFDLRTMVERGKNNPAIFMWSIGNEIGEANGDAHSLATVKRLVKVIKDVDKTRYVTMGADKFRFGNGSGGHEKIADELDAVGFNYSEDNYKALRAKHPKWLIYGSETSSATRTRGSYYRPERELKHSNGPERNYEQSDYGNDRVGWGKTATASWTFDRDNAGYAGQFIWTGTDYIGEPTPWHNQNQTPVKSSYFGIVDTAGIPKHDFYLYQSQWVSVKKKPMVHLLPHWNWENKELASKVADSEGKIPVRAYSNASSVELFLNGKSLGLKTFNKKQTSDGRTYQEGANANELYLEWKVAYQPGTLEAIARDESGKEIARDKITTAGKPAAVRLIKEDHAIAADGKDLTYIYYEIVDSQGNVVPTANNLVRFQLHGQGQLVGVDNGEQASRERYKAQADGSWIRKAFNGKGVAIVKSTEQAGKFTLTAHSDLLKSNQVTVFTGKKEGQEKTVLGTEVPKVQTIIGEAPEMPTTVPFVYSDGSRAERPVTWSSVDVSKPGIVTVKGMADGREVEARVEVIALKSELPVVKRIAPNTDLNSVDKSVSYVLIDGSVEEYEVDKWEIAEEDKAKLAIPGSRIQATGYLEGQPIHATLVVEEGNPAAPAVPTVTVGGEAVTGLTSQKPMQYRTLAYGAKLPEVTASAKNAAVTVLQASAANGMRASIFIQPKDGGPLQTYAIQFLEEAPKIAHLSLQVEKADSLKEDQTVKLSVRAHYQDGTQAVLPADKVTFSTSGEGEVAIRKGMLELHKPGAVTLNAEYEGAKDQVELTIQANTEKKIAQSIRPVNVVTDLHQEPSLPATVTVEYDKGFPKTHKVTWQAIPKEKLDSYQTFEVLGKVEGIDLEARAKVSVEGIVSVEEVSVTTPIAEAPQLPESVRTYDSNGHVSSAKVAWDAIRPEQYAKEGVFTVNGRLEGTQLTTKLHVRVSAQTEQGANISDQWTGSELPLAFASDSNPSDPVSNVNDKLISYNNQPANRWTNWNRTNPEASVGVLFGDSGILSKRSVDNLSVGFHEDHGVGVPKSYVIEYYVGKTVPTAPKNPSFVGNEDHVFNDSANWKPVTNLKAPAQLKAGEMNHFSFDKVETYAVRIRMVKADNKRGTSITEVQIFAKQVAAAKQGQTRIQVDGKDLANFNPDLTDYYLESVDGKVPAVTASVSNNGLATVVPSVREGEPVRVIAKAENGDILGEYRLHFTKDKSLLSHKPVAAVKQARLLQVGQALELPTKVPVYFTGKDGYETKDLTVEWEEVPAENLTKAGQFTVRGRVLGSNLVAEITVRVTDKLGETLSDNPNYDENSNQAFASATNDIDKNSHDRVDYLNDGDHSENRRWTNWSPTPSSNPEVSAGVIFRENGKIVERTVTQGKVQFFADSGTDAPSKLVLERYVGPEFEVPTYYSNYQAYDADHPFNNPENWEAVPYRADKDIAAGDEINVTFKAIKAKAMRWRMERKADKSGVAMIEMTFLAPSELPQESTQSKILVDGKELADFAENRQDYQITYKGQRPKVSVEENNQVASTVVDSGEDSFPVLVRLVSESGKQVKEYRIHLTKEKPVSEKTVAAVQEDLPKIEFVEKDLAYKTVEKKDSTLYLGETRVEQEGKVGKERIFTAINPDGSKEEKLREVVEVPTDRIVLVGTKPVAQEAKKPQVSEKADTKPIDSSEASQTNKAQLPSTGSAASQAAVAAGLTLLGLSAGLVVTKGKKED

SEQ ID NO: 46 - protein, 218 aa, Streptococcus pneumoniae
ALIFNTQIRNIFIVWNTNKYQVSQVSKEKLEENQDTEGNFDFDSVKAISSEAVLTSQWDAQKLPVIGGIAIPELEMNLPIFKGLDNVNLFYGAGTMKREQVMGEGNYSLASHHIFGVDNANKMLFSPLDNAKNGMKIYLTDKNKVYTYEIREVKRVTPDRVDEVDDRDGVNEITLVTCEDLAATERIIVKGDLKETKDYSQTSDEILTAFNQPYKQFY

SEQ ID NO: 47 - protein, 42 aa, Streptococcus pneumoniae
MLTDWQKVNGNWYYLNSNGAMVTGSQTIDGKVYNFASSGEWI

SEQ ID NO: 48 - protein, 680 aa, Streptococcus pneumoniae
ASDGTWQGKQYLKEDGSQAANEWVFDTHYQSWFYIKADANYAENEWLKQGDDYFYLKSGGYMAKSEWVEDKGAFYYLDQDGKMKRNAWVGTSYVGATGAKVIEDWVYDSQYDAWFYIKADGQHAEKEWLQIKGKDYYFKSGGYLLTSQWINQAYVNASGAKVQQGWLFDKQYQSWFYIKENGNYADKEWIFENGHYYYLKSGGYMAANEWIWDKESWFYLKFDGKIAEKEWVYDSHSQAWYYFKSGGYMAANEWIWDKESWFYLKFDGKMAEKEWVYDSHSQAWYYFKSGGYMTANEWIWDKESWFYLKSDGKIAEKEWVYDSHSQAWYYFKSGGYMTANEWIWDKESWFYLKSDGKMAEKEWVYDSHSQAWYYFKSGGYMAKNETVDGYQLGSDGKWLGGKATNKNAAYYQVVPVTANVYDSDGEKLSYISQGSVVWLDKDRKSDDKRLAITISGLSGYMKTEDLQALDASKDFIPYYESDGHRFYHYVAQNASIPVASHLSDMEVGKKYYSADGLHFDGFKLENPFLFKDLTEATNYSAEELDKVFSLLNINNSLLENKGATFKEAEEHYHINALYLLAHSALESNWGRSKIAKDKNNFFGITAYDTTPYLSAKTFDDVDKGILGATKWIKENYIDRGRTFLGNKASGMNVEYASDPYWGEKIASVMMKINEKLGGKD

SEQ ID NO: 49 - protein, 469 aa, Streptococcus pneumoniae
ANETEVAKTSQDTTTASSSSEQNQSSNKTQTSAEVQTNAAAYWDGDYYVKDDGSKAQSEWIFDNYYKAWFYINSDGRYSQNEWHGNYYLKSGGYMAQNEWIYDSNYKSWFYLKSDGAYAHQEWQLIGNKWYYFKKWGYMAKSQWQGSYFLNGQGAMIQNEWLYDPAYSAYFYLKSDGTYANQEWQKVGGKWYYFKKWGYMARNEWQGNYYLTGSGAMATDEVIMDGARYIFAASGELKEKKDLNVGWVHRDGKRYFFNNREEQVGTEHAKKIIDISEHNGRINDWKKVIDENKVDGVIVRLGYSGKEDKELAHNIKELNRLGIPYGVYLYTYAENETDAENDAKQTIELIKKYNMNLSYPIYYDVENWEYVNKSKRAPSDTDTWVKIINKYMDTMKQAGYQNVYVYSYRSLLQTRLKHPDILKHVNWVAAYTNALEWENPYYSGEKGWQYTSSEYMKGIQGRVDVSVWY

SEQ ID NO: 50 - protein, 471 aa, Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHLGLPISYTTSFLRDNVVATFQNSTDYVETKVTAYRNGDLLLDHSGAYVAQYYITWNELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 51 - protein, 471 aa, Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHLGLPISYTTSFLRDNVVATFQNSTDYVETKVTAYRNGDLLLDHSGAYVAQYYITWNELSYDHQGKEVLTPKAWDRNGQDLTAHFTTSIPLKGNVRNLSVKIRECTGLAFEWWRTVYEKTDLPLVRKRTISIWGTTLYPQVEDKVEND

SEQ ID NO: 52 - protein, 464 aa, Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHLGLPISYTTSFLRDNVVATFQNSTDYVETKVTAYRNGDLLLDHSGAYVAQYYITWNELSYDHQGKEVLTPKAWDRNGQDLTAHFTTSIPLKGNVRNLSVKIRECTGLAWEWWRTVYEKTDLPLVRKRTISIWGTTLYPQV

SEQ ID NO: 53 - protein, 366 aa, Streptococcus pneumoniae
AETTDDKIAAQDNKISNLTAQQQEAQKQVDQIQEQVSAIQAEQSNLQAENDRLQAESKKLEGEITELSKNIVSRNQSLEKQARSAQTNGAVTSYINTIVNSKSITEAISRVAAMSEIVSANNKMLEQQKADKKAISEKQVANNDAINTVIANQQKLADDAQALTTKQAELKAAELSLAAEKATAEGEKASLLEQKAAAEAEARAAAVAEAAYKEKRASQQQSVLASANTNLTAQVQAVSESAAAPVRAKVRPTYSTNASSYPIGECTWGVKTLAPWAGDYWGNGAQWATSAAAAGFRTGSTPQVGAIACWNDGGYGHVAVVTAVESTTRIQVSESNYAGNRTIGNHRGWFNPTTTSEGFVTYIYAD

SEQ ID NO: 54 - protein, 195 aa, Streptococcus pneumoniae
MKSITKKIKATLAGVAALFAVFAPSFVSAQESSTYTVKEGDTLSEIAETHNTTVEKLAENNHIDNIHLIYVDQELVIDGPVAPVATPAPATYAAPAAQDETVSAPVAETPVVSETVVSTVSGSEAEAKEWIAQKESGGSYTATNGRYIGRYQLTDSYLNGDYSAENQERVADAYVAGRYGSWTAAKNFWLNNGWY

SEQ ID NO: 55 - protein, 659 aa, Streptococcus pneumoniae
MKKNRVFATAGLVLLAAGVLAACSSSKSSDSSAPKAYGYVYTADPETLDYLISSKNSTTVVTSNGIDGLFTNDNYGNLAPAVAEDWEVSKDGLTYTYKIRKGVKWFTSDGEEYAEVTAKDFVNGLKHAADKKSEAMYLAENSVKGLADYLSGTSTDFSTVGVKAVDDYTLQYTLNQPEPFWNSKLTYSIFWPLNEEFETSKGSDFAKPTDPTSLLYNGPFLLKGLTAKSSVEFVKNEQYWDKENVHLDTINLAYYDGSDQESLERNFTSGAYSYARLYPTSSNYSKVAEEYKDNIYYTQSGSGIAGLGVNIDRQSYNYTSKTTDSEKVATKKALLNKDFRQALNFALDRSAYSAQINGKDGAALAVRNLFVKPDFVSAGEKTFGDLVAAQLPAYGDEWKGVNLADGQDGLFNADKAKAEFAKAKKALEADGVQFPIHLDVPVDQASKNYISRIQSFKQSVETVLGVENVVVDIQQMTSDEFLNITYYAANASSEDWDVSGGVSWGPDYQDPSTYLDILKTTSSETTKTYLGFDNPNSPSVVQVGLKEYDKLVDEAARETSDLNVRYEKYAAAQAWLTDSSLFIPAMASSGAAPVLSRIVPFTGASAQTGSKGSDVYFKYLKSQDKVVTKEEYEKAREKWLKEKAESNEKAQKELASHVK

SEQ ID NO: 56 - protein, 294 aa, Streptococcus pneumoniae
ACSKGSEGADLISMKGDVITEHQFYEQVKNNPSAQQVLLNMTIQKVFEKQYGSELDDKEVDDTIAEEKKQYGENYQRVLSQAGMTLETRKAQIRTSKLVELAVKKVAEAELTDEAYKKAFDEYTPDVTAQIIRLNNEDKAKEVLEKAKAEGADFAQLAKDNSTDEKTKENGGEITFDSASTEVPEQVKKAAFALDVDGVSDVITATGTQAYSSQYYIVKLTKKTEKSSNIDDYKEKLKTVILTQKQNDSTFVQSIIGKELQAANIKVKDQAFQNIFTQYIGGGDSSSSSSTSNE

SEQ ID NO: 57 - protein, 448 aa, Streptococcus pneumoniae
MKILPFIARGTSYYLKMSVKKLVPFLVVGLMLAAGDSVYAYSRGNGSIARGDDYPAYYKNGSQEIDQWRMYSRQCTSFVAFRLSNVNGFEIPAAYGNANEWGHRARREGYRVDNTPTIGSITWSTAGTYGHVAWVSNVMGDQIEIEEYNYGYTESYNKRVIKANTMTGFIHFKDLDGGSVGNSQSSTSTGGTHYFKTKSAIKTEPLASGTVIDYYYPGEKVHYDQILEKDGYKWLSYTAYNGSYRYVQLEAVNKNPLGNSVLSSTGGTHYFKTKSAIKTEPLVSATVIDYYYPGEKVHYDQILEKDGYKWLSYTAYNGSRRYIQLEGVTSSQNYQNQSGNISSYGSHSSSTVGWKKINGSWYHFKSNGSKSTGWLKDGSSWYYLKLSGEMQTGWLKENGLWYYLGSSGAMKTGWYQVSGKWYYSYSSGALAVNTTVDGYRVNSDGERV

SEQ ID NO: 58 - protein, 196 aa, Streptococcus pneumoniae
MIPVVIEQTSRGERSYDIYSRLLKDRIIMLTGPVEDNMANSVIAQLLFLDAQDSTKDIYLYVNTPGGSVSAGLAIVDTMNFIKADVQTIVMGMAASMGTVIASSGAKGKRFMLPNAEYMIHQPMGGTGGGTQQTDMAIAAEHLLKTRNTLEKILAENSGQSMEKVHADAERDNWMSAQETLEYGFIDEIMANNSLN

SEQ ID NO: 59 - protein, 318 aa, Streptococcus pneumoniae
MEINVSKLRTDLPQVGVQPYRQVHAHSTGNPHSTVQNEADYHWRKDPELGFFSHIVGNGCIMQVGPVDNGAWDVGGGWNAETYAAVELIESHSTKEEFMTDYRLYIELLRNLADEAGLPKTLDTGSLAGIKTHEYCTNNQPNNHSDHVDPYPYLAKWGISREQFKHDIENGLTIETGWQKNDTGYWYVHSDGSYPKDKFEKINGTWYYFDSSGYMLADRWRKHTDGNWYWFDNSGEMATGWKKIADKWYYFNEEGAMKTGWVKYKDTWYYLDAKEGAMVSNAFIQSADGTGWYYLKPDGTLADRPEFTVEPDGLITVK

SEQ ID NO: 60 - protein, 828 aa, Streptococcus pneumoniae
MQLEISNRKRVSMKINKKYLVGSAAALILSVCSYELGLYQARTVKENNRVSYIDGKQATQKTENLTPDEVSKREGINAEQIVIKITDQGYVTSHGDHYHYYNGKVPYDAIFSEELLMKDPNYKLKDEDIVNEVKGGYVIKVDGKYYVYLKDAAHADNVRTKEEINRQKQEHSQHREGGTPRNDGAVALARSQGRYTTDDGYIFNASDIIEDTGDAYIVPHGDHYHYIPKNELSASELAAAEAFLSGRGNLSNSRTYRRQNSDNTSRTNWVPSVSNPGTTNTNTSNNSNTNSQASQSNDIDSLLKQLYKLPLSQRHVESDGLVFDPAQITSRTARGVAVPHGDHYHFIPYSQMSELEERIARIIPLRYRSNHWVPDSRPEQPSPQPTPEPSPGPQPAPNLKIDSNSSLVSQLVRKVGEGYVFEEKGISRYVFAKDLPSETVKNLESKLSKQESVSHTLTAKKENVAPRDQEFYDKAYNLLTEAHKALFENKGRNSDFQALDKLLERLNDESTNKEKLVDDLLAFLAPITHPERLGKPNSQIEYTEDEVRIAQLADKYTTSDGYIFDEHDIISDEGDAYVTPHMGHSHWIGKDSLSDKEKVAAQAYTKEKGILPPSPDADVKANPTGDSAAAIYNRVKGEKRIPLVRLPYMVEHTVEVKNGNLIIPHKDHYHNIKFAWFDDHTYKAPNGYTLEDLFATIKYYVEHPDERPHSNDGWGNASEHVLGKKDHSEDPNKNFKADEEPVEETPAEPEVPQVETEKVEAQLKEAEVLLAKVTDSSLKANATETLAGLRNNLTLQIMDNNSIMAEAEKLLALLKGSNPSSVSKEKIN

SEQ ID NO: 61 - protein, 819 aa, Streptococcus pneumoniae
  misc_feature 578: ′Xaa′ is any amino acid, such as Lysine
MKINKKYLAGSVAVLALSVCSYELGRYQAGQDKKESNRVAYIDGDQAGQKAENLTPDEVSKREGINAEQIVIKITDQGYVTSHGDHYHYYNGKVPYDAIISEELLMKDPNYQLKDSDIVNEIKGGYVIKVNGKYYVYLKDAAHADNIRTKEEIKRQKQERSHNHNSRADNAVAAARAQGRYTTDDGYIFNASDIIEDTGDAYIVPHGDHYHYIPKNELSASELAAAEAYWNGKQGSRPSSSSSYNANPAQPRLSENHNLTVTPTYHQNQGENISSLLRELYAKPLSERHVESDGLIFDPAQITSRTARGVAVPHGNHYHFIPYEQMSELEKRIARIIPLRYRSNHWVPDSRPEEPSPQPTPEPSPSPQPAPSNPIDGKLVKEAVRKVGDGYVFEENGVSRYIPAKDLSAETAAGIDSKLAKQESLSHKLGTKKTDLPSSDREFYNKAYDLLARIHQDLLDNKGRQVDFEALDNLLERLKDVSSDKVKLVEDILAFLAPIRHPERLGKPNAQITYTDDEIQVAKLAGKYTAEDGYIFDPRDITSDEGDAYVTPHMTHSHWIKKDSLSEAERAAAQAYAXEKGLTPPSTDHQDSGNTEAKGAEAIYNRVKAAKKVPLDRMPYNLQYTVEVKNGSLIIPHYDHYHNIKFEWFDEGLYEAPKGYTLEDLLATVKYYVEHPNERPHSDNGFGNASDHVQRNKNGQADTNQTEKPSEEKPQTEKPEEETPREEKPQSEKPESPKPTEEPEESPEESEEPQVETEKVEEKLREAEDLLGKIQDPIIKSNAKETLTGLKNNLLFGTQDNNTIMAEAEKLLALLKESK

SEQ ID NO: 62 - protein, 853 aa, Streptococcus pneumoniae
MKINKKYLAGSVAVLALSVCSYELGRHQAGQVKKESNRVSYIDGDQAGQKAENLTPDEVSKREGINAEQIVIKITDQGYVTSHGDHYHYYNGKVPYDAIISEELLMKDPNYQLKDSDIVNEIKGGYVIKVDGKYYVYLKDAAHADNIRTKEEIKRQKQERSHNHNSRADNAVAAARAQGRYTTDDGYIFNASDIIEDTGDAYIVPHGDHYHYIPKSDLSASELAAAQAYWNGKQGSRPSSSSSHNANPAQPRLSENHNLTVTPTYHQNQGENISSLLRELYAKPLSERHVESDGLIFDPAQITSRTANGVAVPHGDHYHFIPYSQLSPLEEKLARIIPLRYRSNHWVPDSRPEQPSPQSTPEPSPSPQPAPNPQPAPSNPIDEKLVKEAVRKVGDGYVFEENGVPRYIPAKDLSAETAAGIDSKLAKQESLSHKLGAKKTDLPSSDREFYNKAYDLLARIHQDLLDNKGRQVDFEALDNLLERLKDVSSDKVKLVDDILAFLAPIRHPERLGKPNAQITYTDDEIQVAKLAGKYTTEDGYIFDPRDITSDEGDAYVTPHMTHSHWIKKDSLSEAERAAAQAYAKEKGLTPPSTDHQDSGNTEAKGAEAIYNRVKAAKKVPLDRMPYNLQYTVEVKNGSLIIPHYDHYHNIKFEWFDEGLYEAPKGYSLEDLLATVKYYVEHPNERPHSDNGFGNASDHVQRNKNGQADTNQTEKPNEEKPQTEKPEEDKEHDEVSEPTHPESDEKENHVGLNPSADNLYKPSTDTEETEEEAEDTTDEAEIPQVEHSVINAKIAEAEALLEKVTDSSIRQNAVETLTGLKSSLLLGTKDNNTISAEVDSLLALLKESQPTPIQ

SEQ ID NO: 63 - protein, 1039 aa, Streptococcus pneumoniae
MKFSKKYIAAGSAVIVSLSLCAYALNQHRSQENKDNNRVSYVDGSQSSQKSENLTPDQVSQKEGIQAEQIVIKITDQGYVTSHGDHYHYYNGKVPYDALFSEELLMKDPNYQLKDADIVNEVKGGYIIKVDGKYYVYLKDAAHADNVRTKDEINRQKQEHVKDNEKVNSNVAVARSQGRYTTNDGYVFNPADIIEDTGNAYIVPHGGHYHYIPKSDLSASELAAAKAHLAGKNMQPSQLSYSSTASDNNTQSVAKGSTSKPANKSENLQSLLKELYDSPSAQRYSESDGLVFDPAKIISRTPNGVAIPHGDHYHFIPYSKLSALEEKIARRVPISGTGSTVSTNAKPNEVVSSLGSLSSNPSSLTTSKELSSASDGYIFNPKDIVEETATAYIVRHGDHFHYIPKSNQIGQPTLPNNSLATPSPSLPINPGISHEKHEEDGYGFDANRIIAEDESGFIMSHGNHNHYFFKKDLTEEQIKAAQKHLEEVKTSHNGLDSLSSHEQDYPGNAKEMKDLDKKIEEKIAGIMKQYGVKRESIVVNKEKNAIIYPHGDHHHADPIDEHKPVGIGHSHSNYELFKPEEGVAKKEGNKVYTGEELTNVVNLLKNSTFNNQNFTLANGQKRVSFSFPPELEKKLGINMLVKLITPDGKVLEKVSGKVFGEGVGNIANFELDQPYLPGQTFKYTIASKDYPEVSYDGTFTVPTSLAYKMASQTIFYPFHAGDTYLRVNPQFAVPKGTDALVRVFDEFHGNAYLENNYKVGEIKLPIPKLNQGTTRTAGNKIPVTFMANAYLDNQSTYIVEVPILEKENQTDKPSILPQFKRNKAQENSKLDEKVEEPKTSEKVEKEKLSETGNSTSNSTLEEVPTVDPVQEKVAKFAESYGMKLENVLFNMDGTIELYLPSGEVIKKNMADFTGEAPQGNGENKPSENGKVSTGTVENQPTENKPADSLPEAPNEKPVKPENSTDNGMLNPEGNVGSDPMLDPALEEAPAVDPVQEKLEKFTASYGLGLDSVIFNMDGTIELRLPSGEVIKKNLSDLIA

SEQ ID NO: 64 - protein, 1876 aa, Streptococcus pneumoniae
MFKKDRFSIRKIKGVVGSVFLGSLLMAPSVVDAATYHYVNKEIISQEAKDLIQTGKPDRNEVVYGLVYQKDQLPQTGTEASVLTAFGLLTVGSLLLIYKRKKIASVFLVGTMGLVVLPSAGAVDPVATLALASREGVVEMEGYRYVGYLSGDILKTLGLDTVLEETSAKPGEVTVVEVETPQSTTNQEQARTENQVVETEEAPKEEAPKTEESPKEEPKSEVKPTDDTLPKVEEGKEDSAEPAPVEEVGGEVESKPEEKVAVKPESQPSDKPAEESKVEQAGEPVAPRKDEQAPVEPENQPEAPEEEKAVEETPKQEESTPDTKAEETVEPKEETKTAKGTQEEGKEGQAPVQEVNPEYKVTTGTVEKSTESELDFTTEVVPDDTKYVDEEVVERQGSKGVQVTKTTYETVEVVETDKVLSTTTEVKTPVVPKVVKKGTKPVETREEVIPFATKEQEDDTLKRGTRQVAQEGVNGKKQITETYKTIRGEKTNEAPTVEETVLQAPQDEIIKKGTKGLEKPTLQWANTEKDVLKKSATASYTLTKPAGVEIKSIKLALKDKDGQLVKEVTVAENNLNATLDKLKYYQGYTLSTTMVYDRGEGEETEKLEDKQIQLDLKKVEIKNIKETSLMNVDAEGNETDKSLLSEKPTDVSQLYLRVTTHDNKVTRLAVSSVEEVVVDGKTLYKVVAKAPDLVQRRADDTLSEEYVHYFEKQLPKVNNVYYNFNELVKDMQANPMGEFKLGADLNAVNVKPAGKAYVMAKFRGTLSSVENHQYTIHNLERPLFNEAEGATLKNFNLGNVNINMPWADKVAPIGNMFKKSTLENIKVVGSVTGNNDVTGAVNKLDEANMRNVAFIGKINSLGDKGWWSGGLVSESWRSNTDSVYFDGDIVGNNSKFGGLVAKVNHGSNQWDVKQKGRLTNSVVKGTMTLKNHGQSGGLVHENYDWGWVENNISMMKVNNGEIMYGSGSIDGDPYFGFDYFKNNYYVKDVATGESTYKRSKQIQSISQAEADAKIANMGITANTFAIQDPVVNKLNRIIDRDSEYKAIQDYQETRNLAYRNLEKLQPFYNKEWIVNQGNKLTDESNLVKKTVLSVTGMKSGQFVTDLSSVDKIMIHYADGTKEEFGVSAISDSRVKQVKEYNVDDLGVVYTPNMVDKNRDSLITKVKEKLSSVALDSAEVKSITNNPASLYLEESFAEVRETLDKLVKSLLENEDHQLNSDEVAEKALLKKVEDNKAKIILALTYLNRYYGIDYDGLNFKHLMMFKPDFYGKTPSILDFLIRIGSAEKNLKGDRSLEAYREVIGGTIGKGELNGLLGYNMRLFTKYTDLNDWFIHAAKNVYVSEPETTTEDFKDKRHRIYDGLNNDVHGRMILPLLNLKKAHIFVISTYNTIAFSSFEKYGKNTEEERNAYKAEIDRVAKAQQRYLDFWSRLALPKVRNQLLKSQNSVPTPVWDNQVYVGLGGANRMGYGDGGRVVTPVRELFGPTDRWHQINWNMGAMAKIYERPWKDDQVYFMVTNMMEPFGISAFTHETTHVNDRMAYYGGDWHREGTDLEAFAQGMLQTPDKSTTNGEYGALGINMAYERKNDGEQLYNYDPEKLDSREKIDSYMKNYNESMMMLDYLEASAVIRQNLSDNSKWFKKMDKEWRTNADRNRLIGEPHQWDKLRDLTEEEKKLPIDSIDKLVENNFVTLHGMPKNGRYRTEGFDSSYQPVNMMAGVFGGNTSKSTVGSISFKHNAFRMWGYYGYENGFIPYVSNKLKGAANKENKGLLGDDFIIKKVSKNQFQNLEEWKKHWYHEVYDKAQKGFVEIEVDGVKISTYAQLQSLFEEAVSKDLAGMDDKNIKNHYQYTENLKWKIYKQLLKNTDGFSSDLFTAPQA

SEQ ID NO: 65 - protein, 448 aa, Streptococcus pneumoniae
MKILPFIARGTSYYLKMSVKKLVPFLVVGLMLAAGDSVYAYSRGNGSIARGDDYPAYYKNGSQEIDQWRMYSRQCTSFVAFRLSNVNGFEIPAAYGNANEWGHRARREGYRVDNTPTIGSITWSTAGTYGHVAWVSNVMGDQIEIEEYNYGYTESYNKRVIKANTMTGFIHFKDLDSGSVGNSQSSASTGGTHYFKTKSAIKTEPLVSATVIDYYYPGEKVHYDQILEKDGYKWLSYTAYNGSYRYVQLEAVNKNPLGNSVLSSTGGTHYFKIKSAIKTEPLVSATVIDYYYPGEKVHYDQILEKDGYKWLSYTAYNGSRRYIQLEGVTSSQNYQNQSGNISSYGSNNSSTVGWKKINGSWYHFKSNGSKSTGWLKDGSSWYYLKLSGEMQTGWLKENGSWYYLGSSGAMKTGWYQVSGEWYYSYSSGALAINTTVDGYRVNSDGERV

SEQ ID NO: 66 - protein, 544 aa, Streptococcus pneumoniae
MAETTDDKIAAQDNKISNLTAQQQEAQKQVDQIQEQVSAIQAEQSNLQAENDRLQAESKKLEGEITELSKNIVSRNQSLEKQARSAQTNGAVTSYINTIVNSKSITEAISRVAAMSEIVSANNKMLEQQKADKKAISEKQVANNDAINTVIANQQKLADDAQALTTKQAELKAAELSLAAEKATAEGEKASLLEQKAAAEAEARAAAVAEAAYKEKRASQQQSVLASANTNLTAQVQAVSESAAAPVRAKVRPTYSTNASSYPIGECTWGVKTLAPWAGDYWGNGAQWATSAAAAGFRTGSTPQVGAIACWNDGGYGHVAVVTAVESTTRIQVSESNYAGNRTIGNHRGWFNPTTTSEGFVTYIYADGSGSGGGGVSAQESSTYTVKEGDTLSEIAETHNTTVEKLAENNHIDNIHLIYVDQELVIDGPVAPVATPAPATYAAPAAQDETVSAPVAETPVVSETVVSTVSGSEAEAKEWIAQKESGGSYTATNGRYIGRYQLTDSYLNGDYSAENQERVADAYVAGRYGSWTAAKNFWLNNGWY

SEQ ID NO: 67 - protein, 554 aa, Streptococcus agalactiae
MKLSKKLLFSAAVLTMVAGSTVEPVAQFATGMSIVRAAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPSIPNTGGIGTAIFVAIGAAVMAFAVKGMKRRTKDN

SEQ ID NO: 68 - protein, 251 aa, Streptococcus pneumoniae
ETTDDKIAAQDNKISNLTAQQQEAQKQVDQIQEQVSAIQAEQSNLQAENDRLQAESKKLEGEITELSKNIVSRNQSLEKQARSAQTNGAVTSYINTIVNSKSITEAISRVAAMSEIVSANNKMLEQQKADKKAISEKQVANNDAINTVIANQQKLADDAQALTTKQAELKAAELSLAAEKATAEGEKASLLEQKAAAEAEARAAAVAEAAYKEKRASQQQSVLASANTNLTAQVQAVSESAAAPVRAKVRP

SEQ ID NO: 69 - protein, 315 aa, Streptococcus pneumoniae
YLILLASLVLVAASLIWILSRTPATIAIPDVAGQTVAEAKATLKKANFEIGEEKTEASEKVEEGRIIRTDPGAGTGRKEGTKINLVVSSGKQSFQISNYVGRKSSDVIAELKEKKVPDNLIKIEEEESNESEAGTVLKQSLPEGTTYDLSKATQIVLTVAKKATTIQLGNYIGRNSTEVISELKQKKVPENLIKIEEEESSESEPGTIMKQSPGAGTTYDVSKPTQIVLTVAKKVTSVAMPSYIGSSLEFTKNNLIQIVGIKEANIEVVEVTTAPAGSVEGMVVEQSPRAGEKVDLNKTRVKISIYKPKTTSATP

SEQ ID NO: 70 - protein, 289 aa, Streptococcus pneumoniae
ASGKKDTTSGQKLKVVATNSIIADITKNIAGDKIDLHSIVPIGQDPHEYEPLPEDVKKTSEADLIFYNGINLETGGNAWFTKLVENAKKTENKDYFAVSDGVDVIYLEGQNEKGKEDPHAWLNLENGIIFAKNIAKQLSAKDPNNKEFYEKNLKEYTDKLDKLDKESKDKFNKIPAEKKLIVTSEGAFKYFSKAYGVPSAYIWEINTEEEGTPEQIKTLVEKLRQTKVPSLFVESSVDDRPMKTVSQDTNIPIYAQIFTDSIAEQGKEGDSYYSMMKYNLDKIAEGLAK

SEQ ID NO: 71 - DNA, 26 bp, Artificial Sequence
  Immunostimulatory oligonucleotide
ncncncncncncncncncncncncnc

SEQ ID NO: 72 - protein, 11 aa, Artificial Sequence
  Cationic oligopeptide
KLKLLLLLKLK